(19) United States
(12) Patent Application Publication
(10) Pub. No.: US 2026/0000317 A1
(43) Pub. Date: Jan. 1, 2026

(54) DEVICES, SYSTEMS, AND METHODS FOR PRESSURE MAPPING A FOOT OF A USER

(71) Applicant: Liquid Wire Inc., Portland, OR (US)

(72) Inventors: Mark William Ronay, Portland, OR (US); Trevor Antonio Rivera, Portland, OR (US); Jorge E. Carbo, Jr., Portland, OR (US); Michael Adventure Hopkins, Portland, OR (US); Edward Martin Godshalk, Newberg, OR (US); Charles J. Kinzel, Portland, OR (US); Michael Jasper Wallans, Portland, OR (US); Katherine M. Nelson, Portland, OR (US); Jesse Michael Martinez, Milwaukie, OR (US)

(73) Assignee: Liquid Wire Inc., Portland, OR (US)

(21) Appl. No.: 19/045,390

(22) Filed: Feb. 4, 2025

Related U.S. Application Data
(63) Continuation of application No. PCT/US23/71790, filed on Aug. 7, 2023.
(60) Provisional application No. 63/370,603, filed on Aug. 5, 2022.

(51) Int. Cl. A61B 5/103; A61B 5/00

(52) U.S. Cl. CPC A61B 5/1038; A61B 5/6829; A61B 5/746; A61B 2562/0261; A61B 2562/168; A61B 2562/227

(57) ABSTRACT

A system configured to control a virtual representation of a user within a virtual environment is disclosed herein. The system can include a foot base configured to receive a user input. The foot base can include a flexible circuit including an encapsulated deformable conductor. The system can further include a processor communicably coupled to the flexible circuit, and a memory configured to store instructions that, when executed by the processor, cause the processor to: receive a signal from the flexible circuit, determine an electrical parameter based on the received signal, correlate the determined electrical parameter to a physical parameter of the flexible circuit, and alter the virtual representation of the user based on the correlation.

### CROSS-REFERENCE TO RELATED APPLICATIONS

The present application is a continuation of and claims the benefit of priority from PCT/US2023/071790, filed on Aug. 7, 2023, which claims the benefit of priority from U.S. Provisional Patent Application No. 63/370,603 filed Aug. 5, 2022, and entitled “DEVICES, SYSTEMS, AND METHODS FOR PRESSURE MAPPING A FOOT OF A USER,”. The disclosures of both applications are herein incorporated by reference in their entireties.

All applications referenced herein are relevant to the subject matter disclosed herein and are hereby incorporated by reference in their entirety, regardless of the specific portion of the specification in which they are referenced.

### FIELD

The present disclosure is generally related to flexible circuits and, more particularly, is directed to flexible circuits that can be either integrated into wearable articles or mats for the purposes of navigating a virtual environment based on the physical motions of a user in a real environment.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, a system configured to control a virtual representation of a user within a virtual environment is disclosed herein. The system can include a foot base configured to receive a user input. The foot base can include a flexible circuit including an encapsulated deformable conductor. The system can further include a processor communicably coupled to the flexible circuit, and a memory configured to store instructions that, when executed by the processor, cause the processor to: receive a signal from the flexible circuit, determine an electrical parameter based on the received signal, correlate the determined electrical parameter to a physical parameter of the flexible circuit, and alter the virtual representation of the user based on the correlation.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a perspective view of a fluid-fillable circuit, in accordance with one non-limiting aspect of the present disclosure;

FIG. 2 illustrates a cross-sectioned view of the fluid-fillable circuit of FIG. 1, in accordance with at least one non-limiting aspect of the present disclosure;

FIGS. 3A-3D collectively illustrate an assembly of the fluid-fillable circuit of FIG. 1, in accordance with at least one non-limiting aspect of the present disclosure;

FIG. 4 illustrates a perspective view of another inflatable circuit, in accordance with at least one non-limiting aspect of the present disclosure;

FIGS. 5A-5D collectively illustrate an assembly of the fluid-fillable circuit of FIG. 4, in accordance with at least one non-limiting aspect of the present disclosure;

FIG. 6 illustrates a perspective view of another inflatable circuit, in accordance with at least one non-limiting aspect of the present disclosure;

FIGS. 7A-7D collectively illustrate an assembly of the fluid-fillable circuit of FIG. 6, in accordance with at least one non-limiting aspect of the present disclosure;

FIG. 8 illustrates a perspective view of another inflatable circuit, in accordance with at least one non-limiting aspect of the present disclosure;

FIGS. 9A-9D collectively illustrate an assembly of the fluid-fillable circuit of FIG. 8, in accordance with at least one non-limiting aspect of the present disclosure;

FIG. 10 illustrates a partially fluid-fillable circuit, in accordance with at least one non-limiting aspect of the present disclosure;

FIG. 11 illustrates an assembly of the partially fluid-fillable circuit of FIG. 10, in accordance with at least one non-limiting aspect of the present disclosure;

FIG. 12 illustrates another partially fluid-fillable circuit, in accordance with at least one non-limiting aspect of the present disclosure;

FIG. 13 illustrates an assembly of the partially fluid-fillable circuit of FIG. 12, in accordance with at least one non-limiting aspect of the present disclosure;

FIG. 14 illustrates another assembly of another partially fluid-fillable circuit, in accordance with at least one non-limiting aspect of the present disclosure.

FIG. 15 illustrates another partially fluid-fillable circuit, in accordance with at least one non-limiting aspect of the present disclosure;

FIG. 16 illustrates a cross-sectioned side view of the partially fluid-fillable circuit of FIG. 15, in accordance with at least one non-limiting aspect of the present disclosure;

FIG. 17 illustrates an assembly of the partially fluid-fillable circuit of FIG. 15, in accordance with at least one non-limiting aspect of the present disclosure;

FIG. 18 illustrates an example foot base configured for the simulation of physical motions in a virtual environment, according to at least one non-limiting aspect of the present disclosure;

FIGS. 19A-19B illustrate example trace patterns of electrical features of the foot base of FIG. 18, In accordance with at least one non-limiting aspect of the present disclosure;

FIG. 20 illustrates an example embodiment of the foot base of FIG. 18, according to at least one non-limiting aspect of the present disclosure;

FIG. 21 illustrates another example embodiment of the foot base of FIG. 18, according to at least one non-limiting aspect of the present disclosure;

FIG. 22 illustrates another example foot base configured for the simulation of physical motions in a virtual environment, according to at least one non-limiting aspect of the present disclosure;

FIG. 23 illustrates another example foot base configured for the simulation of physical motions in a virtual environment, according to at least one non-limiting aspect of the present disclosure;

FIGS. 24A and 24B illustrate a flexible circuit configured for use with a wearable article, in accordance with at least one non-limiting aspect of the present disclosure;

FIG. 25 illustrates a partially-assembled wearable article featuring the flexible circuit of FIG. 24, in accordance with at least one non-limiting aspect of the present disclosure;

FIG. 26 illustrates the wearable article of FIG. 25, fully-assembled, in accordance with at least one non-limiting aspect of the present disclosure;

FIG. 27 illustrates another example foot base configured for the simulation of physical motions in a virtual environment, according to at least one non-limiting aspect of the present disclosure;

FIG. 28 is an enlarged view of the portion A of the example foot base of FIG. 27, according to at least one non-limiting aspect of the present disclosure;

FIG. 29 is a block diagram of an example foot base incorporated into a system, according to at least one non-limiting aspect of the present disclosure;

FIGS. 30A and 30B illustrate one implementation of another partially fluid-fillable circuit, in accordance with at least one non-limiting aspect of the present disclosure;

FIG. 31 illustrates an assembly of the partially fluid-fillable circuit of FIGS. 30A and 30B, in accordance with at least one non-limiting aspect of the present disclosure; and

FIGS. 32A and 32B, a such capacitive touch interface configured for use with a partially-fluid-fillable circuit is depicted in accordance with at least one non-limiting aspect of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms. In the following description, like reference characters designate like or corresponding parts throughout the several views of the drawings.

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves any and all copyrights disclosed herein.

Electronic circuits that are flexible and deformable have emerged as a means of innovating conventional electronics and introducing electronics into new products and applications. However, it would be beneficial for flexible electronic circuits to form a sealed, internal cavity, which can be filled with a compressible fluid. Such circuits could expand and contract in accordance with the selective insertion and/or removal of the fluid from the internal cavity. Moreover, the change in circuit geometry could lead to a subsequent change in electrical parameters generated across the inflatable circuit, which could be used to characterize a structural parameter or condition of the circuit, as desired. Indeed, inflatable circuits could provide numerous benefits for airbags, bladders, and/or cushions, which could be calibrated monitored, and even controlled based on measured electrical parameters.

While certain electronic components typically have some inherent flexibility, that flexibility is typically constrained both in the amount the components can flex, their resilience in flexing, and the number of times the electronic components can flex before the electronic components deteriorate or break. Consequently, the utility of such electronic components in various environments may be limited, either by reliability or longevity or by the ability to function at all. Moreover, the lateral size of such components may result in additional stresses placed on the component.

The use of conductive gel, however, provides for electronic components that are flexible and deformable while maintaining resiliency. Some such devices, systems, and methods are disclosed in U.S. Provisional Patent Application No. 63/272,487, titled DEVICES, SYSTEMS, AND METHODS FOR MAKING AND USING A FLUID-FILLABLE CIRCUIT, filed Oct. 27, 2021, the disclosure of which is hereby incorporated by reference in its entirety.

Moreover, the operational flexing, stretching, deforming, or other physical manipulation of a conductive trace formed from conductive gel may produce predictable, measurable changes in the electrical characteristics of the trace. By measuring the change in resistance or impedance of such a trace the change in length of the trace may be inferred. By combining the changes in lengths of multiple traces, the relative movement of points on a two-dimensional surface may be calculated.

A two-dimensional strain sensor has been developed that utilizes a network of conductive gel traces, the individual electrical characteristics of which translates to a relative length or other orientation of the trace. By combining the electrical characteristics, e.g., by triangulating or other mathematical process, the relative location of various points on a two-dimensional surface may be determined. By measuring such electrical characteristics repeatedly over time, the motion of the points may be determined, providing for the capacity for real-time motion capture of the points on the strain sensor. By scaling the network of traces and/or increasing the number of strain sensor and placing the strain sensors on an object, motion capture the object may be obtained in real-time.

Aspects of the present disclosure may provide a series of strain/pressure sensors and/or flexible circuits (e.g., inflatable circuits) in or on a wearable article (e.g., shoe or sock) or a mat to enable a user to navigate a virtual environment based on physical motions of the user in a real environment. For example, leaning the foot of the user in any direction can be picked up by the sensors/circuits and translated to a locomotive motion in the virtual environment. This can allow the user limitless movement in the virtual environment while the user is physically confined to a limited space. In some non-limiting aspects, using a series of sensors/circuits, a pressure mapping of the bottom of the foot can be created. This mapping can be used to interpret changes in pressure to motion in any direction, for example, on an x-y plane. In this way, aspects of the present disclosure may provide a more natural, comfortable, and instinctual solution for locomotion in a virtual environment.

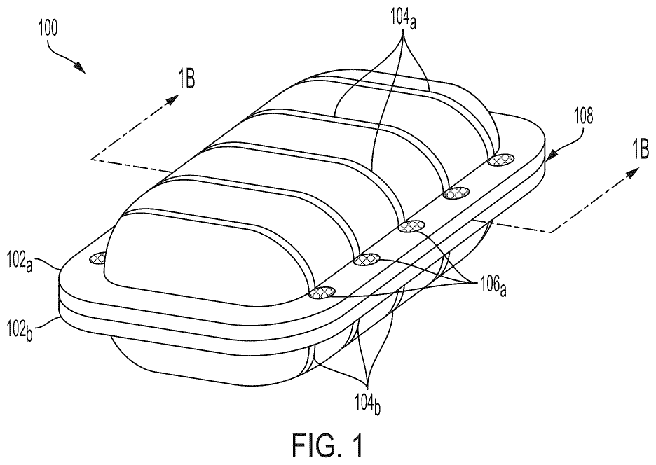

Examples of some fluid-fillable circuits according to the present disclosure are illustrated in FIGS. 1-9D. According to a non-limiting aspect of FIG. 1, a fluid-fillable circuit 100 can include a first layup 102ₐ, and a second layup 102_{b}. The first layup 102ₐ can include a first plurality of traces 104ₐ and the second layup 102_{b} can include a second plurality of traces 104. The first layup 102ₐ of FIG. 1 can be placed on top of—and mechanically coupled to—the second layup 102_{b} such that selected features—for example, the first plurality of traces 104ₐ and the second plurality of traces 104_{b}, are aligned. For example, each trace 104ₐ of the first plurality of traces 104ₐ can be electrically coupled to a corresponding trace 104_{b} of the second plurality of traces 104_{b} via a plurality of electrical vias 106ₐ and 106_{b} (not shown) positioned at a mechanical interface of the first layup 102ₐ and the second layup 102_{b}. However, in other non-limiting aspects, the first layup 102ₐ can be mechanically coupled, fused, or otherwise integrated to the second layup 102_{b} and a single plurality of traces can be deposited and/or coupled to both the first layup 102ₐ and the second layup 102_{b}, such that each trace of the plurality traverses the entire perimeter collectively defined by the first layup 102ₐ and the second layup 102_{b}.

According to the non-limiting aspect of FIG. 1, the vias 106 of the first layup 102ₐ can be dimensioned and positioned such that they substantially correspond to and align with the vias (not shown) of the second layup 102_{b}. However, in other non-limiting aspects, vias 106 of the first layup 102ₐ can be alternately configured relative to the vias 106 of the second layup 102_{b}. For example, the vias 106 of the first layup 102ₐ can be larger than the vias 106 of the second layup 102_{b} to account for tolerances and/or misalignment. Alternatively and/or additionally, the vias 106 of the first layup 102ₐ may be intentionally misaligned relative to the vias 106 of the second layup 102_{b}.

Still referring to FIG. 1, the first layup 102ₐ and the second layup 102_{b} of FIG. 1 can be configured such that either the first layup 102ₐ or the second layup 102_{b} can be positioned on top of the other. However, in other non-limiting aspects, either the first layup 102ₐ or the second layup 102ₐ can be specifically configured as a top portion or a bottom portion of the fluid-fillable circuit 100. For example, it may be preferable for a top portion or a bottom portion of the fluid-fillable circuit 100 to have specifically configured dimensions, mechanical features, and/or electrical features. As such, either the first layup 102ₐ or the second layup 102_{b} can be configured to include such features, rendering them exclusively suitable for placement on the top portion or a bottom portion of the fluid-fillable circuit 100. Some non-limiting examples of “other features” that can be added to the first layup 102ₐ or the second layup 102_{b} include electronic components, portions of the trace pattern, trace breaks with vias at trace endpoints, and/or a predetermined dimension of the first layup 102ₐ or the second layup 102_{b} when the fluid-fillable circuit 100 is, e.g., either inflated or deflated, amongst others. Alternatively and/or additionally, the first layup 102ₐ of FIG. 1 can be arranged relative to the second layup 102_{b} such that other features of the first layup 102ₐ are preferably aligned with other features of the second layup 102_{b}.

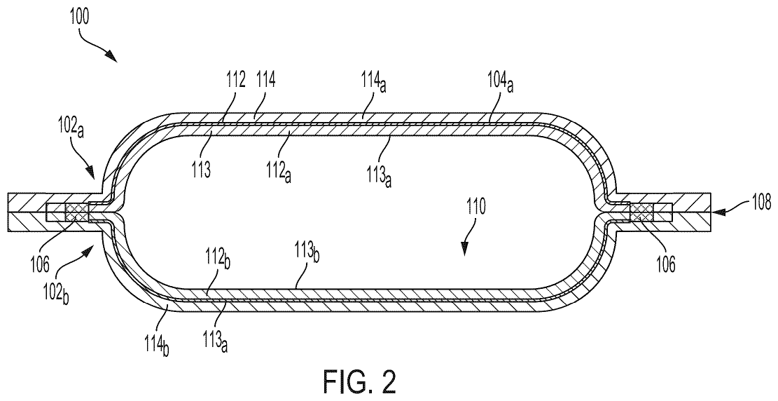

As used herein, the term “inflate” shall include the introduction of any foreign substance into an internal cavity 110 (FIG. 2) defined by the first and second layups 102ₐ, 102_{b}. For example, as used herein, “inflation” can include the introduction of a compressible fluid, a non-compressible fluid, a foam, and/or particles, amongst other media, into an internal cavity 110 (FIG. 2) of the circuit 100. Likewise, the term “deflate” shall include the removal of any foreign substance from the internal cavity 110 (FIG. 2) defined by the first and second layups 102ₐ, 102_{b}.

According to the non-limiting aspect of FIG. 1, the first layup 102ₐ and the second layup 102_{b} can be composed of flexible and stretchable materials, such as those disclosed by U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. Specifically, the first layup 102ₐ and the second layup 102_{b} can be fabricated from a flexible or stretchable material such as a natural rubber, a synthetic rubber, a flexible plastic, a silicone based material (e.g., polydimethylsiloxane (“PDMS”), thermoplastic polyurethane (“TPU”), ethylene propylene diene terpolymer (“EPDM”), neoprene, polyethylene terephthalate (“PET”), etc.), a flexible composite material, and/or a naturally flexible materials, such as a leather, for example. For example, the first layup 102ₐ and/or the second layup 102_{b} can be fabricated from a resilient, albeit stretchable TPU, such as Lubrizol® Estane® 58000 series (e.g., 58238), amongst others. Alternatively, the first layup 102ₐ and/or the second layup 102_{b} can be formed from a flexible, though comparatively more rigid material, such as Lubrizol® Estane® S375D, amongst others.

Since each trace 104ₐ of the first plurality of traces 104ₐ can be electrically coupled to a corresponding trace 104_{b} of the second plurality of traces 104_{b} via the plurality of electrical vias 106ₐ and 106_{b}, according to some non-limiting aspects, it may be preferable to fill the vias 106ₐ, 106_{b} with a deformable conductor configured to convey electrical energy between corresponding traces 104ₐ, 104_{b}. Such a deformable conductor is represented via the cross-hatching in FIGS. 1-3D. The deformable conductor placed in the vias 106ₐ, 106_{b} can be the same as, or similar to, the deformable conductors used for the traces 104ₐ, 104_{b}. Alternately, the deformable conductor placed in the vias 106ₐ, 106_{b} can be different than the deformable conductors used for the traces 104ₐ, 104_{b}. However, the use of cross-hatching is merely illustrative and the particular nature and/or absence of cross-hatching in any of the figures shall not be construed as limiting to the deformable conductor and/or the vias 106ₐ, 106_{b}, themselves. According to other non-limiting aspects, it shall be appreciated that the vias 106ₐ, 106_{b} can be alternately configured to convey electrical energy between corresponding traces 104ₐ, 104_{b} of the circuit 100.

As used herein, the term “deformable conductor” shall include but shall not be limited to, any conductor that is in a fluid state, or any conductor that can transition into a fluid state when an external stimulus (e.g. a strain, etc.) is applied to either the conductor or a structure surrounding the conductor, wherein the conductor is also capable of returning to a pre-stimulus state. Alternatively and/or additionally, a “deformable conductor” can include an elastic property greater than an elastic property of its surrounding structure, such that the conductor will not plastically deform before the surrounding structure plastically deforms. In other words, a “deformable conductor,” as used herein, shall include a rigidity that does not preclude the fluid-fillable circuits disclosed herein from undergoing the requisite transition between a first (e.g., deflated) state to a second (e.g., inflated) state while preserving electrical communication throughout the circuit in either the first or second state. In this regard, a “deformable conductor” is distinguished from conventional conductors, such as copper wires, which would prevent the transition between the first and second states, or whose rigidity might break the electrical communication throughout the circuit during said transitions.

In some aspects, the properties of the deformable conductive material and/or the properties of the layers surrounding the patterns of the deformable conductive material may be adjusted and/or optimized to ensure that the patterns of deformable conductive material heal upon unitization of the surrounding layers. For example, the deformable conductive material may be optimized to have a viscosity such that the deformable conductive material is able to heal upon unitization of the layers but not such that the deformable conductive material overly deforms and does not achieve the intended pattern. As another example, an adhesive characteristics and/or viscosity of the deformable conductive material may be optimized such that it remains on the substrate layer upon removal of a removable stencil and but does not adhere to the channels of the stencil thereby lifting the deformable conductive material off of the substrate layer. In some aspects, a viscosity of the deformable conductive material may, when under high shear (e.g., in motion), be in a range of about 10 Pascal seconds (Pa*s) and 500 Pa*s, such as a range of 50 Pa*s and 300 Pa*s, and/or may be about 50 Pa*s, about 60 Pa*s, about 70 Pa*s, about 80 Pa*s, about 90 Pa*s, about 100 Pa*s, about 110 Pa*s, about 120 Pa*s, about 130 Pa*s, about 140 Pa*s, about 150 Pa*s, about 160 Pa*s, about 170 Pa*s, about 180 Pa*s, about 190 Pa*s, or about 200 Pa*s. In some aspects, a viscosity of the deformable conductive material may, when under low shear (e.g., at rest), be in a range of 1,000,000 Pa*s and 40,000,000 Pa*s and/or may be about 10,000,000 Pa*s, about 20,000,000 Pa*s, about 30,000,000 Pa*s, or about 40,000,000 Pa*s.

Similarly, the traces 104ₐ, 104_{b} of the circuit 100 of FIG. 1 can include a deformable, conductive material, such as those disclosed in International Patent Application No. PCT/US2017/019762 titled LIQUID WIRE, which was filed on Feb. 27, 2017 and published on Sep. 8, 2017 as International Patent Publication No. WO2017/151523A1, the disclosure of which is hereby incorporated by reference in its entirety. For example, each trace 104ₐ, 104_{b} can include a variety of forms, such as a liquid, a paste, a gel, and/or a powder, amongst others that would enable the traces 104ₐ, 104_{b} to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium indium alloy). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring traces 104ₐ, 104_{b} of varying forms and/or compositions to achieve the benefits disclosed herein.

In further reference to FIG. 1, the first layup 102ₐ can be configured to mechanically interface the second layup 102_{b} such that a seal 108 can be formed between the first layup 102ₐ and the second layup 102_{b}. The seal 108 can be formed via a process configured to attach an outer perimeter of the first layup 102ₐ to an outer perimeter of the second layup 102_{b}, including any known process that uses heat, pressure, radio-frequency energy, and/or additional materials for attachment (e.g., welding, soldering, fusing, stitching, adhesives, etc.). According to some non-limiting aspects, the seal 108 can be formed using a radio-frequency weld procedure, an adhesive (e.g., a thermoset, a thermoplastic adhesive film, etc.), or a thermos-sensitive mask coating (e.g., silicone) that can be applied to regions of the layups 102ₐ, 102_{b} that will not be sealed (e.g., inner surfaces of the cavity). As such, an inner surface 113ₐ (FIG. 2) of the first layup 102ₐ and an inner surface 113_{b} (FIG. 2) of the second layup 102_{b} can collectively define an internal cavity 110 (FIG. 2) configured to accommodate a fluid (e.g., compressible, non-compressible). Because the first layup 102ₐ, the second layup 102_{b}, and the traces 104ₐ, 104_{b} of the circuit 100 may be fabricated from flexible materials, the circuit 100 of FIG. 1 can stretch as the fluid is introduced to the internal cavity. In other words, the circuit 100 of FIG. 1 can be selectively inflated and deflated as the fluid is introduced and/or removed from the internal cavity. According to the non-limiting aspect of FIG. 1, the fluid-fillable circuit 100 is depicted in an inflated condition, meaning the internal cavity defined by an inner surface 113ₐ (FIG. 2) of the first layup 102ₐ and an inner surface 113_{b} (FIG. 2) of the second layup 102_{b} is accommodating a fluid.

Still referring to FIG. 1, the circuit 100 can be inflated using various known methods of inflation, such as the method described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety. For example, the first layup 102ₐ and the second layup 102_{b} of FIG. 1 can include a multi-layer construction, and at least a portion of the first layup 102ₐ and the second layup 102_{b} can be fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 100 of FIG. 1, and fluid can be selectively introduced and/or removed from the internal cavity via the valve assembly.

Referring now to FIG. 2, a cross-sectioned view of either the fluid-fillable circuit 100 of FIG. 1 is depicted in accordance with at least one non-limiting aspect of the present disclosure. The cross-section of FIG. 2 was taken about line 1B in FIG. 1. In FIG. 2, the multi-layer nature of the first layup 102ₐ and the second layup 102_{b} becomes apparent. For example, according to the non-limiting aspect of FIG. 2, the layups 102ₐ, 1012_{b} can include a two-layer 112, 114 construction. Specifically, each of the first layup 102ₐ and the second layup 102_{b} of the fluid-fillable circuit 100 of FIG. 1 can include a substrate layer 112ₐ, 112_{b} and an encapsulation layer 114ₐ, 114_{b}. However, according to other non-limiting aspects, the layups 102ₐ, 102_{b} can include three or more layers, including a stencil layer configured to accommodate the traces 104_{a,b} (FIG. 1), as described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. In still other non-limiting aspects, the layups 102ₐ, 102_{b} can include a single layer configured to accommodate the traces 104ₐ, 104_{b}.

Alternately and/or additionally, the flexible structures described in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety, can be used to fabricate one or more layers of the layups 102ₐ, 102_{b}, as depicted in FIG. 2. For example, the layups 102ₐ, 102_{b} can be produced from one or more layers including a B-stage resin film, a C-stage resin film, an adhesive, a TPU, and/or a silicone material, amongst others.

According to the non-limiting aspect of FIG. 2, the substrate layers 112ₐ, 112_{b} of the first and second layups 102ₐ, 102_{b} can include one or more features, such as a contact point configured to mechanically and/or electrically engage with an electronic component mounted to the layups 102ₐ, 102_{b} and/or one or more of the traces 104_{a,b}. The traces 104_{a,b} and, more specifically, a deformable conductor from which the traces 104_{a,b} are composed, can be deposited either on or embedded within a portion of the substrate layers 112ₐ, 112_{b}. Collectively, the encapsulation layers 114ₐ, 114_{b} can contain and protect the fluid-fillable circuit 100, including any traces 106, electronic components, and/or contact points coupled to the substrate layers 112ₐ, 112_{b}. The encapsulation layers 114ₐ, 114_{b} can also fill any spaces between the components and the substrate layers 112ₐ, 112_{b}. For example the encapsulation layer 114 can be formed from materials suitable for the encapsulation of electronics, including silicone-based materials such as PDMS, urethanes, epoxies, polyesters, polyamides, and/or varnishes, amongst other materials capable of providing a sufficient protective coating and/or assisting in holding the fluid-fillable circuit 100 assembly together.

According to some non-limiting aspects, the circuit 100 of FIG. 2 can be assembled in accordance with the design for manufacture techniques disclosed in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety. For example, the traces 104ₐ, 104_{b}, vias 106ₐ, 106_{b}, and contacts (not shown) may be particularly sized and spaced, the ampacity of traces 104ₐ, 104_{b} may be configured, and the various features of the layups 102ₐ, 102_{b} may be attached in accordance with the techniques described for non-inflatable laminate structures, as disclosed therein.

For example, the substrate layers 112ₐ, 112_{b} and encapsulation layers 114ₐ, 114_{b} can be configured similar to the substrate layers and encapsulants described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. Alternatively and/or additionally, the substrate layers 112ₐ, 112_{b} and encapsulation layers 114ₐ, 114_{b} can be formed from a microlayer membrane that facilitates inflation of the fluid-fillable circuit 100 with a compressible fluid, such as the membranes disclosed in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety.

As depicted in FIG. 2, one or more vias 106 can be configured to traverse at least a portion of the substrate layers 112ₐ, 112_{b} and/or the encapsulation layers 114ₐ, 114_{b} of the layups 102ₐ, 102_{b}, creating an electrical conduit by which a desired electrical connection between electrical features of the first and second layups 102ₐ, 102_{b} can be established. For example, according to the non-limiting aspect of FIG. 2, the vias 106 can be formed through the substrate layers 112ₐ, 112_{b} such that the traces 104ₐ, 104_{b} of the first and second layups 102ₐ, 102_{b} are electrically coupled. However, vias 106 can similarly be configured to electrically couple electronic components and/or contact points coupled to each of the first and second layups 102ₐ, 102_{b}. According to other non-limiting aspects, the top layup 102ₐ and the bottom layup 102_{b} can each be independently encapsulated, such that each surface of the substrate layer 112ₐ of the top layup 102ₐ and the substrate layer 112_{b} of the bottom layup 102_{b} is covered by the encapsulation layer 114ₐ of the top layup 102ₐ and the encapsulation layer 114_{b} of the bottom layup 102_{b}, respectively.

In such aspects, one or more vias 106 may traverse the encapsulation layers 114ₐ, 114_{b}, such that a trace 106ₐ on the substrate layer 112ₐ of the top layup 112ₐ is electrically coupled to a trace 106_{b} on the substrate layer 112_{b} of the bottom layup 102_{b}.

According to other non-limiting aspects, the layups 102ₐ, 102_{b} can further include a stencil configured to accommodate the traces 104ₐ, 104_{b}, such as those described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. For example, a stencil can be particularly constructed to define paths for one or more of the traces 104ₐ, 104_{b} that traverse the fluid-fillable circuit 100 in accordance with a schematic for the fluid-fillable circuit 100. The paths defined by such stencils can accommodate the deformable conductor, which can be deposited within the stencil and covered by the encapsulation layers 114ₐ, 114_{b} such that the stencil and deformable conductors, which define the traces 104ₐ, 104_{b}, are bound between the substrate layers 112ₐ, 112_{b} and encapsulation layers 114ₐ, 114_{b}. Optionally, a pattern of traces may be formed in or on the substrate layers 112ₐ, 112_{b}, e.g., by laser ablation, thermoforming, molding, or other suitable additive or subtractive methods prior to depositing the deformable conductor on or in the substrate layer.

Still referring to FIG. 2, the internal cavity 110 collectively defined by the inner surface 113ₐ of the first layup 102ₐ, the inner surface 113_{b} of the second layup 102_{b}, and the seal 108 is illustrated in accordance with at least one non-limiting aspect of the present disclosure. As noted in reference to FIG. 1, the internal cavity 110 can accommodate a fluid (e.g., octafluoropropane, nitrogen, air, etc.), which can be compressible or non-compressible. The fluid can be pressurized as required by the particular application. For example, it may be desirable to pressurize the internal cavity 110 to a gauge pressure within a range of 1-35 pounds-per-square-inch (“PSI”). According to some non-limiting aspects, the internal cavity 110 may be configured to accommodate a pressure of about 20 PSI in an inflated condition. In other words, the fluid-fillable circuit 100—and more specifically, the internal cavity 110 collectively defined by the inner surface 113ₐ of the first layup 102ₐ, the inner surface 113_{b} of the second layup 102_{b}, and the seal 108—can be particularly configured to accommodate an internal fluid pressure that is slightly above ambient, or relatively high. In still other non-limiting aspects, the fluid-fillable circuit 100 can be similarly configured to bladders made using aspects described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety.

As depicted in FIG. 2, the fluid-fillable circuit 100 can be geometrically configured such that the internal cavity 110 includes a stadium-like (e.g., substantially rectangular, with rounded edges) cross section. However, the fluid-fillable circuit 100 can be alternately configured to have a number of different geometries, depending on user preference and/or intended application. For example, according to some non-limiting aspects, the fluid-fillable circuit 100 can be circular, spherical, hexagonal, rectangular, triangular or irregularly shaped. Such configurations may not be apparent from the cross-section of the fluid-fillable circuit 100, but evident when the fluid-fillable circuit 100 is viewed from above. In still other non-limiting aspects, the fluid-fillable circuit 100 can have an arbitrary or abstract geometry, such as the bladders described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety. Such bladders are generally hexagonal but include one or more indents that abstract the geometry. According to other non-limiting aspects, the fluid-fillable circuit 100 can be one of a plurality of inflatable circuits 100, wherein the plurality of inflatable circuits 100 are arranged in a tessellated pattern. In other words, each fluid fillable circuit 100 of the plurality can have a substantially similar shape and can be arranged such that the plurality of inflatable circuits 100 collectively cover an area without a significant gap and/or overlap.

Alternately and/or additionally, a fluid-fillable circuit 100 may be formed such that the circuit 100 is substantially flat in an uninflated configuration but can be molded to have varied three-dimensional surface topographies and shapes with varying degrees of complexity and contour. According to such aspects, three-dimensional forming can occur at the time of inflation, for example by thermoforming in a mold cavity having the desired final three-dimensional shape using, for example, inflation pressure. According to other non-limiting aspects, a fluid-fillable circuit 100 or system may be similarly molded in a secondary operation post-inflation, for example by thermal compression-molding in a mold cavity having the desired final 3-dimensional shape. It shall be appreciated that such methods of formation can be employed to form any of the circuits disclosed herein.

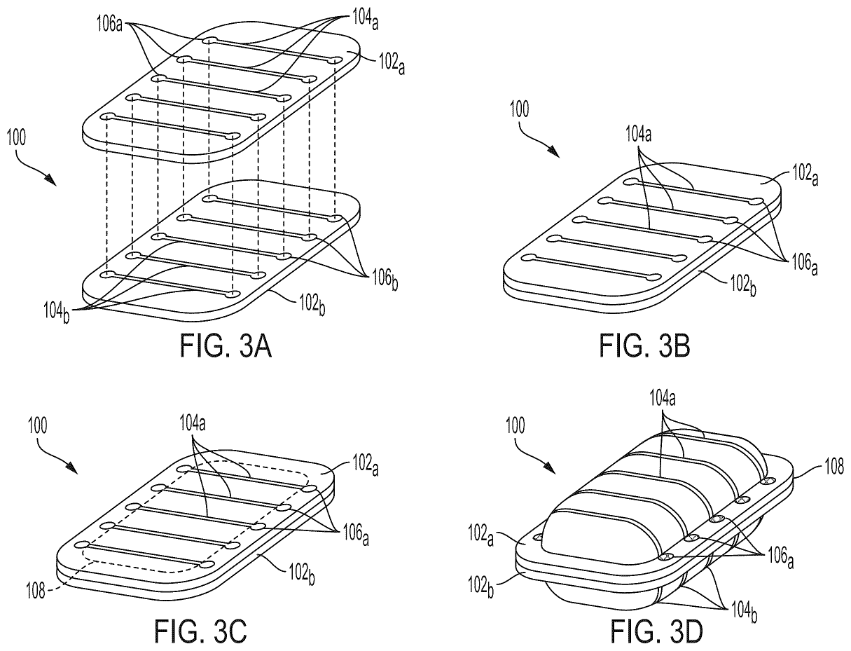

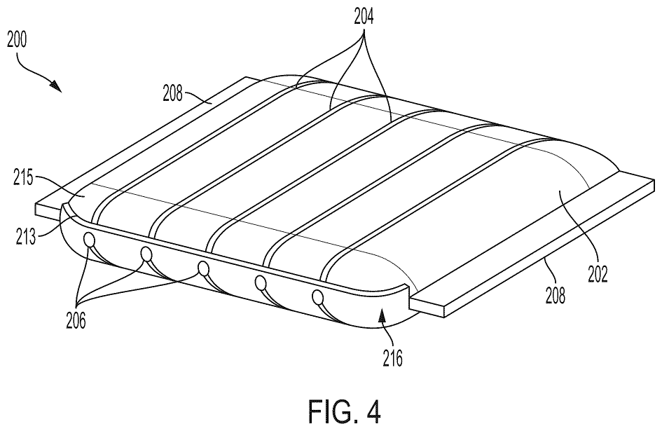

Referring now to FIGS. 3A-3D, several assembly diagrams of the fluid-fillable circuit 100 of FIG. 1 are depicted in accordance with at least one non-limiting aspect of the present disclosure. Specifically, FIGS. 3A-3D illustrate how the first layup 102ₐ can include features (e.g., traces 104ₐ, vias 106ₐ, etc.) that can be particularly dimensioned and positioned on the first layup 102ₐ, such that they correspond to features (e.g., traces 104_{b}, vias 106_{b}, etc.) on the second layup 102_{b}. For example, as depicted in FIG. 3A, the traces 104ₐ and vias 106ₐ of the first layup 102ₐ are dimensioned and positioned on the first layup 102ₐ such that they align with and electrically coupled to a corresponding trace 104 or via 106_{b} on the second layup 102_{b}. This alignments is depicted in FIG. 3B, where the first layup 102ₐ is positioned above the second layup 102_{b}, such that corresponding features of the first and second layup 102ₐ, 102 can be electrically coupled.

According to the non-limiting aspect of FIG. 3B, the first layup 102ₐ can have a design that is substantially similar (e.g., a one-for-one match) to a design of the second layup 102_{b}. In other words, the features of the first layup 102ₐ can be similarly dimensioned and positioned relative to corresponding features of the second layup 102_{b}. However, according to other non-limiting aspects, the first layup 102ₐ can be alternately designed relative to the second layup 102_{b}. Some or all of the features (e.g., traces 104ₐ, vias 106ₐ, etc.) of the first layup 102ₐ can be alternately dimensioned and/or positioned relative to corresponding features on the second layup 102_{b}. For example, it might be desirable to account for manufacturing tolerances and/or alignment issues by dimensioning one or more of the vias 106ₐ of the first layup 102ₐ with a larger diameter relative to a diameter of a corresponding via 106_{b} of the second layup 102_{b}. Alternately and/or additionally, one or more of the vias 106ₐ of the first layup 102ₐ can be positioned such that it is staggered relative to a corresponding via 106_{b} of the second layup 102_{b}. Such design modifications can ensure a proper electrical connection between traces 104ₐ, 104_{b} can be achieved. In still other non-limiting aspects, the features of the layups 102ₐ, 102_{b} have different geometric configurations (e.g., triangular, rectangular, hexagonal, three-dimensional, etc.).

According to still other non-limiting aspects, the layups 102ₐ, 102_{b} of FIGS. 3A-3D can have differing sizes and/or shapes. For example, according to some non-limiting aspects, the second layup 102_{b} can have a larger overall surface area relative to the first layup 102ₐ, or vice-versa, such that only a portion of the circuit 100 is inflatable and some features (e.g., traces 104_{b}, vias 106_{b}, etc.) of the second layup 102_{b} are do not correspond to the shape of the internal cavity 110 (FIG. 2). Layups 102ₐ, 102_{b} of differing sizes and/or shapes can be useful for aspects in which the fluid-fillable circuit 100 is integrated into an external structure or housing, wherein only a portion of the structure or housing is intended to support inflation of the circuit 100, but where a stretchable, bendable, or otherwise flexible circuit is integrated to other portions.

Additionally and/or alternately, such non-inflated circuit portions can be in electrical communication with inflated portions of the fluid-fillable circuit 100. Furthermore, both the first and second layups 102ₐ, 102_{b} can have portions that do not correspond to the shape (e.g., when viewed in plan, for example) of the internal cavity 110, and in some non-limiting aspects, such portions may overlap and/or substantially overlay one another. According to still other non-limiting aspects overlapping or overlaid portions can be bonded or otherwise unitized to one another. In all such examples where at least one uninflated portion does not correspond to the shape of the internal cavity is provided in either and/or both of the first and second layups 102ₐ, 102_{b}, such uninflated portions may extend from a surface of the inflated portion of the circuit 100 and/or the seal 108, itself. Such uninflated portions may extend into the cavity 110 so as to be contained within the internal volume of the inflated circuit, or they may extend away from the inflated portion such that they are external to the volume of the cavity 100. It shall be appreciated that similar uninflated portions can be implemented in any of the circuits disclosed herein.

As such, it shall be appreciated that the geometric configuration of the layups 102ₐ, 102_{b} of FIGS. 3A-3D, including the geometric configuration of their respective features (e.g., traces 104ₐ, 104_{b}, vias 106ₐ, 106_{b}, etc.) are not intended to be limiting, and that the present disclosure contemplates numerous aspects wherein the geometric configuration of the layups 102ₐ, 102_{b} are attenuated to achieve a desired mechanical and/or electrical integration of the fluid-fillable circuit 100.

According to FIG. 3C, once the layups 102ₐ, 102_{b} are aligned, as depicted in FIG. 3B, features (e.g., traces 104ₐ, vias 106ₐ, etc.) of the first layup 102ₐ can be electrically coupled to corresponding features (e.g., traces 104_{b}, vias 106_{b}, etc.) of the second layup 102_{b}. For example, in FIG. 3C, the substrate layer 112ₐ of the top layup 102ₐ can be brought into contact with or otherwise positioned adjacent the substrate layer 112_{b} of the bottom layup 102_{b}, such that the vias align 106ₐ, 106_{b} and corresponding traces 104ₐ of the top layup 102ₐ are in electrical communication with the traces 104_{b} of the bottom layup 102_{b}. It shall be appreciated that in embodiments having layups that are mirror images of one another, such as the ones depicted here, it may be desirable that corresponding features such as vias are formed in the same layer of both layups (e.g., the encapsulation layers, or the substrate layers) for ease of manufacturing and inventory control. However, in embodiments (not shown here) where the various layups are not mirror images of one another, it may be preferable but not necessary for corresponding features such as vias to be formed in different layers of each layup (e.g., the encapsulation layer of one and the substrate layer of the other). A region of the first layup 102ₐ can be subsequently bonded or unitized to a corresponding region of the second layup 102_{b} by any known process of attachment (e.g., welding, soldering, fusing, stitching, adhesives, etc.), thereby creating the seal 108 of the internal cavity 110 (FIG. 2). According to the non-limiting aspect of FIG. 3C, the corresponding regions—and thus, the sealed portion—of the first and second layups 102ₐ, 102_{b} can be located on the perimeter of the first and second layups 102ₐ, 102_{b}, and can include any overlapping vias 106ₐ, 106_{b} positioned in those regions. As such, the first and second layups 102ₐ, 102_{b} can be securely fastened, and the traces 104ₐ, 104_{b} reliably held in electrical communication by the aligned vias 106ₐ, 106_{b}. Accordingly, the fluid-fillable circuit 100 can be assembled such that a portion of the inner surface 113ₐ (FIG. 2) of the first layup 102ₐ is not adhered to a portion of the inner surface 113_{b} (FIG. 2) of the second layup 102_{b} and thus, the portion of the inner surface 113ₐ (FIG. 2) can be separated from the portion of the inner surface 113_{b} (FIG. 2). Such separation can be caused by a fluid inserted into the internal cavity 110 (FIG. 2) and contained within the internal cavity 110 (FIG. 2) via the seal 108. Thus, due to the deformable nature of the substrate layers 112ₐ, 112_{b}(FIG. 2), encapsulation layers 114ₐ, 114_{b} (FIG. 2), and traces 104ₐ, 104_{b}, the circuit 100 can be inflated, as illustrated in FIG. 3D. It may be appreciated that after sealing, portions of the seal 108 may not be structurally or electrically necessary and may be trimmed from the fluid-fillable circuit 100 for aesthetic or other reasons and that this is true for all of the circuits disclosed herein.

Referring now to FIG. 4, a perspective view of another inflatable circuit 200 is depicted in accordance with at least one non-limiting aspect of the present disclosure. According to the non-limiting aspect of FIG. 4, the fluid-fillable circuit 200 can include a single layup 202 with a plurality of traces 204. The single layup 202 of FIG. 4 can include a multi-layer construction, similar to the layups 102ₐ, 102_{b} of FIG. 2. Likewise, each trace 204 of the plurality can be constructed from a deformable conductive material deposited on or within a substrate layer and encapsulated. According to some non-limiting aspects, the layup 202 can further include a stencil, which can define paths for the traces 204 in which the deformable conductor can be deposited and contained.

According to the non-limiting aspect of FIG. 4, the layup 202 can be folded or rolled such that a first portion of the layup 202 overlaps with a corresponding, second portion of the layup 202. The layup 202 can be geometrically configured such that, when the layup 202 is folded, one or more vias 206 positioned on the first portion of the layup 202 can be aligned and placed in electrical communication with a corresponding via 206 the second portion of the layup 202. The alignment of the vias 206 in this embodiment results in a desired alignment of traces 204, which traverse between a via 204 on the first portion and a via 206 on the second portion. In some examples, traces 204 may have patterns that do not overlay as shown. Once the vias 206 and traces 204 are aligned, the first portion of the layup 202 can be bonded to the second portion of the layup 202 by any known process of attachment (e.g., welding, soldering, fusing, stitching, adhesives, etc.), thereby creating a sealed lap joint 216 between an inner surface 213 and an outer surface 215 of the layup 202. According to the non-limiting aspect where the single layup 202 has a construction similar to the layups 102ₐ, 102_{b} of FIG. 2, an encapsulation layer of the layup 202 can overlap and be bonded to a substrate layer of the layup 202 to form the lap joint 216. The creation of the lap joint 216 may result in non-overlapping, side portions of the layup 202, which may be bonded via any known process of attachment (e.g., welding, soldering, fusing, stitching, adhesives, etc.), thereby creating a seal 208 on either side of the fluid-fillable circuit 200. The seal 208 can be formed in the same bonding operation as the lap joint 216 or, alternately, via a separate bonding operation. According to some non-limiting aspects, the layup 202 can be alternately configured such that the otherwise additional lap joints can be formed on either side of the fluid-fillable circuit 200, such that the additional lap joints can be used in lieu of the seals 208 of FIG. 4.

Similar to the multi-layup circuit 100 of FIG. 2, when assembled, the circuit 200 of FIG. 4 can define an internal cavity configured to accommodate and contain a fluid. Specifically, the internal cavity can be defined by the inner surface 213, the lap joint 216, and the seal 208 of the layup 202. Although the circuit 200 of FIG. 4 defines an internal cavity having a stadium-like cross section, the fluid-fillable circuit 200 can be alternately configured to have a number of different geometries, depending on user preference and/or intended application. For example, according to some non-limiting aspects, the fluid-fillable circuit 200 can be circular, hexagonal, rectangular, or triangular. Similar to the circuit 100 of FIG. 1, according to other non-limiting aspects, the circuit 200 of FIG. 4 is one of a tessellated plurality. Regardless of its particular geometric configuration, the circuit 200 of FIG. 4 can be inflated by inserting a fluid into the internal cavity defined by the inner surface 213, the lap joint 216, and the seal 208. Notably, FIG. 4 depicts the circuit 200 in an inflated condition.

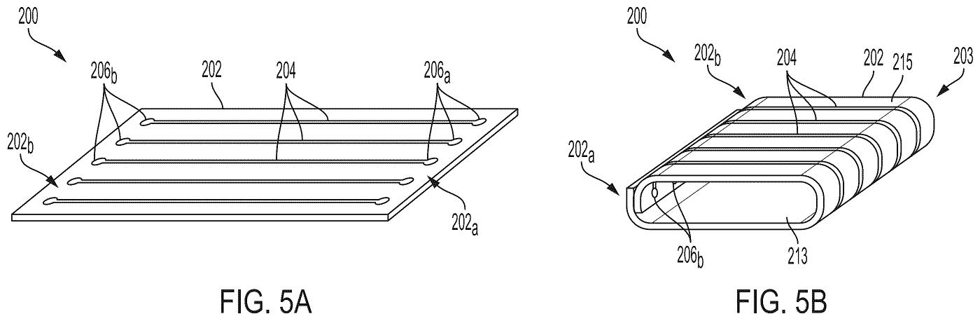

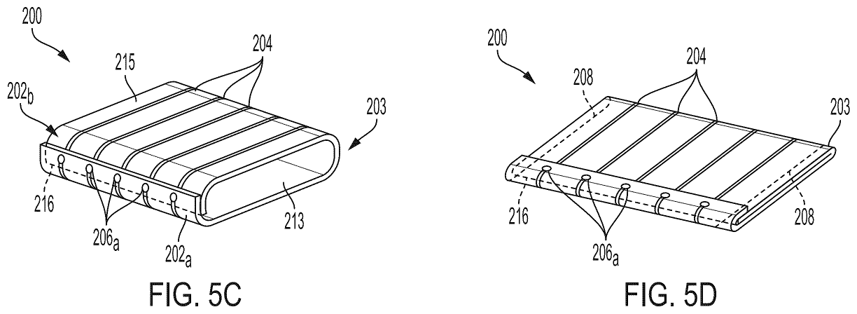

Referring now to FIGS. 5A-5E, several assembly diagrams of the fluid-fillable circuit 200 of FIG. 4 are depicted in accordance with at least one non-limiting aspect of the present disclosure. Specifically, FIG. 5A illustrates how the layup 202 can have a first portion 202ₐ and a second portion 202_{b}. A first plurality of vias 206ₐ can be positioned on the first portion 202ₐ and a second plurality of vias 206_{b} can be positioned on the second portion 202_{b}. Each trace 204 from the plurality of traces 204 can be electrically coupled to corresponding vias 206ₐ, 206_{b} and thus, configured to traverse the layup 202 from the first portion 202ₐ to the second portion 202_{b}.

In reference to FIG. 5B, a fold 203 in the layup 202 is illustrated such that the first portion 202ₐ of the layup 202 is aligned with the second portion 202_{b}. More specifically, the vias 206ₐ (not shown) of the first portion 202ₐ are aligned with the vias 206_{b} of the second portion 202_{b}. After alignment, the vias 206ₐ (not shown) of the first portion 202ₐ can be electrically coupled to the vias 206_{b} of the second portion 202_{b}. Moreover, the alignment of FIG. 5B prepares the layup 202 for the bonding procedure that will result in the lap joint 216. For example, the fold 203 in the layup 202 is particularly configured such that an inner surface 213 of the first portion 202ₐ of the layup 202 can mechanically engage an outer surface 215 of the second portion 202_{b} of the layup 202.

Accordingly, the layup 202 of FIG. 5B is properly aligned and prepared for the bonding process, as depicted in FIG. 5C. The first portion 202ₐ of the layup 202 can be bonded to the second portion 202_{b} of the layup 202 by any known process of attachment (e.g., welding, soldering, fusing, stitching, adhesives, etc.), thereby creating the lap joint 216. The process can further secure the electrical connection of the vias 206ₐ on the first portion 202ₐ of the layup 202 to the vias 206_{b} on the second portion 202_{b} of the layup 202. Since, each trace 204 is electrically coupled to corresponding vias 206ₐ, 206_{b}, when each pair of corresponding vias 206ₐ, 206_{b} are electrically coupled, that part of the circuit 200 (e.g., first via 206ₐ, second via 206_{b}, and the connecting trace 204) is closed and mechanically secured to ensure a robust electrical connection.

Referring now to FIG. 5D, after the lap joint 216 is formed, the circuit 200 can be flattened along the fold 203, such that a majority of the inner surface 213 of the layup 202 is faces, but is not adhered to another portion of the inner surface 213 of the layup 200. Accordingly, a seal 208 may be formed on either side of the circuit, wherein each seal 208 is perpendicular to the lap joint 216. The seals 208 can be formed by any known process of attachment (e.g., welding, soldering, fusing, stitching, adhesives, etc.) and this process can be done in conjunction with, or separate from, the creation of the lap joint 216. One or more notches can be introduced into the layup 202, prior to the formation of the seals 208, to reduce mechanical interference during inflation.

Accordingly, the fluid-fillable circuit 200 can be assembled such that a majority of the inner surface 213 of the layup 202 is folded such that it faces—but is not adhered to—another portion of the inner surface 213 of the layup 200 and thus, forms an internal cavity of the fluid-fillable circuit 200. A fluid can be inserted into the internal cavity formed by the inner surface 213, lap joint 216, and seals 208 of the layup 200 and contained. As more fluid is inserted within the internal cavity, the more pressure will be exerted on the inner surfaces 213 of the layup 202. Due to the deformable nature of the layup 202 and traces 202, the circuit 200 can be inflated, as illustrated in FIG. 5E.

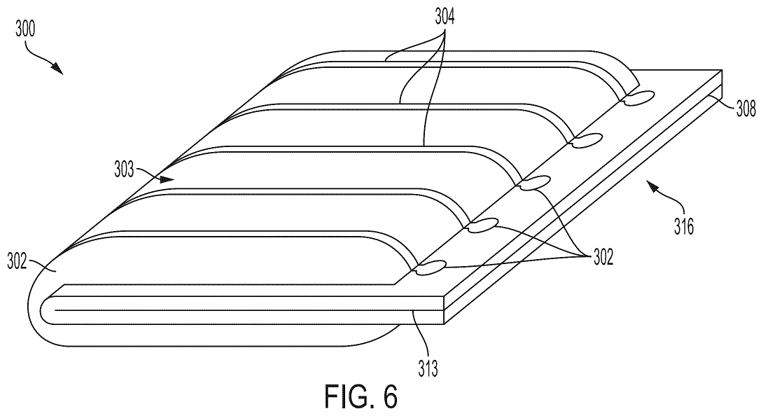

Referring now to FIG. 6, a perspective view of another inflatable circuit 300 is depicted in accordance with at least one non-limiting aspect of the present disclosure. Similar to the circuit 200 of FIG. 4, the circuit 300 of FIG. 6 can include a single layup 302 construction, wherein the single layup 302 can include a plurality of traces 304 and a plurality of vias 306. Once again, the single layup 302 of FIG. 6 can include a multi-layer construction, similar to the layups 102ₐ, 102_{b} of FIG. 2. Likewise, each trace 304 of the plurality can be constructed from a deformable conductive material deposited on or within a substrate layer and encapsulated. According to some non-limiting aspects, the layup 302 can further include a stencil layer, which can define paths for the traces 304 in which the deformable conductor can be deposited and contained.

Similar to the circuit 200 of FIG. 4, the layup 302 can be folded such that a first portion of the layup 302 interfaces with a corresponding, second portion of the layup 302. The layup 302 can be geometrically configured such that, when the layup 302 is folded or rolled, one or more vias 306 positioned on the first portion of the layup 302 can be aligned and placed in electrical communication with a corresponding via 306 the second portion of the layup 302. The alignment of the vias 306 may result in a desired alignment of traces 304 as shown in this embodiment, or traces may be misaligned or otherwise have an asymmetric configuration. As shown here, the vias traverse linearly and in alignment between a via 304 on the first portion and a via 304 on the second portion. However, according to the non-limiting aspect of FIG. 6, the circuit 300 can be folded or rolled such that every portion of an inner surface 313 of the layup 302 faces another portion of the inner surface 313 of the layup. In other words, no portion of the layup 302 of FIG. 6 overlaps with another portion and thus, the circuit 300 of FIG. 6 does not have a lap joint 216 (FIG. 4), such as the circuit 200 of FIG. 4. Rather, once the vias 306 and traces 304 are aligned, a seal 308 can be formed between inner surfaces 313 of the first and second portions of the layup 302 by any known process of attachment (e.g., welding, soldering, fusing, stitching, adhesives, etc.). In an alternative embodiment (not shown), the outer surface 315 may be folded or rolled over at a free edge of the layup 302, e.g., the edge comprising vias 306, such that the outer surfaces 315 contact one another, and the seal 308 formed adjacent this edge such that the seal 308 is contained within the cavity defined within the interior surface 313 of the layup 302. The remaining edges, e.g., the remaining two edges at opposite ends of the circuit 300, may be sealed as shown here. In another aspect (not shown), two free edges may be folded or rolled over such that the outer surfaces may contact one another, and the seal 308 formed adjacent these edges such that the seal is contained between the interior surface 313. The remaining edge(s), e.g., the remaining edges comprising the vias of the circuit 300, may be sealed as shown here. According to the non-limiting aspect where the single layup 302 has a construction similar to the layups 102ₐ, 102_{b} of FIG. 2, an encapsulation or substrate layer of the layup 302 can be overlaid onto itself such that vias 306 positioned on the first portion of the layer can be aligned with and electrically coupled to a corresponding via 306 positioned on the second portion of the encapsulation layer. The seal(s) 308 may comprise the overlaid vias, thereby providing reliable mechanical and electrical coupling of the vias to one another.

According to the non-limiting aspect of FIG. 6, the seal 308 can create an edge joint 316 that, along with the fold 303, can form an internal cavity similar to the internal cavity 110 of FIG. 2. Similar to the multi-layup circuit 100 of FIG. 2, when assembled, the internal cavity defined by the circuit 300 of FIG. 6 can accommodate and contain a fluid. Although the circuit 300 of FIG. 6 defines an internal cavity having a stadium-like cross section, the fluid-fillable circuit 300 can be alternately configured to have a number of different geometries, depending on user preference and/or intended application. For example, according to some non-limiting aspects, the fluid-fillable circuit 300 can be circular, hexagonal, rectangular, or triangular. Similar to the circuit 100 of FIG. 1, according to other non-limiting aspects, the circuit 300 of FIG. 6 is one of a tessellated plurality. Regardless of its particular geometric configuration, the circuit 300 of FIG. 6 can be inflated by inserting a fluid into the internal cavity defined by the inner surface 313, the edge joint 316, and the fold 303. Notably, FIG. 6 depicts the circuit 300 in an inflated condition. According to other non-limiting aspects, a butt joint can be utilized to seal the circuit 300 about a thickness of the layup 302, in lieu of the edge joint 316 of FIG. 6.

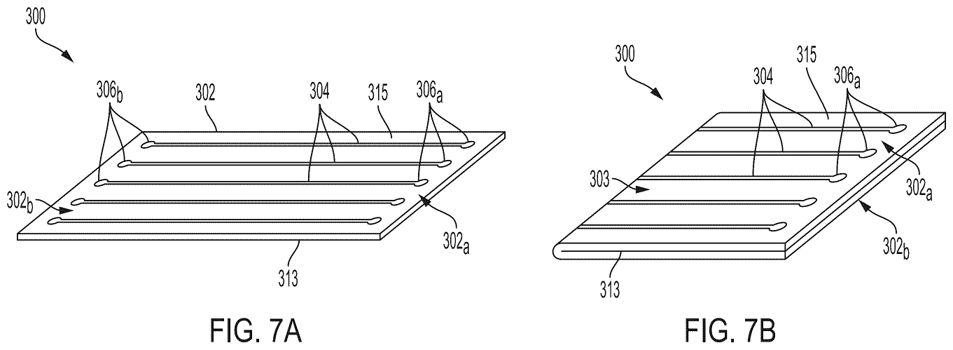

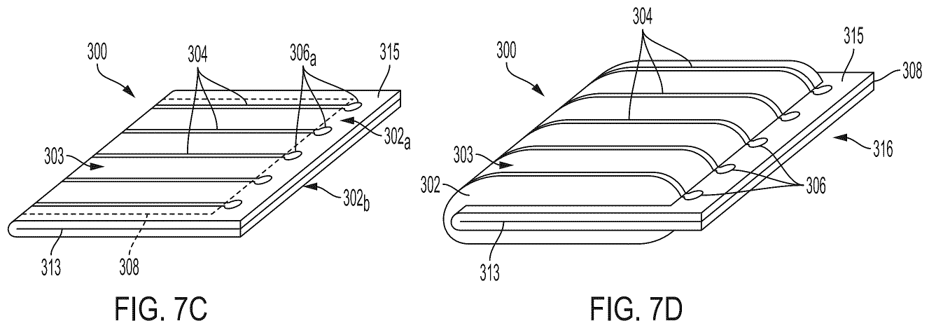

Referring now to FIGS. 7A-7D, an assembly of the fluid-fillable circuit 300 of FIG. 6 is depicted in accordance with at least one non-limiting aspect of the present disclosure. Specifically, FIG. 7A illustrates how the layup 302 can have a first portion 302ₐ and a second portion 302_{b}. A first plurality of vias 306ₐ can be positioned on the first portion 302ₐ and a second plurality of vias 306_{b} can be positioned on the second portion 302_{b}. Each trace 304 from the plurality of traces 304 can be electrically coupled to corresponding vias 306ₐ, 306_{b} and thus, configured to traverse the layup 302 from the first portion 302ₐ to the second portion 302_{b}.

In reference to FIG. 7B, a fold 303 in the layup 302 is illustrated such that the first portion 302ₐ of the layup 302 is aligned with the second portion 302_{b}. According to the non-limiting aspect of FIG. 7B, the circuit 300 can be folded such that the edges of the portions 302ₐ and 302_{b} are aligned. As shown in this aspect, the inner surface 313 of the first portion 302ₐ of the layup 302 faces the inner surface 313 of the second portion 302_{b} of the layup 302. The vias 306ₐ of the first portion 302ₐ of the layup 302 can be aligned with the vias 306_{b} (not shown) of the second portion 302_{b} of the layup 302. After alignment, the vias 306ₐ of the first portion 302ₐ can be electrically coupled to the vias 306_{b} (not shown) of the second portion 302_{b}. Moreover, the alignment of FIG. 7B prepares the layup 302 for the bonding procedure that will result in an edge joint 316.

Accordingly, the layup 302 of FIG. 7B is properly aligned and prepared for the bonding process, as depicted in FIG. 7C. A seal 308 can be formed between the first portion 302ₐ and the second portion 302_{b}, as shown here at the inner surface 313, by any known process of attachment or unitizing (e.g., welding, soldering, fusing, stitching, adhesives, etc.). The seal 308 of this embodiment creates an edge joint 316 between the first and second portions 302ₐ, 302_{b} that comprises the vias 306ₐ, 306_{b}. The process as shown may thus mechanically secure the electrical connection of the vias 306ₐ to the vias 306_{b}. Since each trace 304 is electrically coupled at corresponding vias 306ₐ, 306_{b}, when each pair of corresponding vias 306ₐ, 306_{b} are electrically coupled that part of the circuit 300 (e.g., first via 306ₐ, second via 306_{b}, and the connecting trace 304) is closed and mechanically secured to ensure a robust electrical connection. It should be appreciated that a seal may be provided in a portion (not shown) of the circuit 300 that does not contain vias, and optionally the seal may not be located at free edges of the circuit 300 thereby providing uninflated portions (not shown) that extend beyond the seal. The seal may comprise traces and thus, traces from an inflated portion may be in electrical communication with uninflated portions of the circuit 300. It may be appreciated that vias may be located in any permutation of inflated portions, seals or joints, or uninflated portions of the circuit 300. Vias that connect one trace end to another trace end may provide open or closed electrical communication in a single trace or between different traces of the circuit 300, to an electronic component, or to another inflated or uninflated circuit.

In further reference to FIG. 7C, after the edge joint 316 is formed, a substantial portion of the inner surface 313 of the layup 302 can face—but is not adhered to—another portion of the inner surface 313 of the layup 300. Accordingly, the seal 308, which can be formed on every side of the circuit 300 that is not bound by the fold 303, can form an internal cavity (e.g., similar to the internal cavity 110 of FIG. 2) of the fluid-fillable circuit 300. A fluid can be inserted into and contained by the internal cavity formed by the inner surface 313, edge joint 316, and fold 303. As more fluid is inserted within the internal cavity, the more pressure will be exerted on the inner surfaces 313 of the layup 302. Due to the deformable nature of the layup 302 and traces 304, the circuit 300 can be inflated, as illustrated in FIG. 7D.

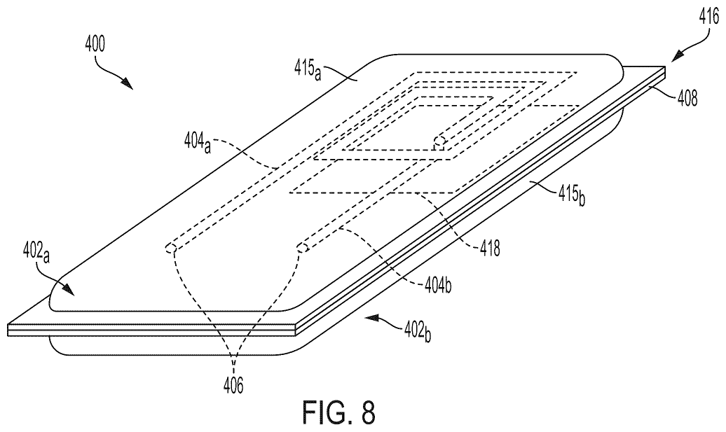

Referring now to FIG. 8, a perspective view of another inflatable circuit 400 is depicted in accordance with at least one non-limiting aspect of the present disclosure. According to the non-limiting aspect of FIG. 8, the traces 404ₐ, 404_{b} of the fluid-fillable circuit 400 can be particularly configured such that the fluid-fillable circuit can perform a variety of electrical functions. In other words, whereas the previously discussed aspects of FIGS. 1-7 described several non-limiting mechanical constructions and interconnects between traces of several inflatable circuit embodiments, the non-limiting aspect of FIG. 8 demonstrates a fluid-fillable circuit 400 electrically configured to perform an intended function, which can vary depending on the intended application.

Specifically, an electrical current and/or potential can be applied to the circuit 400, e.g., at vias 406 and, when the circuit 400 is inflated, an electrical parameter may be generated (e.g., an inductance, a resistance, a voltage drop, a capacitance, signal, an electromagnetic field, etc.) that is associated with the trace pattern configuration and applied current and/or potential.

According to some non-limiting aspects, electronic components (e.g., a power source, a microprocessor, a logic-based controller, a transceiver, an electrode, a light emitting diode (“LED”) bank, a pump etc.) can be mechanically secured to and/or otherwise integrated with the layups 402ₐ, 402_{b} and/or electrically coupled to the traces 404ₐ, 404_{b} of the circuit 400. The electronic components can be configured to receive and utilize signals from the traces 404ₐ, 404_{b} and electrical parameters from the traces 404ₐ, 404_{b} in accordance with the intended application. The electrical parameters and signals may vary with a physical change to the fluid-filled circuit 400, for example the inflation pressure, volume of the cavity, application of an external stimulus and/or deformation of the circuit 400. The resulting changes to the electrical parameters may be monitored, transmitted, or otherwise utilized to dynamically or statically calculate, infer or otherwise determine one or more physical or structural characteristics or conditions of the circuit 400, and/or stimuli applied to the circuit 400.

According to the non-limiting aspect of FIG. 8, the fluid-fillable circuit 400 can include a first layup 402ₐ and a second layup 402_{b}, which can be positioned, coupled, and sealed together in accordance with the fluid-fillable circuit 100 of FIG. 1. Specifically, the first layup 402ₐ can be coupled to the second layup 402_{b} via a seal 408 between inner surfaces (not shown) of the first and second layup 402ₐ, 402_{b}, thereby forming an edge joint 416 between the two layups 402ₐ, 402_{b}. The first layup 402ₐ, the second layup 402_{b}, the seal 408, and the edge joint 416 can collectively define an internal cavity of the fluid-fillable circuit 400, similar to the internal cavity 110 of FIG. 2. The internal cavity can be configured to accommodate and contain a fluid. However, it shall be appreciated that the present disclosure contemplates other aspects in which other joints may be used to couple the first and second layup 402ₐ, 402_{b}. According to other aspects, the fluid-fillable circuit 400 of FIG. 8 can be made from a single layup using the techniques described in reference to FIGS. 4 and 6 and the associated descriptions thereof. Substrate layers of the first layup 402ₐ and second layup 402_{b} can have an outer surface 415ₐ, 415_{b} on which one or more traces 404ₐ, 404_{b} made from a deformable conductor can be deposited and encapsulated.

In further reference to FIG. 8, the traces 404ₐ can form a multi-layer or multi-level coil in a layup, e.g., the layup 402ₐ. The coiled trace 404ₐ can have a depth, meaning the coils descend towards the substrate layer of the layup 402ₐ, in some embodiments forming an Escher-like staircase configuration. The coil may be constructed using the principles disclosed in U.S. Provisional Application No. 63/270,589, titled FLEXIBLE THREE-DIMENSIONAL ELECTRONIC COMPONENT, filed Oct. 22, 2021 the disclosure of which is hereby incorporated by reference in its entirety. The trace 404ₐ can be specifically configured to generate a desired electrical parameter when an electric current and/or voltage is applied to the circuit 400. For example, since the trace 404ₐ of FIG. 8 is arranged in a coiled configuration, a current may generate a particular inductive and/or electromagnetic response as it is applied to the circuit 400 and traverses the trace 404ₐ. For example, an electromagnetic field may be generated by a current applied to the coil. However, other electrical parameters, such as an electrical resistance and/or a voltage drop across the trace 404ₐ may be simultaneously or separately generated and/or monitored as a current and/or potential is applied to the circuit 400 and the current traverses the trace 404ₐ.

Still referring to FIG. 8, a conductive layer 418, may be provided such as the conductors described in U.S. patent application Ser. No. 17/192,725, titled DEFORMABLE INDUCTORS, filed Mar. 4, 2021 and/or PCT Patent Application No. PCT/US2021/071374, titled WEARABLE ARTICLE WITH FLEXIBLE INDUCTIVE PRESSURE SENSOR, filed Sep. 3, 2021, the disclosures of both which are hereby incorporated by reference in their entirety. The conductive layer 418 can be mechanically coupled to an outer surface 415, an inner surface, or laid up in between any of the layers of the second layup 402_{b}. The conductive layer 418 can be generally positioned opposite the inductive coil formed by the trace 404ₐ on or within the first layup 402ₐ and can define a surface area that correlates to or that is large enough to fully encompass, or bound, all of the coils formed by the trace 404ₐ. Collectively, the coiled trace 404ₐ and conductive layer 418 can function as an inductive sensor configured to utilize inductive responses generated by the coiled trace 404ₐ to determine a relative distance between the coiled trace 404ₐ on the first layup 402ₐ and the conductive layer 418 on the second layup 402_{b}. The conductive layer 418 can be alternately and/or additionally configured to serve other functions, such as those described in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety. For example, the conductive layer 418 can be configured to simultaneously serve as an electromagnetic shield. Likewise, the conductive layer 418 can be manufactured from a conductive film and configured for use as a high-power bus, as needed. In other non-limiting aspects, the coiled trace 404ₐ is positioned on the bottom layup 402_{b} and the conductive layer 418 is positioned on the top layup 402ₐ, as long as they are opposing each other and the conductive layer 418 is large enough to encompass all of the coils formed by the trace 404ₐ.

According to some non-limiting aspects, one or more electronic components (e.g., a power source, a microprocessor, a logic-based controller, a transceiver, an electrode, a LED bank, a pump etc.) can be coupled to the circuit 400 via a trace 404ₐ, 404_{b} positioned on one of the layups 402ₐ, 402_{b}. Alternately and/or additionally, one or more electronic components can be coupled to one or more traces 406ₐ, 406_{b} of the layups 402ₐ, 402_{b}. The electronic component can be configured to receive and process signals from the coiled trace 404ₐ and/or the conductive layer 418. For example, the electronic component can be a microprocessor configured to correlate an inductive response generated by the coiled trace 404ₐ to relative distance between the coiled trace 404ₐ and the conductive layer 418, as will be further explained below. According to other non-limiting aspects, the electronic component can be a capacitor configured to create a resistor, an inductor, and a capacitor circuit configured to sense proximity of the coiled trace 404ₐ to the conductive layer 418.

Because the first layup 402ₐ, the second layup 402_{b}, and the traces 404ₐ, 404_{b} can be fabricated from flexible and/or deformable materials, the circuit 400 of FIG. 8 can stretch as a fluid is introduced to the internal cavity. In other words, the circuit 400 of FIG. 4 can be selectively inflated and deflated as the fluid is introduced and/or removed from the internal cavity. As the circuit 400 is inflated and deflated, one or more dimensions (e.g., length, cross-sectional area, etc.) of the traces 404ₐ will change. According to some non-limiting aspects, one or more dimensions of the electrical features (e.g., traces 404ₐ, 404_{b}) of the layups 402ₐ, 402_{b} can change between a fraction of a percent and about thirty percent as the circuit 400 transitions between an inflated and deflated condition. As the dimensions of the electrical features (e.g., traces 404ₐ, 404_{b}) change, one or more electrical parameters (e.g., an inductance, a resistance, a voltage drop, a capacitance, and an electromagnetic field, etc.) generated by the applied current as it traverses the circuit 400 will subsequently change. In other words, the electrical parameters of the circuit 400 will change as the circuit 400 is inflated and deflated, even though the applied current and/or voltage remains constant.

Although the deformation of the traces 404ₐ, 404_{b} discussed above a response to an internal pressure applied by the fluid onto the inner surfaces 413ₐ, 413_{b} of the layups 402ₐ, 402_{b}, it shall be appreciated that additional deformations may result from external pressures applied on the outer surfaces 415ₐ, 415_{b} of the layups 402ₐ, 402_{b}, which would cause further deformation of the layups 402ₐ, 402_{b} and traces 404ₐ, 404_{b}. According to some non-limiting aspects, a baseline deformation and a resulting electrical parameter can be established for an inflated condition of the circuit 400. Accordingly, any additional deformations and/or further differences in the generated electrical parameter due to those deformations can be compared to the baseline and utilized to characterize subsequent structural parameters associated with the circuit 400 associated with applied external pressures. This can be useful to monitor and characterize an environment in which the circuit 400 and/or the use of the circuit as an airbag, bladder, and/or cushion.

For example, according to the non-limiting aspect of FIG. 8, the coiled trace 404ₐ may generate a first inductive response when the circuit 400 is deflated and a second inductive response when the circuit 400 is inflated. The electrical parameter (e.g., strength of the inductive response) is a function of the distance between the coiled trace 404ₐ and the conductive layer 418. The conductive layer 418 can detect electrical parameters (e.g., inductance) generated by the coiled trace 404ₐ and can send signals associated with detected electrical parameters to one or more electronic components via one or more connecting traces 404ₐ, 404_{b} and/or one or more vias 406ₐ, 406_{b}. For example, the electronic components can include a microprocessor configured to determine a difference between detected electrical parameters and correlate the determined difference to a structural parameter (e.g., a strain, a stress, a pressure, a dimension, etc.) associated with the fluid-fillable circuit 400. In other words, the fluid-fillable circuit 400 can be configured such that an electrical characterization of the fluid-fillable circuit 400 can be used to characterize a physical condition of the fluid-fillable circuit 400. This can be useful in applications where the fluid-fillable circuit 400 is a bladder, or airbag of sorts, and the user wants to monitor and adjust the inflation for use as an adjustable cushion.

According to the non-limiting aspect of FIG. 8, the coiled trace 404ₐ and conductive layer 418 of FIG. 4 can be implemented to determine, quantify and/or otherwise characterize a strain applied to the fluid-fillable circuit 400 by the fluid in the internal cavity. However, it shall be appreciated that the coiled configuration of the trace 404ₐ of FIG. 8 is merely presented for illustrative purposes. According to other non-limiting aspects, other sensors and/or sensing techniques can be implemented in lieu of either the coiled trace 404ₐ and/or the conductive layer 418 of FIG. 4. For example, radio-frequency identification (“RFID”) techniques, surface-acoustic-wave sensors (“SAW”), and/or hall-effect sensors can be implemented to achieve a similar result. According to other non-limiting aspects, the electrical parameter can include an electrical resistance generated by the current applied to the circuit 400. As the circuit 400 is inflated and deflated, a cross-sectional area and length of the traces 404ₐ, 404_{b} changes, resulting in a difference in electrical resistance through the circuit 400. This difference can be similarly correlated to a structural parameter (e.g., a strain, a stress, a pressure, a dimension, etc.) associated with the fluid-fillable circuit 400.

It shall be further appreciated that different geometries will be better suited for different applications, and that some geometries may be easier to manufacture. For example, the rectangular form of the coil defined by the trace 404ₐ on the first layup 402ₐ may be easier to manufacture than a curved-spiral trace 404ₐ and more conducive for lamination. Additionally, the relative dimensions of the coil formed by the trace 404ₐ and the conductive layer 418 can be selected in accordance with user preference and/or intended application. In some non-limiting aspects, the coiled trace 404ₐ can have a width and length no more than 20 to 30 millimeters, making the circuit 400 ideal for a seamless integration into a higher-level of assembly. Furthermore, the number of coils formed by the trace 404ₐ can be configured for a desired performance and can account for any constraints in any direction, such as a maximum depth of coil descent, which may relate to a total number of coil turns the laminate structure may accommodate. For example, the one-and-a-half coils formed by the trace 404ₐ of FIG. 8 was shown to generate an effective 75% gain in inductance for the same area as a single coil.

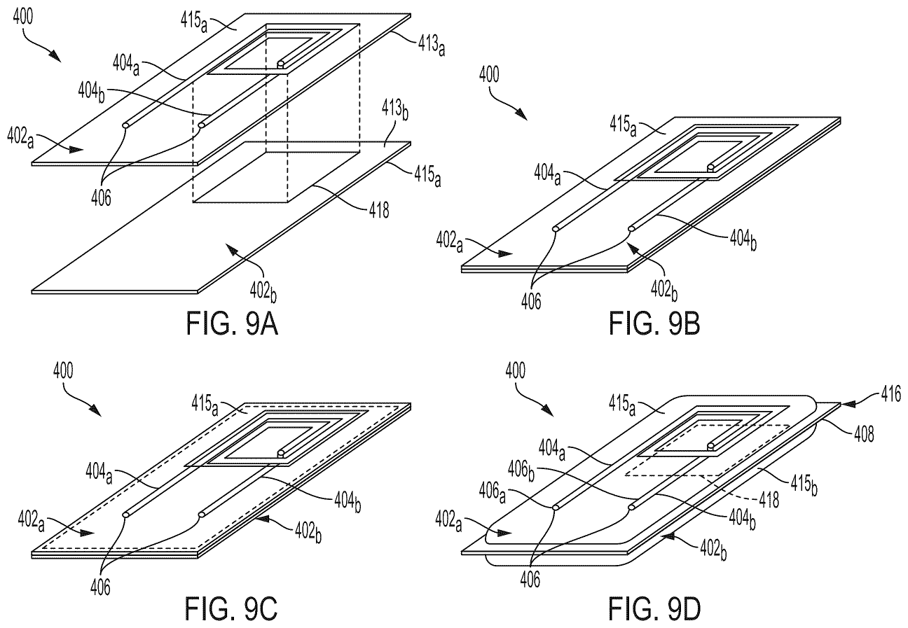

Referring now to FIGS. 9A-9D, an assembly of the fluid-fillable circuit 400 of FIG. 8 is depicted in accordance with at least one non-limiting aspect of the present disclosure. Specifically, FIG. 9A illustrates how the first layup 402ₐ can be aligned with the second layup 402_{b} such that the coiled trace 404ₐ aligns with the conductive layer 418. Moreover, the surface area of the conductive layer 418 is dimensioned such that the conductive layer 418 completely encompasses the coiled trace 404ₐ when the layups 402ₐ, 402_{b} are properly aligned.

In reference to FIG. 9B, a proper alignment of the first and second layups 402ₐ, 402_{b}—and more specifically, the coiled trace 404ₐ and conductive layer 418 is illustrated. After alignment, any corresponding electrical features on the first and second layups 402ₐ, 402_{b} can be electrically coupled. For example, an electronic component (or another fluid-fillable or non-fluid-fillable circuit containing an electronic component) may be coupled to one or more vias and/or contact points (as shown in FIG. 8) such that it is properly integrated with the circuit 400. Notably, according to the non-limiting aspect of FIG. 9, the coiled trace 404ₐ and conductive layer 418 are configured for inductive (e.g., electromagnetic) communication and thus, no electrical connection between the first and second layups 402ₐ, 402_{b} is necessary. The alignment of FIG. 9B prepares the layups 402ₐ, 402_{b} for the bonding procedure that will result in the edge joint 416.

Accordingly, the layups 402ₐ, 402_{b} of FIG. 9B are properly aligned and prepared for the bonding process, as depicted in FIG. 9C. A seal 908 can be formed between an inner surface 413ₐ of the first layup 402ₐ and an inner surface 413_{b} of the second layup 402_{b} by any known process of attachment (e.g., welding, soldering, fusing, stitching, adhesives, etc.). The seal 408 creates an edge joint 416 between the first and second layup 402ₐ, 402_{b}. In further reference of FIG. 9C, after the edge joint 416 is formed, a substantial portion of the inner surface 413ₐ of the first layup 402ₐ can face—but is not adhered to—a substantial portion of the inner surface 413_{b} of the second layup 402_{b}. Accordingly, the seal 408, which can be formed on every side of the circuit 400, can form an internal cavity (e.g., similar to the internal cavity 110 of FIG. 2) of the fluid-fillable circuit 400. A fluid can be inserted into and contained by the internal cavity formed by the inner surfaces 413ₐ, 413_{b}, the edge joint 416, and the seal 408. As more fluid is inserted within the internal cavity, the more pressure will be exerted on the inner surfaces 413ₐ, 413_{b} of the first and second layups 402ₐ, 402_{b}. Due to the deformable nature of the layups 402ₐ, 402_{b} and traces 404ₐ, 404_{b}, the circuit 400 can be inflated, as illustrated in FIG. 9D.

It shall be appreciated that while the foregoing embodiment has been described for a structure comprising two layups, any of the foregoing structures comprising a single layup and the associated description and methods of manufacture may be similarly applied to form a system similar to the system of FIGS. 8, 9A-D. Additional examples of inflatable circuits, correlation between an electrical parameter and a structural parameter of the fluid-fillable circuits, and method of manufacturing/using the fluid-fillable circuits are described in U.S. Provisional Application No. 63/272,487, titled DEVICES, SYSTEMS, AND METHODS FOR MAKING AND USING A FLUID-FILLABLE CIRCUIT, filed Oct. 27, 2021, the disclosure of which is hereby incorporated by reference in its entirety.

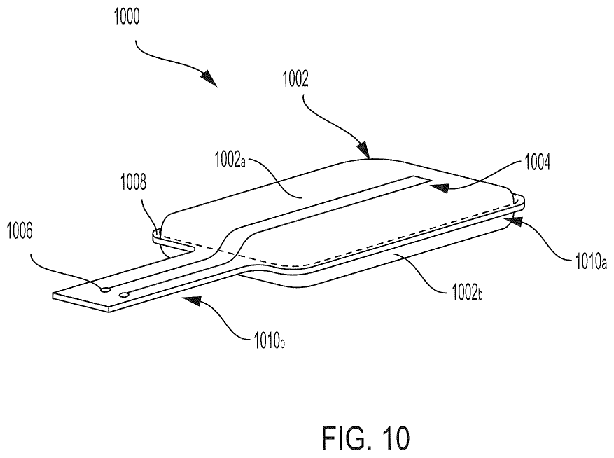

Referring now to FIG. 10, a partially fluid-fillable circuit 1000 is depicted in accordance with at least one non-limiting aspect of the present disclosure. The partially fluid-fillable circuit 1000 of FIG. 10 can include a layup 1002 comprising a first portion 1002ₐ and a second portion 1002_{b} and can be similarly constructed to the layups of the circuits discussed in reference to FIGS. 1, 4, 6, and 8. For example, the layup 1002 of FIG. 10 can include a multi-layer construction similar to those described in U.S. patent application Ser. No. 16/548,379 titled STRUCTURES WITH DEFORMABLE CONDUCTORS, which was filed on Aug. 22, 2019 and granted as U.S. Pat. No. 11,088,063 on Aug. 10, 2021, the disclosure of which is hereby incorporated by reference in its entirety. Alternately and/or additionally, the layup 1002 of FIG. 10 can include a two-layer construction. The layup 1002 of FIG. 10 can be constructed from flexible structures, such as those described in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety, can be used to fabricate one or more layers of the layup 1002. For example, the layup 1002 can be produced from one or more layers including a B-stage resin film, a C-stage resin film, an adhesive, a TPU, and/or a silicone material, amongst others.

However, unlike the circuits of FIGS. 1, 4, 6, and 8, the first portion of the layup 1002ₐ, and the second portion of the layup 1002_{b} of FIG. 10 are arranged and mechanically coupled to one another such that the partially fluid-fillable circuit 1000 defines a fluid-fillable portion 1010ₐ and a non-fluid-fillable portion 1010_{b}. According to the non-limiting aspect of FIG. 10, a perimeter of the first portion of the layup 1002ₐ can be mechanically coupled, fused, and/or otherwise integrated to a perimeter of the second portion of the layup 1002_{b} at a mechanical interface, creating a flanged seal 1008.

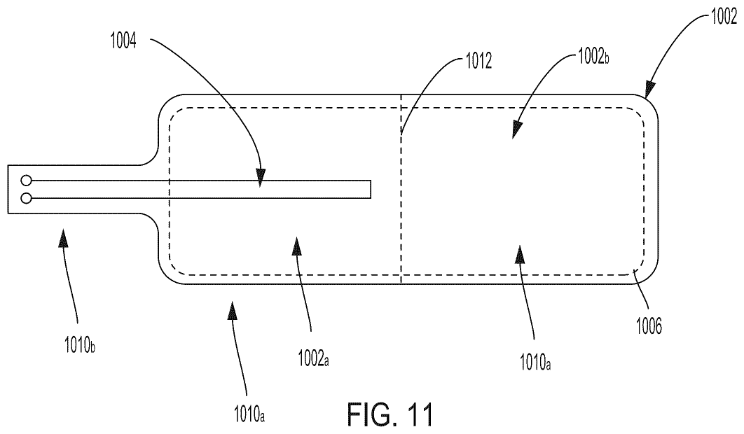

In further reference to FIG. 10, the flanged seal 1008 can be configured to define a fluid-fillable cavity of the fluid-fillable portion 1010ₐ of the partially fluid-fillable circuit 1000. The cavity can be filled with a fluid and thus, enables the fluid-fillable portion 1010ₐ to be inflated, as previously described in reference to the circuits of FIGS. 1, 4, 6, and 8. However, as will be described in further detail related to FIG. 11, only the first portion of the layup 1002ₐ extends beyond the seal 1008 external the cavity and thus, the non-fluid-fillable portion 1010_{b} of the circuit 1000 cannot be filled with a fluid. Accordingly, the circuit 1000 of FIG. 10 includes a fluid-fillable portion 1010ₐ and a non-fluid-fillable portion 1010_{b} and is therefore referred to as “partially fluid-fillable.”

Although the seal 1008 of FIG. 10 traverses a substantially rectangular perimeter of the fluid-fillable portion 1010ₐ of FIG. 10 and thus, defines a cavity of substantially rectangular shape, the flanged seal 1008 and the first portion of the layup 1002ₐ can be alternately configured such that the cavity defines any desired volume and/or geometry. Accordingly, it shall be appreciated that, according to other non-limiting aspects, the fluid-fillable portion 1010ₐ can define a cavity of any number of volumes and/or shapes, according to user preference and/or intended application.

Still referring to FIG. 10, the first portion of the layup 1002ₐ and/or the second portion of the layup 1002_{b} can include one or more traces 1004 that traverse the fluid-fillable portion 1010ₐ and/or the non-fluid-fillable portion 1010_{b} of the partially fluid-fillable circuit 1000. The one or more traces 1004 of the partially fluid-fillable circuit 1000 can be formed from a deformable conductor and thus, shall include but shall not be limited to, any conductor that is in a fluid state, or any conductor that can transition into a fluid state when an external stimulus (e.g. a strain, etc.) is applied to either the conductor or a structure surrounding the conductor, wherein the conductor is also capable of returning to a pre-stimulus state. According to the non-limiting aspect of FIGS. 10 and 11, the traces 1004 of the partially fluid-fillable circuit 1000 can be configured as a simple strain sensor. However, according to other non-limiting aspects, the traces 1004 of the partially fluid-fillable circuit 1000 can be alternately and/or additionally configured as a pressure and/or proximity sensor, a bus circuit for transporting electrical power to various components, and/or an antenna circuit for transmissions, amongst other circuit types.

Alternatively and/or additionally, the one or more traces 1004 can flow within channels defined within layers of the layup 1002 construction and therefore, undergo a fluid-type strain and/or shear within the trace 1004. The traces 1004 can be formed from magnesium or a magnesium-based compound, for example, which can result in a shear thinning material that flows readily when it undergoes a shearing-type stress. Thus, when a substrate layer of the layup 1002 is relaxed and returns to pre-strained state, a magnesium trace 1004 will return to a static, or sedimentary, viscosity. In other words, the one or more traces 1004 shall flow as substrate layers of the layup 1002 are deformed and thus, will not preclude the partially fluid-fillable circuits disclosed herein from undergoing the requisite transition between a first (e.g., deflated) state to a second (e.g., inflated) state while preserving electrical communication throughout the circuit in either the first or second state. In this regard, the one or more traces 1004 can include a deformable conductor that is distinguished from conventional conductors, such as copper wires, which would prevent the transition between the first and second states, or whose rigidity might break the electrical communication throughout the circuit during said transitions. According to other non-limiting aspects, one or more layers (e.g., a substrate layer) of the layup 1002 construction can include viscoelastic properties such that the layup 1002 can absorb vibrations. Such layers of the layup can be formed from a viscoelastic film, similar to those used in a 3M™ VHB™ viscoelastic tape, for example.

For example, according to some non-limiting aspects, the one or more traces 1004 of the circuit 1000 of FIG. 10 can include a deformable, conductive material, such as those disclosed in International Patent Application No. PCT/US2017/019762 titled LIQUID WIRE, which was filed on Feb. 27, 2017 and published on Sep. 8, 2017 as International Patent Publication No. WO2017/151523A1, the disclosure of which is hereby incorporated by reference in its entirety. The one or more traces 1004 can include a variety of forms, such as a liquid, a paste, a gel, and/or a powder, amongst others that would enable the one or more traces 1004 to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as a deformable conductors produced from a conductive gel (e.g., a gallium magnesium alloy, a gallium indium alloy, and/or other gallium-based alloys, etc.). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring one or more traces 1004 of varying forms and/or compositions to achieve the benefits disclosed herein.

According to the non-limiting aspect of FIG. 10, the first portion of the layup 1002ₐ can be positioned relative to the second portion of the layup 1002_{b} such that certain features of the first portion of the layup 1002ₐ align with corresponding features of the second portion of the layup 1002_{b}. For example, similar to the circuit 100 of FIG. 1, the one or more traces 1004 of the first portion of the layup 1002ₐ can terminate in one or more electrical vias 1006, which can be aligned with one or more electrical vias (not shown) that terminate one or more traces (not shown) of the second portion of the layup 1002_{b}. Once again, according to some non-limiting aspects, it may be preferable to fill the vias 1006 with a deformable conductor configured to convey electrical energy between corresponding traces 1004. The deformable conductor placed in the vias 1006 can be the same as, similar to, or different than the deformable conductors used for the traces 1004. However, according to other non-limiting aspects, the traces 1004 can be terminated in other forms of electrical contacts and/or interconnects, in accordance with user preference and/or intended application.

Referring now to FIG. 11, an assembly of the partially fluid-fillable circuit 1000 of FIG. 10 is depicted in accordance with at least one non-limiting aspect of the present disclosure. Specifically, FIG. 11 depicts a top view of the layup 1002 of the partially fluid-fillable circuit 1000 of FIG. 10 laid flat prior to assembly. As depicted in FIG. 11, the first portion of the layup 1002ₐ and the second portion of the layup 1002_{b} are positioned about by a fold 1012 on which the layup 1002 can be folded. After having folded the layup 1002 about the fold 1012, the first portion of the layup 1002ₐ and the second portion of the layup 1002_{b} are geometrically aligned and can be sealed together about the flanged seal 1008. The seal 1008 can be formed via a process configured to attach the outer perimeters of the layup 1002, including any known process that uses heat, pressure, radio-frequency energy, and/or additional materials for attachment (e.g., welding, soldering, fusing, stitching, adhesives, etc.). Accordingly, the folded fold 1012 and the flanged seal 1008 collectively define the cavity of the fluid-fillable portion 1010ₐ of the partially fluid-fillable circuit 1000. As depicted in FIG. 11, once the first portion of the layup 1002ₐ and the second portion of the layup 1002_{b} are sealed, a part of the first portion of the layup 1002ₐ with the traces 1004 extends beyond the seal 1008, thereby forming the non-fluid fillable portion 1010ₐ of the partially fluid-fillable circuit 1000. Alternatively, the non-fluid-fillable portion 1010_{b} can be formed from a second layup attached to the fluid-fillable portion 1010ₐ, and vias may be used to establish electrical communication between traces of the non-fluid-fillable portion 1010_{b} and the fluid-fillable portion 1010ₐ. For example, vias can be positioned at flange 1008 or somewhere in the fluid-fillable (e.g., non-unitized) region.

Once assembled, the fluid-fillable portion 1010ₐ of the circuit 1000 can be inflated using various known methods of inflation, such as the method described in U.S. patent application Ser. No. 11/107,354 titled FLUID-FILLED BLADDER FOR FOOTWEAR AND OTHER APPLICATIONS, which was filed on Apr. 14, 2004 and granted on Jul. 22, 2009 as U.S. Pat. No. 7,401,369, the disclosure of which is hereby incorporated by reference in its entirety. For example, the layup 1002 can include at least a portion of the layup 1002 can be fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 1000 of FIG. 10, and fluid can be selectively introduced and/or removed from the internal cavity via the valve assembly.

It shall be appreciated that the assembly illustrated by FIG. 11 is only one way to assemble the partially fluid-fillable circuit 1000 of FIG. 10. According to other non-limiting aspects, the first portion of the layup 1002ₐ and the second portion of the layup 1002_{b} can include substantially similar and/or overlapping geometries in a region intended to form the non-fluid-fillable portion 1010_{b}. For example, the layup can be symmetrically configured about the fold line 1012 such that the first portion of the layup 1002ₐ has the same geometry as the second portion of the layup 1002_{b} and, when unitized about fold 1012, the first portion of the layup 1002ₐ and the second portion of the layup 1002_{b} can be unitized to form the non-fluid-fillable portion 1010_{b} of the partially fluid-fillable circuit 1000. As used herein, the term “unitize” shall include any means of mechanically coupling corresponding surfaces of any two layups, such that the unitized surfaces of those layups are mechanically inseparable in a particular region of a partially fluid-fillable circuit. In other words, surfaces of layups that are unitized cannot separate and thus, cannot define a cavity in the non-fluid-fillable portion of the partially fluid-fillable circuit. Unitization, for example, can be accomplished via the application of pressure, adhesives, and/or heat, such that two layups are mechanically inseparable in a particular region of a partially fluid-fillable circuit. However, the present disclosure contemplates other methods of unitizing two layups. Accordingly, when assembling the partially fluid-fillable circuit 1000 of FIG. 10, an inner surface of the first portion of the layup 1002ₐ can be unitized to an inner surface of the second portion of the layup 1002_{b} at the non-fluid-fillable portion 1010_{b} of the partially fluid-fillable circuit 1000, such that the first portion of the layup 1002ₐ is integral with the second portion of the layup 1002_{b} at the non-fluid-fillable portion 1010_{b} of the partially fluid-fillable circuit 1000. However, according to this non-limiting aspect, the first portion of the layup 1002ₐ remains mechanically separate from the second portion of the layup 1002_{b} at the fluid-fillable portion 1010_{b}, such that the flanged seal 1008, fold 1012, and a boundary of the unitized portion of the first portion of the layup 1002ₐ and the second portion of the layup 1002_{b} define a fluid-fillable cavity of the fluid-fillable portion 1010ₐ of the partially fluid-fillable circuit 1000. Additionally and/or alternately, the non-fluid-fillable portion 1010_{b} can also include two or more stacks overlaid and unitized to one another.

A partially fluid-fillable circuit, such as the partially fluid-fillable circuit 1000 of FIGS. 10 and 11, can be beneficial because the fluid-fillable portion 1010ₐ can be positioned and/or integrated into a housing at a desired location for particular functions (e.g., sensing at a particular location of interest, relative movement of an electronic component positioned at a particular location of interest, etc.), while maintaining electrical continuity with the non-fluid-fillable portion 1010_{b}. The non-fluid-fillable portion 1010_{b} can be positioned and/or integrated into a housing at another desired location, for example a location of the housing where the benefits of an inflated or selectively inflatable bladder are not required, but where it may be desirable to route and conduct signals or electrical currents, and/or provide other types of sensing structures formed from traces made from the deformable conductor. This can be beneficial for integrating the partially fluid-fillable circuit 1000 into wearable articles and promoting comfort without compromising the utility of the partially fluid-fillable circuit 1000.

Additionally and/or alternately, it may be desirable to couple certain electronic components to the fluid-fillable portion 1010ₐ and/or the non-fluid-fillable portion 1010_{b}. For example, according to the non-limiting aspect of FIGS. 10 and 11, the traces 1004 can be configured as a simple sensor, such that the traces 1004 generate varying electrical parameters (e.g., an inductance, a resistance, a voltage drop, a capacitance, signal, an electromagnetic field, etc.) as an electrical current and/or potential is be applied to the circuit 1000 and the fluid-fillable portion 1010ₐ is inflated and/or deflated or the non-fluid-fillable portion 1010_{b} is stretched and/or otherwise deformed. In some examples, it might be beneficial to couple one or more electronic components (e.g., a power source, a microprocessor, a logic-based controller, a transceiver, an electrode, a LED bank, a pump etc.) which may be mechanically secured to and/or otherwise integrated into the first portion of the layup 1002ₐ at the non-fluid-fillable portion 1010_{b} of the partially fluid-fillable circuit 1000.

For example, a microprocessor, on its own or hosted as part of an auxiliary component such as on a flex printed circuit board (“PCB”), can be coupled to the non-fluid-fillable portion 1010_{b} and configured to correlate the varying electrical parameters (e.g., an inductance, a resistance, a voltage drop, a capacitance, signal, an electromagnetic field, etc.) to a structural parameter (e.g., a strain, a stress, a pressure, a dimension, etc.). Alternately and/or additionally, either a receiver, a transmitter, or a transceiver can be coupled to the non-fluid-fillable portion 1010_{b} and configured to transmit and/or receive signals to and/or from the partially fluid-fillable circuit 1000 and/or its various electronic components. Alternately and/or additionally, an LED array can be coupled to the non-fluid-fillable portion 1010_{b} and configured to illuminate one or more LEDs based on electrical parameters (e.g., an inductance, a resistance, a voltage drop, a capacitance, signal, an electromagnetic field, etc.) generated by the traces 1004 and/or structural parameters (e.g., a strain, a stress, a pressure, a dimension, etc.) determined by a microprocessor. Accordingly, various electronic components can be coupled to non-fluid-fillable portion 1010_{b} and configured to monitor, transmit, and/or otherwise dynamically or statically calculate, infer or determine one or more physical or structural characteristics or conditions of the partially fluid-fillable circuit 1000, and/or a stimuli applied to the partially fluid-fillable circuit 1000.

It shall be appreciated that, according to non-limiting aspects wherein the partially fluid-fillable circuit 1000 is integrated into a wearable article, comfort can be promoted by mounting various electronic components to the non-fluid-fillable portion 1010_{b}, as the electronic components will not be pressed against the user's body, joint, or appendage as the fluid-fillable portion 1010ₐ is selectively inflated and deflated. However, according to other non-limiting aspects, it may be beneficial to coupled certain electronic components (e.g., electrodes) to the fluid-fillable portion 1010ₐ of the partially fluid-fillable circuit 1000. For example, according to some non-limiting aspects, wherein the partially fluid-fillable circuit 1000 is integrated into a wearable article, an electrode can be coupled to the fluid-fillable portion 1010ₐ of the partially fluid-fillable circuit 1000 and configured to monitor and/or stimulate a user's body part at a particular location. Accordingly, as the fluid-fillable portion 1010ₐ of the partially fluid-fillable circuit 1000 is inflated and/or deflated, the electrode can be biased against the user, according to user preference and/or intended application.

In some non-limiting aspects, the microprocessor can be used to monitor the signal generated by an electrode (e.g., an EMG) to assist in determining the correct amount of biasing required from the inflatable portion. A feedback system can be provided (e.g., a system with a processor or microprocessor) electrically coupled to one or more electrodes, and a inflation pump electrically coupled with the processor, which may configured to inflate or deflate the fluid fillable portion 1010ₐ of the partially-fluid-fillable circuit 1000 to optimize the signal received from the electrode, in response to commands received from the processor. Sensors integrated to the fluid-fillable portion 1010ₐ may transmit signals to the processor, which may actively monitor the pressure within the partially-fluid-fillable portion 1010ₐ. As such, the feedback system can prevent the fluid-fillable portion 1010ₐ from becoming filled beyond a predetermined threshold (e.g., over-inflation, under-inflation, etc.) or can set an inflation range or threshold value specified by a user that corresponds to an acceptable level of comfort.

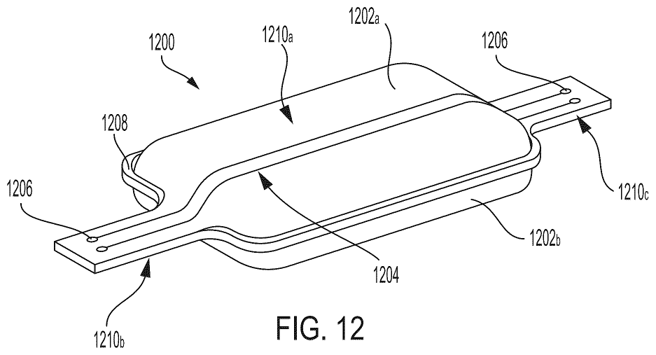

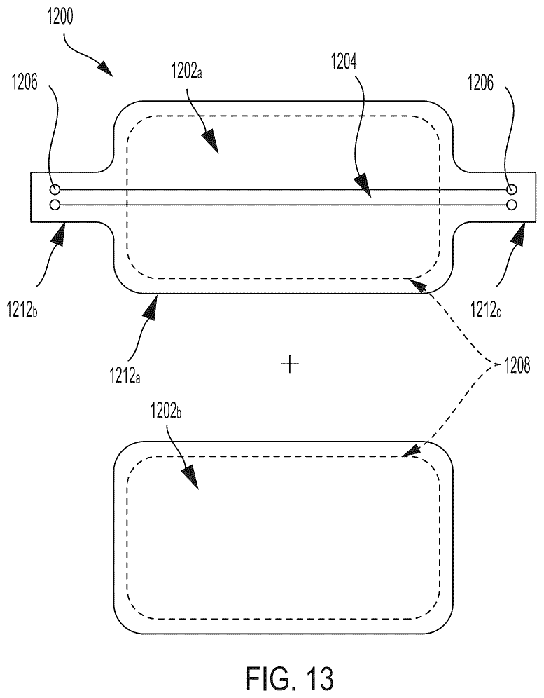

Referring now to FIG. 12, another partially fluid-fillable circuit 1200 is depicted in accordance with at least one non-limiting aspect of the present disclosure. According to the non-limiting aspect of FIG. 12, the partially fluid-fillable circuit 1200 can include at least a first layup 1202ₐ mechanically coupled to a second layup 1202_{b}, e.g., at a seal 1208, as will be discussed in further detail in reference to FIG. 13. The first layup 1202ₐ can include one or more traces 1204 formed from a deformable conductor, which traverse the fluid-fillable portion 1210ₐ, the first non-fluid-fillable portion 1210_{b}, and the second non-fluid-fillable portion 1210_{c} of the partially fluid-fillable circuit 1200. Alternatively, one of the fluid-fillable portion 1210ₐ and the non-fluid-fillable portion 1210_{b} may be provided on one of the first layup 1202ₐ and the second layup 1202_{b}, and the other of the fluid-fillable portion 1210ₐ and the non-fluid-fillable portion 1210_{b} may be provided on the other of the first layup 1202ₐ and the second layup 1202_{b}. The one or more traces 1204 can terminate in one or more electrical vias 1206—or other electrical interconnects—positioned on the first non-fluid-fillable portion 1210_{b}, and the second non-fluid-fillable portion 1210_{c} of the partially fluid-fillable circuit 1200. The first layup 1202ₐ, and second layup 1202_{b} of FIG. 12 can be configured similar to the layup 1002 of FIG. 10. Likewise, the deformable conductor of the traces 1204 and electrical vias 1206 of FIG. 12 can be configured similar to the deformable conductor of the traces 1004 and electrical vias 1006 of FIG. 10, respectively. It shall be further appreciated that either and/or both of the non-inflatable portions can be mechanically and electrically coupled to the inflatable portion and not formed integrally with either of the first and second layups that form the inflatable portion as well.

In further reference to FIG. 12, the flanged seal 1208 is configured to define a fluid-fillable cavity of a fluid-fillable portion 1210ₐ of the partially fluid-fillable circuit 1200. The cavity can be filled with a fluid and thus, enables the fluid-fillable portion 1210ₐ to be inflated, similar to the partially fillable circuit 1000 of FIG. 10 and as previously described in reference to the circuits of FIGS. 1, 4, 6, and 8. However, as will be described in further detail related to FIG. 13, a first and second portion 1212_{b}, 1212_{c} of the first layup 1202ₐ traverses beyond the seal 1208 external to the cavity and thus, the non-fluid-fillable portions 1210_{b}, 1210_{c} of the circuit 1200 cannot be filled with a fluid. Accordingly, the circuit 1200 of FIG. 12 includes a fluid-fillable portion 1210ₐ and two non-fluid-fillable portions 1210_{b}, 1210_{c} and thus, is partially fluid-fillable.

Although the seal 1208 of FIG. 12 traverses a substantially rectangular perimeter of the fluid-fillable portion 1210ₐ of FIG. 12, thereby defining a cavity of substantially rectangular shape, the flanged seal 1208 and the first and second layups 1202ₐ, 1202_{b} can be alternately configured such that the cavity defines any desired volume and/or geometry. Accordingly, it shall be appreciated that, according to other non-limiting aspects, the fluid-fillable portion 1210ₐ can define a cavity of any number of volumes and/or shapes, according to user preference and/or intended application.

Referring now to FIG. 13, an assembly of the partially fluid-fillable circuit 1200 of FIG. 12 is depicted in accordance with at least one non-limiting aspect of the present disclosure. Specifically, FIG. 12 depicts a top view of the first layup 1202ₐ and the second layup 1202_{b} of the partially fluid-fillable circuit 1200 of FIG. 12 laid flat prior to assembly. According to the non-limiting aspect of FIG. 13, the first layup 1202ₐ can include a first portion 1212ₐ, a second portion 1212_{b}, and a third portion 1212_{c}, each of which can be configured to correspond to the fluid-fillable portion 1210ₐ or the non-fluid-fillable portion 1210_{b} of the partially fluid-fillable circuit 1200, as depicted in FIG. 12. According to the non-limiting aspect of FIG. 13, a first portion 1212ₐ of the first layup 1202ₐ can include a substantially similar geometry that corresponds to the entire geometry of the second layup 1202_{b}. As such, the first portion 1212ₐ of the first layup 1202ₐ can be positioned above and aligned with the second layup 1202_{b}. After having positioned the first layup 1202ₐ above the second layup 1202_{b}, the second layup 1202_{b} can be sealed to the first layup 1202ₐ about the seal 1208, which traverses a perimeter of the first portion 1212ₐ of the first layup 1202ₐ.

Once again, the seal 1208 can be formed via a process configured to attach an outer perimeters of the second layup 1202_{b} to the first portion 1212ₐ of the first layup 1202ₐ, including any known process that uses heat, pressure, radio-frequency energy, and/or additional materials for attachment (e.g., welding, soldering, fusing, stitching, adhesives, etc.). Accordingly, an inner surface of the first portion 1212ₐ of the first layup 1202ₐ, and inner surface of the second layup 1202_{b}, and the seal 1208 collectively define the cavity of the fluid-fillable portion 1210ₐ of the partially fluid-fillable circuit 1200. As depicted in FIG. 12, once the first portion 1212ₐ of the first layup 1202ₐ is mechanically coupled to the second layup 1202_{b} via seal 1208, the second portion 1212_{b} and the third portion 1212_{b} of the first layup 1202ₐ extend beyond the seal 1208, thereby forming the non-fluid fillable portions 1210_{b}, 1210_{c} of the partially fluid-fillable circuit 1200 of FIG. 12.

Upon assembly, the fluid-fillable portion 1210ₐ of the circuit 1200 of FIG. 12 can be inflated using several methods of inflation, including those discussed in reference to FIG. 11. For example, the first layup 1202ₐ and/or the second layup 1202_{b} can include at least a portion fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 1200 of FIG. 12, and fluid can be selectively introduced and/or removed from the internal cavity via the valve assembly. Accordingly, the partially fluid-fillable circuit 1200 of FIG. 12 can realize all of the benefits described in reference to the circuit 1000 of FIGS. 10 and 11, including a more precise integration into a housing, the particular placement of electronic components, and an overall enhanced comfort for a user when integrated into a wearable article.

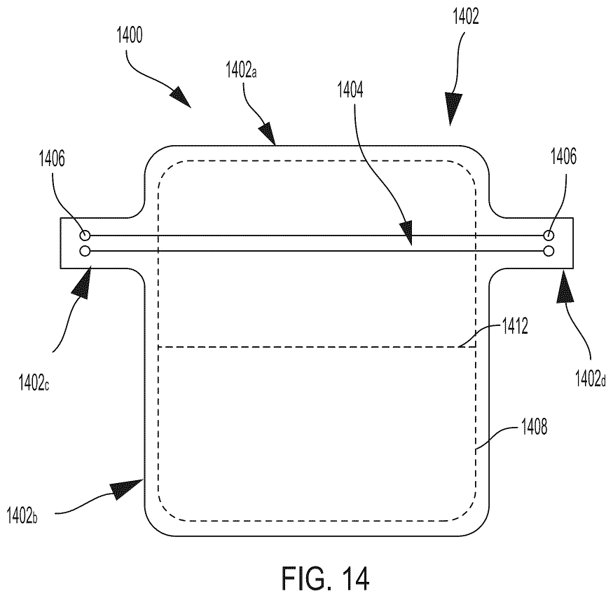

Referring now to FIG. 14, another assembly of another partially fluid-fillable circuit 1400 is depicted in accordance with at least one non-limiting aspect of the present disclosure. Specifically, FIG. 14 depicts a top view of a single layup 1402 configured with a fold 1412. According to the non-limiting aspect of FIG. 14, the partially fluid-fillable circuit 1400 can include a layup 1402 that includes a first portion 1402ₐ, a second portion 1402_{b}, a third portion 1402_{c}, and a fourth portion 1402_{d}. The first portion 1402ₐ and the second portion 1402_{b} of the layup 1402 have substantially similar geometries that correspond to one another and are disposed about the fold 1412. As will be described in further detail, the first and second portions 1402ₐ, 1402_{b} of the layup 1402 can be configured to define a fluid-fillable portion of the partially fluid-fillable circuit 1400. Once again, the layup 1402 can include one or more traces 1404 formed from a deformable conductor, which can traverse a fluid-fillable portion defined the first and second portions 1402ₐ, 1402_{b} of the layup 1402. The one or more traces 1404 can terminate in one or more electrical vias 1406—or other electrical interconnects—positioned on the third and fourth portions 1402_{c}, 1402_{d} of the layup 1402. The layup 1402 of FIG. 14 can be configured similar to the layup 1002 of FIG. 10. Likewise, the deformable conductor of the traces 1404 and electrical vias 1406 of FIG. 14 can be configured similar to the deformable conductor of the traces 1004 and electrical vias 1006 of FIG. 10, respectively. Alternately and/or additionally, a non-fluid-fillable portion of the partially fluid-fillable circuit 1400 can be formed from a second layup attached to the first and second portions 1402ₐ, 1402_{b} of the layup 1402 configured to define a fluid-fillable portion fluid-fillable portion. Once again, vias can be used to establish electrical communication between traces of the non-fluid-fillable portion and the first and second portions 1402ₐ, 1402_{b} of the layup 1402 configured to define a fluid-fillable portion fluid-fillable portion. For example, vias can be positioned at flange or somewhere in the fluid-fillable (e.g., non-unitized) region.

As depicted in FIG. 14, the first portion of the layup 1402ₐ and the second portion of the layup 1402_{b} are positioned about by a fold 1412 on which the layup 1402 can be folded. After having folded the layup 1402 about the fold 1412, the first portion of the layup 1402ₐ and the second portion of the layup 1402_{b} are geometrically aligned and can be sealed together about the flanged seal 1408. Once again, the seal 1408 can be formed via a process configured to attach the outer perimeters of the first and second portions 1402ₐ, 1402_{b} of the layup 1402, including any known process that uses heat, pressure, radio-frequency energy, and/or additional materials for attachment (e.g., welding, soldering, fusing, stitching, adhesives, etc.). Accordingly, the folded fold 1412 and the flanged seal 1408 can collectively define a cavity of a fluid-fillable portion of the partially fluid-fillable circuit 1400. As depicted in FIG. 14, once the first portion of the layup 1402ₐ and the second portion of the layup 1402_{b} are sealed, the third and fourth portions 1402_{c}, 1402_{d} of the layup 1402 with the traces 1404 can extend beyond the seal 1408, thereby forming a non-fluid fillable portion of the partially fluid-fillable circuit 1400.

Although the seal 1408 and the fold 1412 of FIG. 14 collectively traverse a substantially rectangular perimeter of the first and second portions 1402ₐ, 1402_{b} of the layup 1402 of FIG. 14, thereby defining a cavity of substantially rectangular shape, the seal 1408, fold 1412, and layup 1402 can be alternately configured such that the cavity defines any desired volume and/or geometry. Accordingly, it shall be appreciated that, according to other non-limiting aspects, the seal 1408, fold 1412, and layup 1402 can be alternately configured to define a fluid-fillable portion with a cavity of any number of volumes and/or shapes, according to user preference and/or intended application.

Once assembled, the partially fluid-fillable circuit 1400 can be substantially similar to the partially fluid-fillable circuit 1200 of FIG. 12, except the folded fold 1412 along with the flanged seal 1408 define the cavity, along with an inner surface of the first portion 1402ₐ and an inner surface of the second portion 1402_{b} of the layup 1402. Upon assembly, the fluid-fillable portion of the circuit 1400 of FIG. 14 can be inflated using several methods of inflation, including those discussed in reference to FIG. 11. For example, the first portion 1402ₐ of the layup 1400 and/or the second portion 1402_{b} of the layup 1402 can include a multi-layer construction and can be fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 1400 of FIG. 14, and fluid can be selectively introduced and/or removed from the internal cavity via the valve assembly. Accordingly, the partially fluid-fillable circuit 1400 of FIG. 14 can realize all of the benefits described in reference to the circuit 1000 of FIGS. 10 and 11, including a more precise integration into a housing, the particular placement of electronic components, and an overall enhanced comfort for a user when integrated into a wearable article.

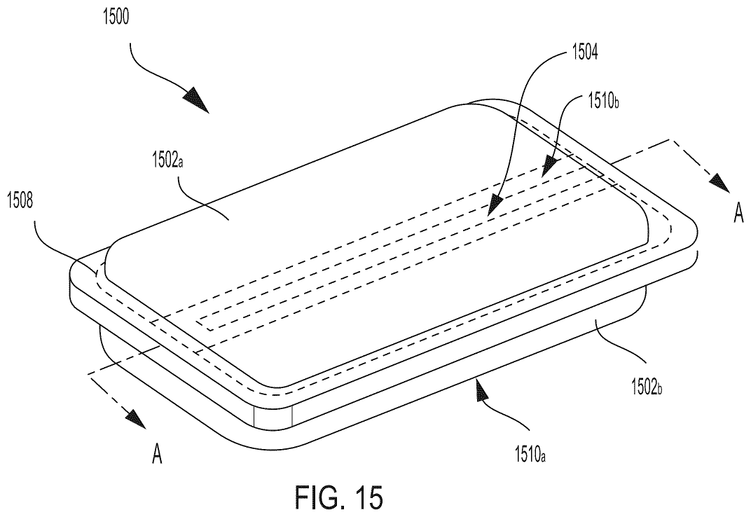

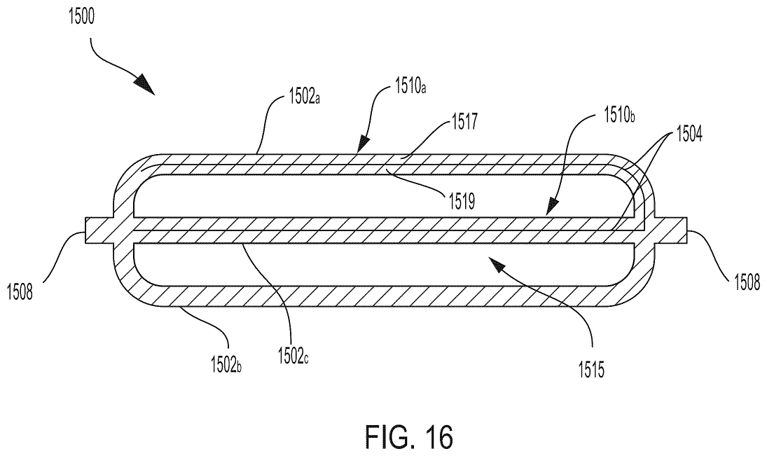

Referring now to FIG. 15, another partially fluid-fillable circuit 1500 is depicted in accordance with at least one non-limiting aspect of the present disclosure. According to the non-limiting aspect of FIG. 15, the partially fluid-fillable circuit 1500 can include a first layup 1502ₐ, mechanically coupled to a second layup 1502_{b} via a seal 1508, as previously discussed. Additionally, one or more traces 1504 formed from a deformable conductor can traverse an external surface of the first layup 1502ₐ and/or a non-fluid-fillable portion 1510_{b} of the partially fluid-fillable circuit 1500 that traverses through the cavity of the fluid-fillable portion 1510ₐ. The non-fluid-fillable portion 1510_{b} will be described in further detail in reference to FIG. 16. The first layup 1502ₐ, and second layup 1502_{b} of FIG. 15 can be configured similar to the layup 1002 of FIG. 10. Likewise, the deformable conductor of the traces 1504 of FIG. 15 can be configured similar to the deformable conductor of the traces 1004 of FIG. 10. Alternately, it shall be appreciated that, according to some non-limiting aspects, the partially fluid-fillable circuit 1500 of FIG. 15 can be formed from a single layup.

According to the non-limiting aspect of FIG. 15, the partially fluid-fillable circuit 1500 can include a contained geometry, since the non-fluid-fillable portion 1510 traverses through the cavity of the fluid-fillable portion 1510ₐ and is completely encompassed by the fluid-fillable portion 1510ₐ. Accordingly, any traces 1504 and/or electronic components (e.g., a power source, a microprocessor, a logic-based controller, a transceiver, an electrode, one or more LEDs, a pump etc.) of the non-fluid-fillable portion 1510_{b} can be completely encompassed by the fluid-fillable portion 1510ₐ of the partially fluid-fillable circuit 1500. These features can enable the partially fluid-fillable circuit 1500 to be particularly useful for a variety of end-uses including, without limitation, a midsole of a shoe, a personal massage device, protective padding, and/or a recreational ball, all of which are examples of end-uses that could benefit from the contained geometry and can be selectively inflated and/or deflated according to user preference and/or intended application.

Referring now to FIG. 16, a cross-sectioned side view of the partially fluid-fillable circuit 1500 of FIG. 15 is depicted in accordance with at least one non-limiting aspect of the present disclosure. Specifically, the cross-section of FIG. 16 is taken along line A-A, as depicted in FIG. 15. Accordingly, the first layup 1502ₐ and second layup 1502_{b} are mechanically coupled via the seal 1508, which can be formed via any known process that uses heat, pressure, radio-frequency energy, and/or additional materials for attachment (e.g., welding, soldering, fusing, stitching, adhesives, etc.), thereby defining a cavity 1515 between the inner surfaces of the first and second layups 1502ₐ, 1502_{b}. As such, the outer surfaces of the first and second layups 1502ₐ, 1502_{b} define a fluid-fillable portion 1510ₐ of the partially fluid-fillable circuit 1500.

The seal 1508 of FIGS. 15 and 16 traverses a substantially rectangular perimeter of the fluid-fillable portion 1510ₐ, thereby imbuing the cavity 1515 with a substantially rectangular shape and volume. However, the flanged seal 1508 and the first and second layups 1502ₐ, 1502_{b}can be alternately configured such that the cavity 1515 defines any desired volume and/or geometry. Accordingly, it shall be appreciated that, according to other non-limiting aspects, the fluid-fillable portion 1510ₐ can define a cavity 1515 of any number of volumes and/or shapes, according to user preference and/or intended application.

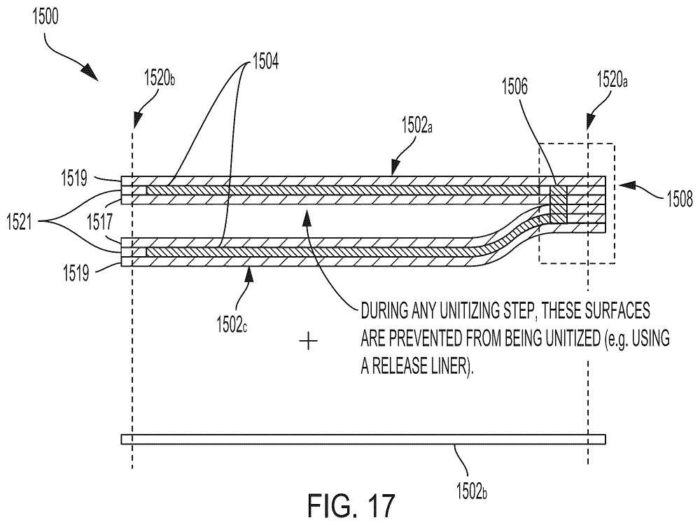

Notably, the partially fluid-fillable circuit 1500 of FIGS. 15 and 16 can include a non-fluid-fillable portion 1510_{b} that traverses through the cavity 1515 defined by and intermediate to the first and second layups 1502ₐ, 1502_{b}. As previously discussed, the one or more traces 1504 formed from a deformable can traverse the first layup 1502ₐ that, as will be described in further detail in reference to FIG. 17, can be unitized at the seal 1508 to a third layup 1502_{c} that defines the non-fluid-fillable portion 1510_{b}, which traverses the cavity through 1515. According to the non-limiting aspect of FIG. 16, the first layup 1502ₐ and the third layup 1502_{c} can include, at least, a substrate layer 1517 and an encapsulation layer 1519, as previously described. The deformable conductor that defines the traces 1504 can be deposited directly on the substrate layer 1517 and encapsulated by the encapsulation layer 1519. However, according to other non-limiting aspects, the first layup 1502ₐ can further include a stencil layer that defines a channel in which the deformable conductor can be deposited, thereby forming the traces 1504.

Upon assembly, the fluid-fillable portion 1510ₐ of the circuit 1500 can be inflated using several methods of inflation, including those discussed in reference to FIG. 11. For example, the first layup 1502ₐ and/or the second layup 1502_{b} can include at least a portion fabricated from a microlayer membrane that can accommodate a needle, an inflation nozzle, an inflation electrode, or another inflation device during inflation but can be sealed via the application of radio frequency energy or heat once a desired degree of inflation is achieved. However, according to other non-limiting aspects, a valve assembly can be mechanically coupled to the circuit 1500 of FIG. 16, and fluid can be selectively introduced and/or removed from the internal cavity 1515 via the valve assembly. Accordingly, the partially fluid-fillable circuit 1500 of FIG. 16 can realize all of the benefits described in reference to the circuit 1000 of FIGS. 10 and 11, including a more precise integration into a housing, the particular placement of electronic components, and an overall enhanced comfort for a user when integrated into a wearable article.

Referring now to FIG. 17, an assembly of the partially fluid-fillable circuit 1500 of FIG. 15 is depicted in accordance with at least one non-limiting aspect of the present disclosure. According to the non-limiting aspect of FIG. 17, the particular constructions of the first layup 1502ₐ, the second layup 1502_{b}, and the third layup 1502_{c} are depicted in further detail. For example, the substrate layer 1517 and encapsulation layer 1519 are depicted as encompassing the deformable conductor that defines the traces 1504 of the partially fluid-fillable circuit 1500. According to the non-limiting aspect of FIG. 17, the traces 1504 of the first layup 1502ₐ and the third layup 1502_{c} can be electrically coupled via one or more electrical vias 1506, which can be placed within the flanged seal 1508. Additionally, according to some non-limiting aspects, the first and third layups 1502ₐ, 1502_{c} can include a stencil layer 1521 that includes channels in which the deformable conductor can be deposited, thereby providing a more robust structural formation of the traces 1504. The traces 1504 in the first layup 1502ₐ that defines an outer wall of the cavity 1515 (FIG. 16) can be optional and, according to some non-limiting aspects, only the third layup 1502_{c} that defines the non-fluid-fillable portion 1510_{b} of the circuit 1500 can include traces 1504.

According to the non-limiting aspect of FIG. 17, the second layup 1502_{b} includes a single layer construction that does not contain any traces 1504 and thus, is simply configured to function as an outer wall of the cavity 1515 (FIG. 16). However, according to other non-limiting aspects, any of the first layup 1502ₐ, the second layup 1502_{b}, and third layup 1502_{c} can include traces 1504 formed from a deformable conductor and/or varying constructions involving various layers. Furthermore, although the traces 1504 of FIG. 17 are configured such that the circuit 1500 functions as a simple strain gauge, it shall be appreciated that, according to other non-limiting aspects, the traces 1504 of the partially fluid-fillable circuit 1500 can be alternately and/or additionally configured to function as a pressure and/or proximity sensor, a bus circuit for transporting electrical power to various components, and/or an antenna circuit for transmissions, amongst other circuit types.

In further reference to FIG. 17, bond lines 1520ₐ, 1520_{b} of the flanged seal 1508 is depicted about which the first layup 1502ₐ, the second layup 1502_{b}, the third layup 1502_{c} can be unitized. According to the non-limiting aspect of FIG. 17, the first layup 1502ₐ and third layup 1502_{c} are unitized about a first bond line 1520ₐ, thereby forming a portion of the flanged seal 1508. However, the second layup 1502_{b} has yet to be unitized to the first layup 1502ₐ and third layup 1502_{c} about the bond line 1520 of the flanged seal 1508. Once the second layup 1502_{b} is unitized to the first layup 1502ₐ and the third layup 1502_{c} about the first bond line 1520ₐ, the flanged seal 1508 will be complete and the cavity 1515 (FIG. 16) can be filled with a fluid.

Since the partially fluid-fillable circuit 1500 of FIG. 17 is assembled without folding, it can be beneficial if adjacent surfaces of the first layup 1502ₐ, the second layup 1502_{b}, and the third layup 1502_{c} are properly spaced during unitization. For example, according to non-limiting aspects where a plate heat press is being used in the unitizing process, a user should ensure that there is some separation and/or a release liner (e.g., a PTFE film) that prevents the layers from being unitized. Proper spacing can ensure proper mechanical separation and unitization in the desired locations. For example, unitization may only be desired at the flanged seal 1508. Accordingly, separation of adjacent surfaces of the first layup 1502ₐ, the second layup 1502_{b}, and the third layup 1502_{c} can be achieved via a release liner positioned between portions of the surfaces that are intended to be separated. After the layups 1502ₐ, 1502_{b}, 1502_{c} are unitized at the flanged seal 1508, the releasable liner can be removed such that adjacent surfaces of the first layup 1502ₐ, the second layup 1502_{b}, and the third layup 1502_{c} define a cavity 1515 (FIG. 16), as desired.

However, precise spacing may not be necessary. For example, according to some non-limiting aspects, adjacent surfaces of the first layup 1502ₐ, the second layup 1502_{b}, and the third layup 1502_{c} can be placed in contact and a unitizing tool can include features that only contact the areas to be unitized, reducing the need for precise spacing and increasing the manufacturing margin of error. Additional examples of inflatable circuits are described in U.S. Provisional Application No. 63/366,778, titled DEVICES, SYSTEMS, AND METHODS FOR MAKING AND USING A PARTIALLY FLUID-FILLABLE CIRCUIT, filed Jun. 22, 2022, the disclosure of which is hereby incorporated by reference in its entirety.

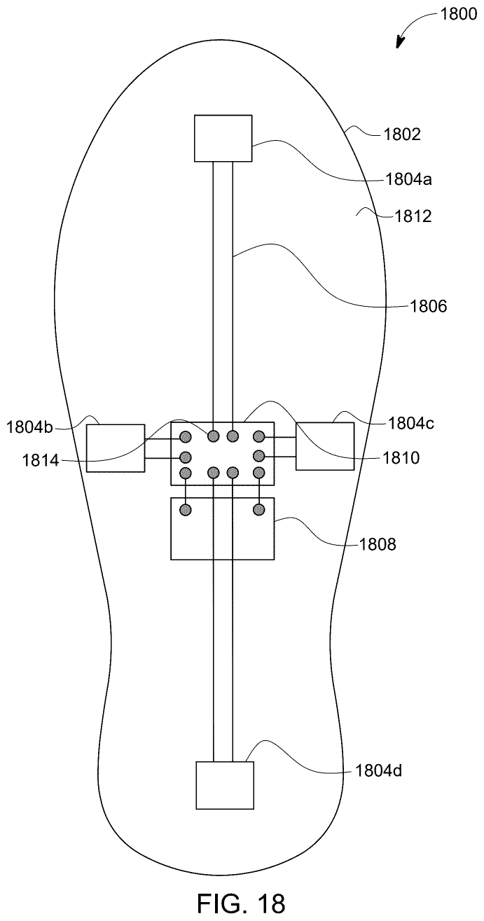

Referring now to FIG. 18, an article 1800 configured for navigating a virtual environment based on physical motions of a user is depicted in accordance with at least one non-limiting aspect of the present disclosure. As used herein, a user's motion or a physical motion may refer to any type of action or posture of the user, including, but not limited to, a position, an orientation, a speed, an acceleration, a weight transfer, a pose, and/or a prescribed sequence (e.g., walking, jumping, crouching). According to the non-limiting aspect of FIG. 18, the article 1800 can be configured as a foot base to be placed under a user's foot. The foot base 1800 can include certain elements that apply the aforementioned principles and techniques to generate electrical parameters, which can be correlated to physical parameters associated with a user's physical movements, when using the foot base 1800. Of course, according to other non-limiting aspects, the article can take the form of any other article of a sock, a midsole, an insole, or an outsole of a shoe, a mat, or a sheet, amongst others. According to the non-limiting aspect of FIG. 18, the article 1800 can be part of or included in any conventional or modern shoe design/structure.

In further reference to FIG. 18, the foot base 1800 can include a sole portion 1802. The sole portion 1802 can include a substrate 1812 and one or more flexible circuits comprising an electrical feature 1804_{a-d} electrically coupled via a network of traces 1806 that are specifically configured to traverse various geometrical portions of the foot base 1800. The foot base 1800 may further include an optional inertial measurement unit (IMU) 1808 and a coupling circuit 1810. According to some non-limiting aspects, any one of the foot base 1800, the traces 1806, the electrical features 1804_{a-d}, the IMU 1808, and/or the coupling circuit 1810 can be formed from a flexible and/or stretchable material. Accordingly, the foot base 1800, the traces 1806, the electrical features 1804_{a-d}, the IMU 1808, and/or the coupling circuit 1810 can enable the minimally inhibited motion of the user's foot while wearing or using the foot base 1800, and can be used to generate electrical parameters that can be correlated to physical parameters associated with physical movements or motions of the user (e.g., a position, an orientation, a speed, an acceleration, a weight transfer, a pose, and/or a prescribed sequence), as will be described further herein. According to some non-limiting aspects, the traces 1806 can be deposited onto the substrate 1812, or layups, of the foot base 1800 via the devices, systems, and methods disclosed in U.S. Provisional Patent Application No. 63/154,665, titled HIGHLY SUSTAINABLE CIRCUITS AND METHODS FOR MAKING THEM, filed Feb. 26, 2021, and/or International Patent Application No. PCT/US2019/047731 titled STRUCTURES WITH DEFORMABLE CONDUCTORS, filed Aug. 22, 2019, the disclosures of which are hereby incorporated by reference in its entirety.

For example, the traces 1806 can utilize flexible or highly elastic, deformable conductors or conductive systems, such as those disclosed in International Patent Application No. PCT/US2017/019762 titled LIQUID WIRE, which was filed on Feb. 27, 2017, and published on Sep. 8, 2017, as International Patent Publication No. WO2017/151523A1, the disclosure of which is hereby incorporated by reference in its entirety. For example, each trace 1806 can include a variety of forms, such as a liquid, a paste, a gel, and/or a powder, amongst others, that would enable the traces 1806 to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as a deformable conductor produced from a conductive gel (e.g., a gallium indium alloy based gel). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. The gallium oxide may be present within the bulk material as a crosslinked structure. Of course, the present disclosure contemplates other non-limiting aspects, featuring traces 1806 of varying forms and/or compositions to achieve the benefits disclosed herein.

According to some non-limiting aspects, the substrate 1812 may be composed of flexible and stretchable materials, such as those disclosed by U.S. patent application Ser. No. 16/548,379 titled STRUCTURES WITH DEFORMABLE CONDUCTORS, which was filed on Aug. 22, 2019, and granted as U.S. Pat. No. 11,088,063 on Aug. 10, 2021, the disclosure of which is hereby incorporated by reference in its entirety. Specifically, the substrate 1812 can be fabricated from a flexible or stretchable material such as a natural rubber, a synthetic rubber, a flexible plastic, a silicone based material (e.g., polydimethylsiloxane (“PDMS”), thermoplastic polyurethane (“TPU”), ethylene propylene dieneterpolymer (“EPDM”), neoprene, polyethylene terephthalate (“PET”), etc.), a flexible composite material, and/or a naturally flexible materials, such as a leather, for example. For example, the substrate 1812 can be fabricated from a resilient, albeit stretchable TPU, such as Lubrizol© Estane® 58000 series (e.g., 58238), amongst others. Alternatively, the substrate 1812 or at least a portion thereof can be formed from a flexible, though comparatively more rigid material, such as Lubrizol© Estane® S375D, flexible metal, fiberglass, or carbon fiber amongst others. For example, the substrate 1812 or at least a portion thereof may be composed of a carbon fiber material, such as those disclosed by International Patent Application No. PCT/US2021/072863 titled STRUCTURES WITH INTEGRATED CONDUCTORS, which was filed on Dec. 10, 2021, and published on Jun. 16, 2022, as International Patent Publication No. WO2022/126135A1, the disclosure of each of which is hereby incorporated by reference in its entirety. Although the substrate 1812 of FIG. 18 can include a multi-layer construction-including a substrate layer, a stencil-layer, and an encapsulation layer—in other non-limiting aspects, the substrate 1812 can include a two-layer construction (e.g., substrate layer, encapsulation layer, etc.) or even a single layer configured to accommodate the traces 1806. According to some non-limiting aspects, the substrate 1812 can be made with any suitable compressible material (e.g., flexible, rigid with some flexibility, or deformable). For example, the substrate 1812 may be a rigid plate that may be bent/deformed/compressed in response to a motion of a user (e.g., weight transfer) that stands on it.

Still referring to FIG. 18, the flexible and/or stretchable nature of the foot base 1800, the traces 1806, the electrical features 1804_{a-d}, the IMU 1808, and/or the coupling circuit 1810 can enable the generation of electrical parameters that can be correlated to physical parameters associated with physical movements of the user. For example, as the user wears or steps on the foot base 1800 and moves on or with the foot base, the resulting physical disturbance to the traces 1806, the electrical features 1804_{a-d}, the IMU 1808, and/or the coupling circuit 1810 mounted to or in the substrate 1812 can subsequently vary the electrical parameters (e.g., an inductance, a resistance, a voltage drop, a capacitance, and an electromagnetic field, etc.) generated by the traces 1806, the electrical features 1804_{a-d}, the IMU 1808, and/or the coupling circuit 1810. In other words, the user's motions while using the foot base 1800 can result in deformation of the traces 1806, the electrical features 1804_{a-d}, the IMU 1808, and/or the coupling circuit 1810 that will alter electrical parameters that can be correlated to baseline data—which can be gathered using methods that will be discussed in further detail herein—to monitor and/or characterize the user's motion (e.g., a position, an orientation, a speed, an acceleration, a weight transfer, a pose, and/or a prescribed sequence, such as walking, jumping, crouching, etc.) while using the foot base 1800. The electrical parameters (e.g., an inductance, a resistance, a voltage drop, a capacitance, and an electromagnetic field, etc.) generated by the traces 1806, the electrical features 1804_{a-d}, the IMU 1808, and/or the coupling circuit 1810 can be correlated to physical parameters (e.g., a strain, a stress, a pressure, a dimension, etc.) associated with the electrical features 1804_{a-d}, the IMU 1808, and/or the coupling circuit 1810, and thus, can characterize the user's motion while wearing or using the foot base 1800. The differences in correlated physical parameters of each circuit 1804_{a-d} can be used to model the user's motion in a virtual environment.

The electrical features 1804_{a-d} and/or the IMU 1808 may generate signals that can be correlated to physical parameters of the foot base 1800 (e.g., gyroscopes, accelerometers, magnetometers, pressure sensors, etc.). According to other non-limiting aspects, a micro-electrical mechanical system (“MEMS”) gyroscope could also be employed.

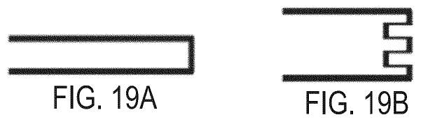

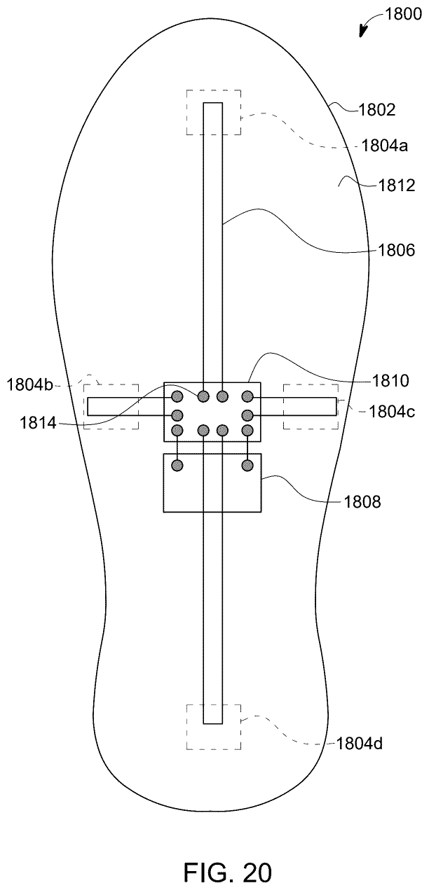

According to some non-limiting aspects of FIG. 18, the electrical features 1804_{a-d} comprise a deformable conductor that is arranged to form a pattern of traces. The pattern of traces may be at least one trace having some particular configurations or shapes. Some examples of the particular trace configurations or shapes are illustrated in FIGS. 19A and 19B. Additional descriptions of the trace patterns are disclosed in International Patent Application No. PCT/US2017/019762 titled LIQUID WIRE, which was filed on Feb. 27, 2017, and published on Sep. 8, 2017, as International Patent Publication No. WO2017/151523A1, the disclosure of which is hereby incorporated by reference in its entirety. FIG. 20 illustrates a foot base 1800 when the electrical features 1804_{a-d} of the foot base 1800 have a trace pattern shown in FIG. 19A. Of course, according to other non-limiting aspects, other geometric arrangements for the electrical features 1804_{a-d}/particular trace patterns can be implemented. In some non-limiting aspects, each electrical feature 1804_{a-d} may have a different geometric arrangement than the rest of the traces 1806.

In some non-limiting aspects, the electrical features 1804_{a-d}/trace patterns may include or serve as a strain sensor. More descriptions about a strain sensor are disclosed in International Patent Application No. PCT/US2017/019762 titled LIQUID WIRE, which was filed on Feb. 27, 2017, and published on Sep. 8, 2017, as International Patent Publication No. WO2017/151523A1, U.S. patent application Ser. No. 16/548,379 titled STRUCTURES WITH DEFORMABLE CONDUCTORS, which was filed on Aug. 22, 2019, and granted as U.S. Pat. No. 11,088,063 on Aug. 10, 2021, and U.S. Provisional Patent Application No. 63/261,266, titled STRETCHABLE AND FLEXIBLE METAL FILM STRUCTURES, filed Sep. 21, 2021, the disclosures of which are hereby incorporated by reference in their entirety. The deformable conductor and/or the pattern of traces may be configured to change its viscosity in response to a strain applied, by the user input, to the flexible circuits/electrical features 1804_{a-d}.

In some non-limiting aspects, the trace 1806 employed by the foot base 1800 can include a series of “switch backs,” wherein the trace 1806 loops back on itself, thereby extending the length of the trace 1806 in that particular portion of the foot base 1800 (e.g., where the electrical features 1804_{a-d} are located). The portions of the foot base 1800 where the electrical features 1804_{a-d}/particular trace patterns are positioned may be of specific interest to the user. For example, according to the non-limiting aspect of FIG. 18, the electrical features 1804_{a-d}/particular trace patterns can be positioned at approximately an estimated position of a user's foot when wearing or using the foot base 1800. For example, the electrical features 1804_{a-d} can be positioned in front, back, left, and/or right portions of the sole portion 1802 of the foot base 1800. The front, back, left, and right portions of the sole portion 1802 may be configured to be associated with the corresponding portions (e.g., front, back, left, and right portions) of a sole of the user, respectively. In other non-limiting aspects, the electrical features 1804_{a-d}/particular trace patterns can be positioned in any other suitable portions of the foot base 1800 (e.g., front-left, front-right, back-left, back-right, center, etc.). Although four electrical features are illustrated in FIG. 18, there could be more than or less than four electrical features (e.g., 1, 2, 3, 5, 6, 7, . . . ) in the foot base 1800.

In some non-limiting aspects, each electrical feature 1804_{a-d} and the trace 1806 (e.g., deformable conductor) connected to the respective electrical feature 1804_{a-d} may form a flexible circuit. The flexible circuit may further include a substrate layer and an encapsulation layer covering the trace 1806/electrical feature 1804_{a-d}. In some non-limiting aspects, the substrate layer of the flexible circuit may be the substrate 1812 of the foot base 1800. In other non-limiting aspects, the substrate layer of the flexible circuit may be independent of and separate from the substrate 1812 of the foot base 1800 and placed on or in the substrate 1812 of the foot base 1800. In some non-limiting aspects, a portion of the trace 1806 may form the electrical feature 1804_{a-d}.

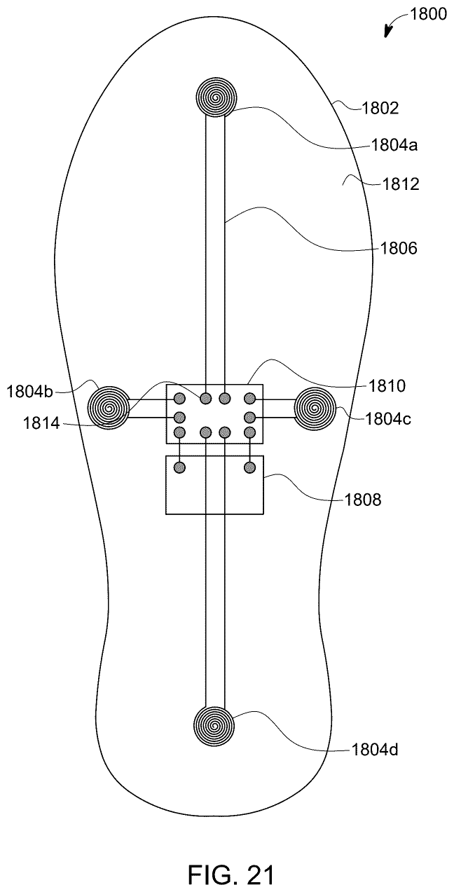

In some non-limiting aspects, the flexible circuits/electrical features 1804_{a-d} may include or serve as a pressure sensor (e.g., inductive coil sensor). FIG. 21 shows an example foot base with the pressure sensors 1804_{a-d}. More descriptions of a pressure sensor are disclosed in U.S. patent application Ser. No. 17/192,725, titled DEFORMABLE INDUCTORS, filed Mar. 4, 2021, and/or PCT Patent Application No. PCT/US2021/071374, titled WEARABLE ARTICLE WITH FLEXIBLE INDUCTIVE PRESSURE SENSOR, filed Sep. 3, 2021, the disclosures of both which are hereby incorporated by reference in their entirety. For example, some portions of the trace patterns of the electrical features 1804_{a-d} may have an inductive coil form. The flexible circuits/electrical features 1804_{a-d} may further include a conductor and a spacer between the inductive coil trace patterns and the conductor. The spacer may be resiliently deformable or have the characteristics of a spring to allow the spacer to compress when force is applied to one or both of the inductive coil and the conductor. In some non-limiting aspects, the conductor may be part of the trace patterns. In other non-limiting aspects, the conductor may be separate from the trace patterns.

In some non-limiting aspects, the flexible circuits/electrical features 1804_{a-d} may include or be part of a fluid-fillable circuit (or a partially fluid-fillable circuit). For example, the flexible circuits/electrical features 1804_{a-d} may include or be part of the fluid-fillable circuits (or partially fluid-fillable circuits) discussed in reference to FIGS. 1, 4, 6, 8, 10, 12, 14, and 15. Additional examples of the fluid-fillable circuits/partially fluid-fillable circuits are disclosed in U.S. Provisional Patent Application No. 63/272,487, titled DEVICES, SYSTEMS, AND METHODS FOR MAKING AND USING A FLUID-FILLABLE CIRCUIT, filed Oct. 27, 2021, and U.S. Provisional Application No. 63/366,778, titled DEVICES, SYSTEMS, AND METHODS FOR MAKING AND USING A PARTIALLY FLUID-FILLABLE CIRCUIT, filed Jun. 22, 2022, the disclosures of both of which are hereby incorporated by reference in its entirety. For example, in some non-limiting aspects, the entire portion or part of the substrate 1812 may be configured as a fluid fillable circuit (e.g., a bladder or air bag) with layups (e.g., outer body frame of the substrate) and an internal cavity (e.g., formed inside the substrate 1812) that is fillable with a fluid. In other non-limiting aspects, each flexible circuit/electrical features 1804_{a-d} may include a fluid-fillable circuit (or a partially fluid-fillable circuit) placed in or on the substrate 1812. The traces/trace patterns in the electrical features 1804_{a-d} (e.g., traces 104, 204, 304, 404, 1004, 1204, 1404, 1504) may be part of the fluid fillable circuits. In this way, the flexible circuit/trace patterns may be operatively coupled to the cavity that is fillable with a fluid.

In some non-limiting aspects, the flexible circuits/electrical features 1804_{a-d} may include or be part of a fluid-fillable circuit that may have an inductive coil configuration. For example, the flexible circuits/electrical features 1804_{a-d} may include or be part of the fluid-fillable circuit 400 illustrated in FIG. 8. As shown in FIG. 8, the traces 404ₐ can form a multi-layer or multi-level coil in a layup, e.g., the layup 402ₐ. The coiled trace 404ₐ can have a depth, meaning the coils descend towards the substrate layer of the layup 402ₐ, in some embodiments forming an Escher-like staircase configuration. The coil may be constructed using the principles disclosed in U.S. Provisional Application No. 63/270,589, titled FLEXIBLE THREE-DIMENSIONAL ELECTRONIC COMPONENT, filed Oct. 22, 2021, the disclosure of which is hereby incorporated by reference in its entirety. Since the trace 404ₐ of FIG. 8 is arranged in a coiled configuration, a current may generate a particular inductive and/or electromagnetic response as it is applied to the circuit 400 and traverses the trace 404ₐ. Still referring to FIG. 8, a conductive layer 418 may be provided and can be mechanically coupled to an outer surface 415, an inner surface, or laid up in between any of the layers of the second layup 402_{b}. Collectively, the coiled trace 404ₐ and conductive layer 418 can function as an inductive sensor configured to utilize inductive responses generated by the coiled trace 404ₐ to determine a relative distance between the coiled trace 404ₐ on the first layup 402ₐ and the conductive layer 418 on the second layup 402_{b}. The trace 404ₐ and/or the conductive layer in FIG. 8 can be part of the flexible circuit/electrical feature 1804ₐ-d/trace pattern.

In some non-limiting aspects, the electrical features 1804_{a-d}/trace patterns may be and/or include any suitable electrical component/sensor, such as a strain sensor, a pressure sensor, and a sensitive resistor. In some non-limiting aspects, the electrical features 1804_{a-d}/trace patterns/traces 1806 may be attached/connected to any suitable electrical component/sensor, such as a strain sensor, a pressure sensor, and a sensitive resistor.

In some non-limiting aspects, the IMU 1808 may be positioned in approximately the center portion of the insole portion 1802 of the foot base 1800. In other non-limiting aspects, the IMU 1808 can be positioned in any other suitable portions of the food base 1800 (e.g., front, back, left, right portion, etc.). The IMU 1808 can be configured to generate signals, which—according to some non-limiting aspects, in conjunction with signals generated by one or more other circuits 1804_{a-d}—can be correlated to physical parameters of the foot base 1800 and used to characterize a user's motions when wearing or using the foot base 1800. In some non-limiting aspects, the IMU 1808 of FIG. 18 can include a number of accelerometers, which can output linear acceleration signals on three axes in space, and/or gyroscopes, which can output angular velocity signals on three axes in space, to measure triaxial acceleration and/or angular velocity of the user's hand while wearing the foot base 1800. It shall be further appreciated how, in conjunction with the other circuits 1804_{a-d}, the IMU 1808 can be used to determine other aspects of a position and orientation of the foot base 1800 in three-dimensional space. For example, the various traces 1806 and electrical features 1804_{a-d} of the flexible circuits can generate electrical parameters (e.g., an inductance, a resistance, a voltage drop, a capacitance, and an electromagnetic field, etc.), which can be used to contextualize and/or calibrate signals generated by the IMU 1808. If the IMU 1808 begins to drift due to extended use, a processor communicably coupled to the circuits 1804_{a-d} can utilize signals associated with electrical parameters from the other circuits 1804_{a-d} to correct signals received from the IMU 1808.

According to some non-limiting aspects, the IMU 1808 can include an onboard construction, including traces that are constructed of a deformable conductor, similar to the traces of the individual circuits 1804_{a-d}. As such, deformations within the IMU 1808 itself can be utilized to contextualize and/or calibrate signals generated by other components IMU 1808 (e.g., gyroscopes, accelerometers, magnetometers, pressure sensors, etc.). In other words, according to some non-limiting aspects, the IMU 1808 can be constructed according to U.S. Provisional Patent Application No. 63/261,266, titled STRETCHABLE AND FLEXIBLE METAL FILM STRUCTURES, filed Sep. 21, 2021, to reduce the need for additional circuits 1804_{a-d}.

In further reference to FIG. 18, the coupling circuit 1810 can be provided to couple the flexible circuits/electrical features 1804_{a-d} and the traces 1806 of the foot base 1800 to a processing circuit via a plurality of vias 1814, such as those disclosed in U.S. Provisional Patent Application No. 63/261,266, titled STRETCHABLE AND FLEXIBLE METAL FILM STRUCTURES, filed Sep. 21, 2021, the disclosure of which is hereby incorporated by reference in its entirety. For example, the traces 1806, vias 1814, and contacts (not shown) may be particularly sized and spaced to establish the desired electrical connections such that signals generated by the circuits 1804_{a-d} of the foot base 1800 can be transmitted to a processor, for example, via an electrical connector. Of course, according to other non-limiting aspects, the coupling circuit 1810 can be hardwired to the processor. The processor can be communicably coupled to a memory configured to store instructions that, when executed by the processor, cause the processor to characterize the user's motion while wearing or using the foot base 1800. The processor can be coupled to a display that can be configured to present a virtual representation of the user's movements in a virtual environment. Alternately, the coupling circuit 1810 can be configured for conventional wireless (e.g., infrastructure networks, such as WiFi®, cellular, etc., and/or ad hoc networks, such as Bluetooth®, near-field communication (“NFC”), RFID) transmissions. According to some non-limiting aspects, the processor can be remotely located relative to the foot base 1800.

According to some non-limiting aspects, a display may be provided on/in the foot base 1800 and the processor may not be coupled to the foot base 1800. For example, electrical signals sensed/generated by the foot base 1800 may be transmitted wirelessly to a cloud for processing and data/instructions may be transmitted wirelessly from the cloud to the foot base 1800, for example, to the display on the foot base 1800, thereby displaying the data/instructions on the display coupled to the foot base 1800. In other non-limiting aspects, the display may be placed outside of the foot base 1800.

According to other non-limiting aspects, the coupling circuit 1810 of the foot base 1800 can further include an on-board processor such that signals generated by the circuits 1804_{a-d} can be locally processed and the coupling circuit 1810 can couple the foot base 1800 to the display. In some non-limiting aspects, the coupling circuit 1810 can include a rechargeable power source (e.g., a lithium-ion battery, a capacitor, etc.) configured to deliver an electrical current to the circuits 1804_{a-d} and/or a port (e.g., a universal serial bus (“USB”) port) configured to directly deliver an electrical current to the circuits 1804_{a-d} and/or charge the power source, itself.

Still referring to FIG. 18, an additional substrate (other than the substrate 1812) can be provided under the electrical features 1804_{a-d}, the IMU 1808, and/or the coupling circuit 1810 (or part of these circuits). This additional substrate can be fabricated from a more resilient, albeit stretchable, TPU, such as Lubrizol© Estane® 58000 series (e.g., 58238), amongst others. Alternatively, the additional substrate or at least a portion thereof can be formed from a flexible, though comparatively more rigid, material, such as Lubrizol© Estane® S375D, flexible metal, fiberglass, or carbon fiber, amongst others. For example, the additional substrate or at least a portion thereof may be composed of a carbon fiber material, such as those disclosed by International Patent Application No. PCT/US2021/072863 titled STRUCTURES WITH INTEGRATED CONDUCTORS, which was filed on Dec. 10, 2021, and published on Jun. 16, 2022, as International Patent Publication No. WO2022/126135A1, the disclosure of each of which is hereby incorporated by reference in its entirety. Accordingly, the additional substrate can be reinforced to limit and/or restrict deformations of the electrical features 1804_{a-d}, the IMU 1808, and/or the coupling circuit 1810 altogether, or in a particular axis, such that electrical parameters do not vary as much relative to other traces 1806 and/or the electrical features 1804_{a-d}, the IMU 1808, and/or the coupling circuit 1810 of interest. In other words, the relative flexibility and rigidity of various portions and/or components of the foot base 1800 can be used to ensure signals generated by the circuits 1804_{a-d} carry information relevant to areas of interest. This can lead to more efficient processing and, thus, enhance the accuracy and economic value of characterizations generated by the foot base 1800.

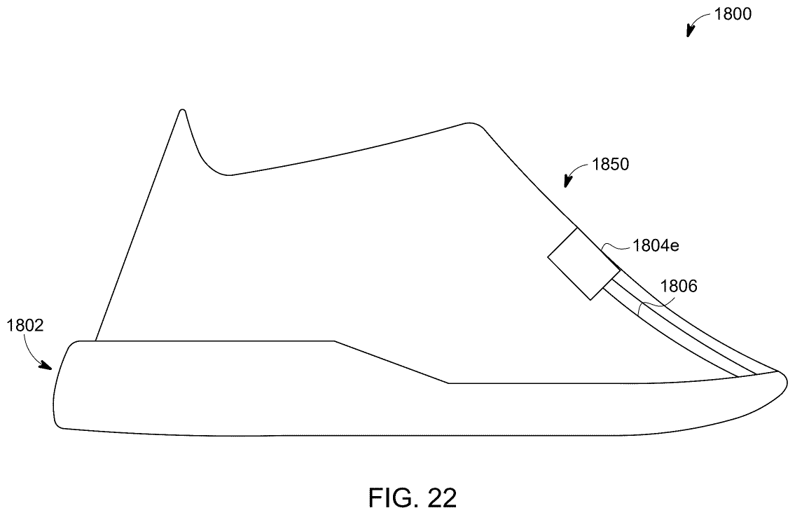

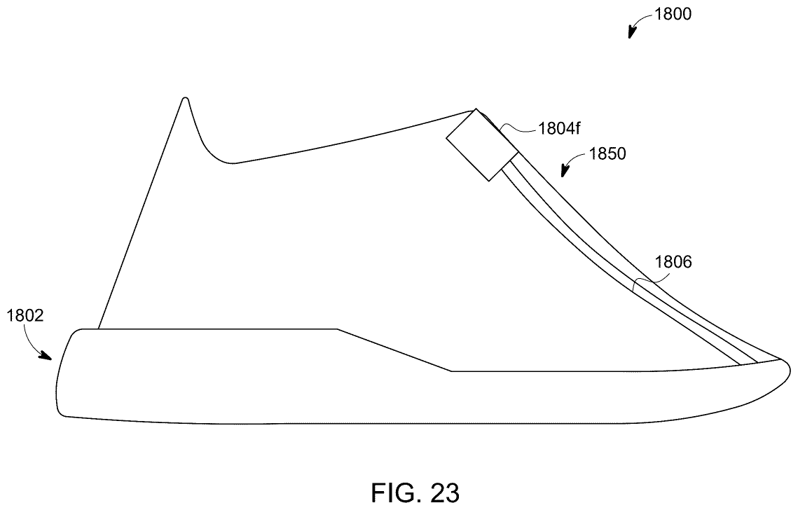

Referring now to FIGS. 22 and 23, in some non-limiting aspects, the foot base 1800 may further include an upper portion 1850 in addition to the sole portion 1802. The upper portion 1850 may be configured to surround an instep and/or an ankle of the user. In some non-limiting aspects, the foot base 1800 may further include a flexible circuit (e.g., an electrical feature and a trace 1806) placed on or in the upper portion 1850 of the foot base 1800. For example, an electrical feature 1804ₑ can be placed in or on an instep portion of the upper portion 1850 as shown in FIG. 22. The instep portion may be configured to be associated with the instep of the user. The electrical feature 1804ₑ in/on the instep portion may detect the force/pressure/strain applied to the instep portion (and ultimately the instep of the user), which can be used to monitor the stress applied to the instep portion. For example, when a user is kicking a punching sandbag (for example, while playing a virtual fighting game), the amount of force/pressure applied to the instep portion and/or the punching sandbag can be detected through the electrical feature 1804ₑ.

In some other examples, an electrical feature 1804f can be placed in or on an ankle portion of the upper portion 1850 as shown in FIG. 23. The ankle portion may be configured to be associated with the ankle of the user. The electrical feature 1804f in the ankle portion may be able to detect dorsiflexion and/or plantar flexion of a foot of the user, thereby improving the accuracy of the detection of the user's motion (e.g., weight transfer, poses, etc.).

The flexible circuits (e.g., electrical features 1804_{e-f} and the associated traces 1806) may be coupled to the coupling circuit 1810. In some examples, an IMU (e.g., the IMU 1808 or a separate IMU) and/or a coupling circuit (e.g., the coupling circuit 1810 or a separate coupling circuit) can be placed in the upper portion 1850 of the foot base 1800. In some examples, the upper portion 1850 may be configured similar to the substrate 1812 of the sole portion 1802 (e.g., materials, configurations, features, etc.). In other examples, the upper portion 1850 may be configured with any other suitable way (e.g., having a substrate made with a material that is normally used for making an upper portion of a shoe). Other configurations/features/characteristics of the flexible circuits/electrical features 1804_{e-f}, IMU, coupling circuit, and/or upper portion (e.g., materials, functions, structures, components, such as strain sensor, pressure sensor, fluid fillable circuit, vias, and substrate, etc.) may be similar to and/or same as the ones described above with respect to the foot base 1800 of FIG. 18, and, thus, duplicate description may be omitted.

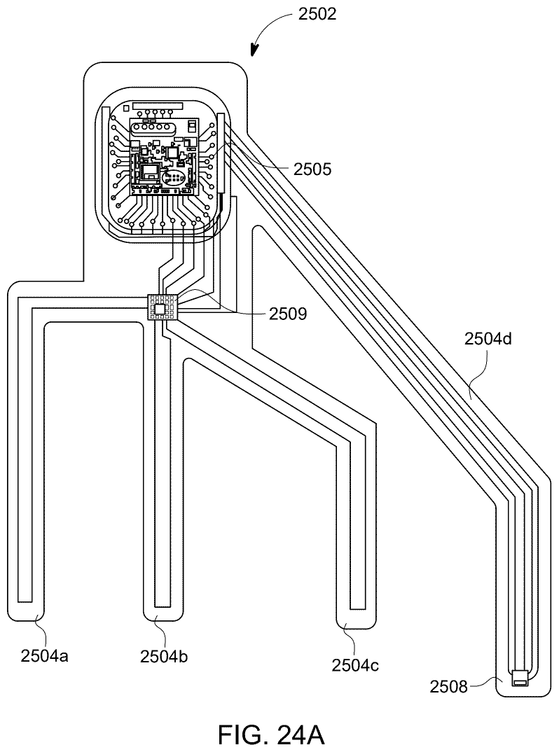

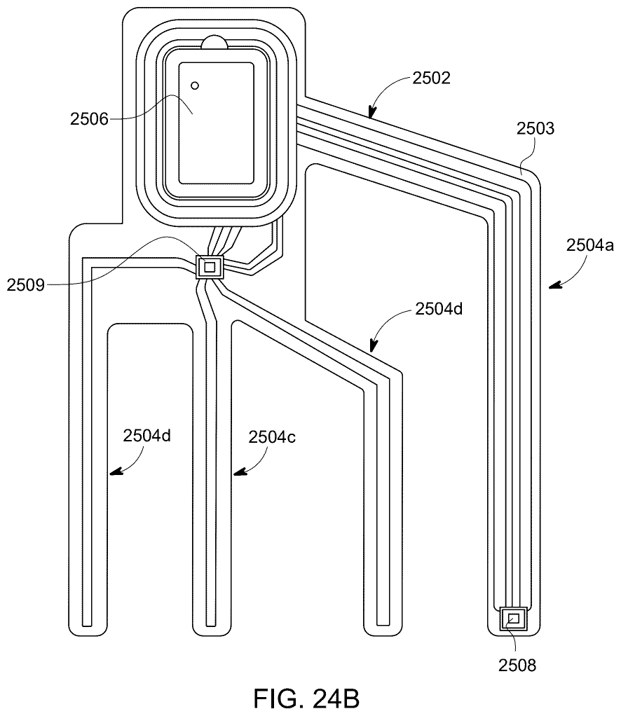

In some non-limiting aspects, any suitable electrical feature/component can be disposed/located on and/or in the upper portion 1850. Examples of such electrical feature/component may include a power source, an output device, any of the sensors discussed in the present application, and/or a combination thereof, for example, as described in U.S. Provisional Patent Application No. 63/412,867 titled DEVICES, SYSTEMS, AND METHODS FOR INTERACTING WITH & CALIBRATING A WEARABLE ARTICLE FEATURING FLEXIBLE CIRCUITS, which was filed on Oct. 3, 2022, the disclosure of which is hereby incorporated by reference in its entirety. For example, referring now to FIG. 24A, a flexible circuit 2502 configured for use with a wearable article 2500 (e.g., upper portion 1850) is depicted in accordance with at least one non-limiting aspect of the present disclosure. According to the non-limiting aspect of FIG. 24, the flexible circuit 2502 can include one or more traces 2504a-d defined by a deformable conductor. For example, according to some non-limiting aspects, the flexible circuit 2502 can be constructed as disclosed in U.S. Provisional Patent Application No. 63/154,665, titled HIGHLY SUSTAINABLE CIRCUITS AND METHODS FOR MAKING THEM, filed Feb. 26, 2021, and/or International Patent Application No. PCT/US2019/047731 titled STRUCTURES WITH DEFORMABLE CONDUCTORS, filed Aug. 22, 2019, the disclosures of which are hereby incorporated by reference in their entirety.

Additionally, the traces of a flexible circuit can be constructed from a fluid-phase conductor. As used herein, the term “fluid-phase conductor” shall include any of the flexible, deformable conductors described herein and/or any of the flexible, deformable conductors described in any document incorporated by reference. Specifically, “fluid-phase conductors” are described in International Patent Application No. PCT/US2017/019762 titled LIQUID WIRE, which was filed on Feb. 27, 2017 and published on Sep. 8, 2017 as International Patent Publication No. WO2017/151523A1, and/or International Patent Application No. PCT/US2019/047731 titled STRUCTURES WITH DEFORMABLE CONDUCTORS, filed Aug. 22, 2019, the disclosures of which are hereby incorporated by reference in their entirety.

For example, according to some non-limiting aspects, each trace can include a variety of forms, such as a liquid, a paste, a gel, and/or a powder, amongst others that would enable the traces to have a deformable (e.g., soft, flexible, stretchable, bendable, elastic, flowable viscoelastic, Newtonian, non-Newtonian, etc.) quality. According to some non-limiting aspects, the deformable, conductive materials can include an electroactive material, such as deformable conductors produced from a conductive gel (e.g., a gallium indium alloy). The conductive gel can have a shear thinning composition and, according to some non-limiting aspects, can include a mixture of materials in a desired ratio. For example, according to one preferable non-limiting aspect, the conductive gel can include a weight percentage of a eutectic gallium alloy between 59.9% and 99.9% and a weight percentage of a gallium oxide between 0.1% and about 2.0%. Of course, the present disclosure contemplates other non-limiting aspects, featuring traces of varying forms and/or compositions to achieve the benefits disclosed herein.

The electrically conductive compositions can be characterized as conducting shear thinning gel compositions. The electrically conductive compositions described herein can also be characterized as compositions having the properties of a Bingham plastic. For example, the electrically conductive compositions can be viscoplastics, such that they are rigid and capable of forming and maintaining three-dimensional features characterized by height and width at low stresses but flow as viscous fluids at high stress. According to other non-limiting aspects, the low-shear viscosity of useful metal gel can be 106 to 4×107 Pa*s (1,000,000-40,000,000 Pascal seconds), wherein “low-shear” viscosity refers to a viscosity at rest (or sedimentation) conditions. The micro/nanostructure comprises oxide sheets that form a cross-linked structure, which may be achieved e.g. by mixing in a way that entrains air into the mixture, or by sonication that induces cavitation at the surface drawing in air to the mixture such that oxide formation in the cross-linked structures can be achieved.

It shall be appreciated that, by using flexible circuits and deformable conductors, various sensors can be constructed that, when integrated into a wearable article (e.g., upper portion 1850) worn by a user, can generate varying electrical parameters (e.g., an inductance, a resistance, a voltage drop, a capacitance, and an electromagnetic field, etc.) that can be correlated to physical parameters (e.g., a strain, a stress, a pressure, a dimension, etc.) and thus, used to generate highly accurate simulations of the user's motions while wearing the article. For example, a wearable article can utilize flexible circuits and deformable conductors configured to function as sensors (e.g., a strain sensor, etc.). Enabled by the deformable conductor, which is configured to move with the joint, a wearable article can actively and accurately monitor joint flexibility without substantial electrical or physical degradation over thousands of strain cycles. Accordingly, continuous calibration is unnecessary and conversely, the flexible circuits can be used to calibrate conventional sensors (e.g., IMUs, etc.).

Additionally, it shall be appreciated that the flexible circuit 2502 of FIG. 24 can include a flexible substrate 2503, which can be constructed via one or more flexible layers. For example, the substrate 2503 can be constructed as a laminate structure that incorporates at least one layer onto which conductive gel is positioned to form the traces 2504a-d. The layers can include at least one substrate layer that forms a foundation for at least one trace 2504a-d, and at least one encapsulation layer that seals the trace or other component of the laminate structure. According to other non-limiting aspects, the laminate structure may further include: a stencil layer, e.g., for when a stencil-in-place manufacturing process is utilized; a conductive layer for, e.g., a relatively high-powered bus, sensor, ground plane, shielding, etc.; an insulation layer, e.g., between a substrate layer, a conductive layer, a stencil layer, and/or an encapsulation layer, that primarily insulates traces or conductive layers from one another; an electronic component not necessarily formed according to the processes disclosed herein, e.g., a surface mount capacitor, resistor, processor, etc.; vias for connectivity between layers; and contact pads.

The collection of layers of the laminate structure may be referred to as a “stack”. A final or intermediate structure may include at least one stack (or multiple stacks, e.g., using modular construction techniques) that has been unitized. Additionally or alternatively, the structure could comprise one or more unitized stacks with at least one electronic component. A laminate assembly may comprise multiple laminate structures, e.g., in a modular construction. The assembly may utilize island architecture including a first laminate structure (the “island”), which may typically but not exclusively be itself a laminate structure populated with electric components, or a laminate structure that is, e.g., a discrete sensor, with the first laminate structure adhered to a second laminate structure including, e.g., traces and vias configured like a traditional printed circuit board (“PCB”), e.g., acting as the pathways for signals, currents or potentials to travel between the island(s) and other auxiliary structures, e.g., sensors.

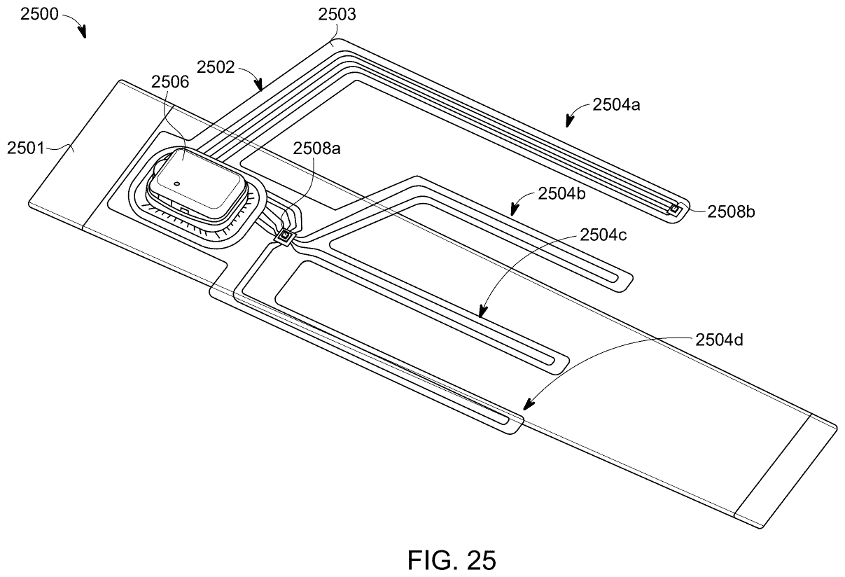

In further reference to FIG. 24A, the flexible circuit 2502 can further include an analog-to-digital converter (“ADC”) 2509 electrically coupled to one or more of the traces 2504a-d and an electronic component 2505, such as an integrated circuit that includes surface-mounted processor or microprocessor. It shall be appreciated that the ADC 2509 can be configured to convert analog signals generated across one or more of the traces 2504a-d and provide them to the electronic component 2505 for onboard processing and/or transmission. For example, according to some non-limiting aspects, the electronic component 2505 can include a microprocessor (e.g., a Nordic-brand nRF MDK-based processor or equivalent, etc.), a memory, a wireless communication circuit, and/or a bus port (configured to receive power and/or data from the power component 2506 of FIG. 24B), an additional IMU, additional sensors, etc. According to some non-limiting aspects, the ADC 2509 can be positioned on the electronic component 2505 of FIG. 25. The electronic component 2505 of FIG. 24A can include (or be surrounded by) a mechanical component, such as a cradle, configured to removably secure the power component 2506 of FIG. 24B to the wearable article 2500 of FIG. 25, and establish electrical communication between the bus component 2506 and the bus port of the electronic component 2505. Accordingly, when the power component 2506 of FIG. 24B is mechanically secured to the wearable article 2500 (FIG. 25) via the cradle, it can provide power and/or data to the electronic component 2505 of FIG. 24A.

With specific reference to FIG. 24B, the power component 2506 is depicted as mechanically and electrically coupled to the electronic component 2505 of FIG. 24A. As previously described, the power component 2506 can include a battery and/or charger. According to some non-limiting aspects, the charger can include a universal serial bus (“USB”) port configured to convey electrical power and/or data to the power component 2506 from an external source. For example, the power component can be configured for such conveyance via a USB-A, USB-B, or USB-C protocol, although other means for power and/or data conveyance are contemplated by the present disclosure. According to other non-limiting aspects, the power component 2506 can include a wireless charging circuit and/or a wireless transmitter and/or receiver configured to wireless obtain power and data from external sources. Regardless, it shall be appreciated that the power component 2506, when mechanically and electrically coupled to the electronic component 2505 of FIG. 24A, can provide electrical power to the flexible circuit 2502. Additionally, via the power component 2506 of FIG. 24B, it shall be appreciated that data can be transmitted to and from the flexible circuit 2502. For example, according to some non-limiting aspects, the power component 2506 can be used to transmit a firmware update to a memory of the electronic component 2505 of the flexible circuit 2502 for execution by its microprocessor. Alternately, the power component 2506 can include a memory configured to store data generated by the wearable circuit 2502 for subsequent use and processing.

According to other non-limiting aspects, one or more of the components (e.g., microprocessor, memory, wireless circuit, ADC, IMU, other sensors, etc.) of the electronic component 2505 of FIG. 24A can be alternately positioned within the power component 2506 of FIG. 24B. Accordingly, some or all of the functionality provided by the electronic component 2405 of FIG. 24A can be modular and interchangeable amongst several flexible circuits and/or wearable articles. This can promote efficiency and reduce the expense associated with manufacturing the wearable article 2500 (FIG. 25), itself. According to some non-limiting aspects, the electronic components 2505 of FIG. 24A can include an RFID chip, or another means of identifying its identity to the power component 2506. Accordingly, if the power component 2506 includes one or more of the components and/or functions of the electronic component 2505 of FIG. 24A, the power component 2506 can identify which flexible circuit 2502 and thus, which wearable article 2500 (FIG. 25) it is coupled to. This can ensure accurate tagging of data, including the association of data with a specific user and/or patient.

According to the non-limiting aspect of FIGS. 24A and 24B, at least one trace 2504a can be configured to function as a data and/or power bus 2504a-d electrically coupling the electronic component 2505 to at least one of the IMUs 2508. One or more other traces 2504a-c can be configured as a strain sensor. Of course, any of the traces 2504a-c can be configured to monitor any of the aforementioned physical parameters by way of the varying electrical parameters they generate while the wearable article 2500 (FIG. 25) is in use. According to other non-limiting aspects, any of the traces 2504a-c can be multiplexed and therefore, configured to simultaneously function as a sensor and a data bus.

Still referring to FIGS. 24A and 24B, the flexible circuit 2502 is configured for implementation via the wearable article 2500 of FIG. 25. However, it shall be further appreciated that the particular trace 2504a-d and IMU 2508 configuration depicted in FIG. 24A is merely illustrative and can be specifically attenuated to monitor any particular body part and/or particular motions performed by a particular body part. Moreover, it shall be appreciated that other configurations of traces 2504a-d and one or more IMUs 2508 can be implemented to monitor alternate motions performed by a user.

Referring now to FIG. 25, a wearable article 2500 configured to use the flexible circuit 2502 of FIG. 24 is depicted in accordance with at least one non-limiting aspect of the present disclosure. According to the non-limiting aspect of FIG. 25, the wearable article 2500 can be configured to be disposed/located on and/or in the upper portion 1850. However, it shall be appreciated that, according to other non-limiting aspects, the wearable article 2500 can be alternately designed to be worn about any suitable part of the body of a user.

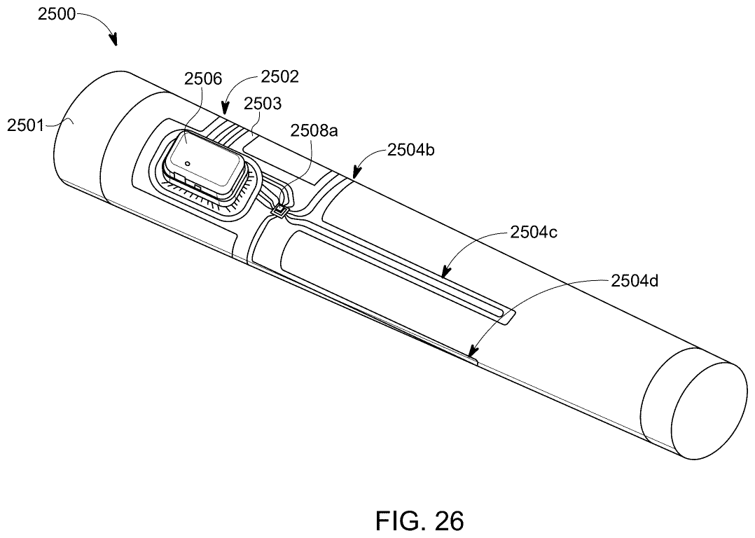

According to the non-limiting aspect of FIG. 25, the flexible circuit 2502 can be mounted, bonded, woven into, or otherwise secured to a flexible medium 2501 configured as a cylindrical tube. Of course, according to other non-limiting aspects, the flexible medium 2501 can be alternately configured to be worn in any particular fashion about any particular body part. As previously discussed, the flexible medium 2501 can be formed from elastic, spandex, cotton, and/or other natural and synthetic fabrics that provide the desired flexible and/or structural characteristics depending on a particular application and/or user preference. For example, according to the non-limiting aspect of FIG. 25, a portion of the flexible circuit 2502, including the electronic component 2505 and central sensing trace 2504c have been properly aligned on a particular portion of the flexible medium 2501 of the wearable article 2500 such that rest of the sensors 2504a, 2504b, 2504d and one or more IMUs 2508 the flexible circuit 2502 are positioned such that they monitor and measure the proper portion of the wearable article 2500 when worn. After the flexible circuit 2502 is properly aligned on the flexible medium 2501, the flexible circuit 2502 can be wrapped around an outer surface of the flexible medium 2501 and bonded such that the wearable article 2500—including the flexible circuit 2502—define a cylindrical structure as shown in FIG. 26.

Various numbers, configurations, and combinations of sensors made from flexible circuits and rotational sensors, such as IMUs can be implemented to capture any one of the motions described in the present application. Specifically, the traces 2504a-d (FIG. 24) of the flexible circuit 2500 (FIG. 24) can be implemented to monitor and characterize extensions and flexions while the one or more IMUs 2508 (FIG. 24) can be implemented to monitor and characterize rotational motions. As such, various combinations of traces 2504a-d (FIG. 24) and one or more IMUs 2508 (FIG. 24) can be implemented to monitor and characterize combined motions, including adduction and/or abduction of the user's foot.

In other words, according to the non-limiting aspect of FIG. 26, the wearable article 2500 of FIG. 25 can be configured to characterize motions related to the foot movements. However, according to some non-limiting aspects, the wearable article 2500 (FIG. 25) can be further configured to monitor and detect muscle activations related to foot, instep, and/or ankle movements, via the traces 2504a-d (FIG. 24).

In some non-limiting aspects, the above-discussed electrical feature/component can be disposed/located on and/or in any suitable portion of the device according to the present disclosure, including the upper portion 1850, the foot base 1800, or partially in/on the upper portion 1850 and partially in/on the foot base 1800.

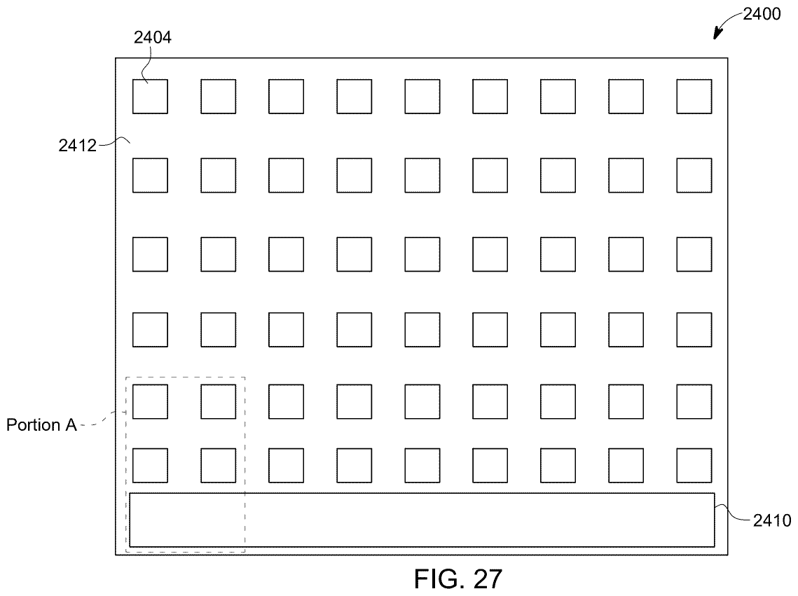

Referring now to FIG. 27, an article 2400 configured for the simulation of physical motions in a virtual environment is depicted in accordance with at least one non-limiting aspect of the present disclosure. For example, according to the non-limiting aspect of FIG. 24, the foot base 2400 can be configured as a mat or a sheet to be placed under a user's foot. The foot base 2400 can include certain elements that apply the aforementioned principles and techniques to generate electrical parameters, which can be correlated to physical parameters associated with a user's physical movements, when using the foot base 2400.

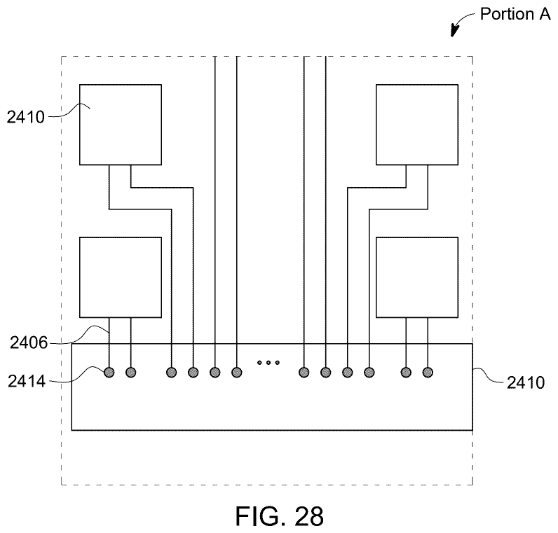

In further reference to FIG. 27, the foot base 2400 can include a substrate 2412 and one or more flexible circuits including an electrical feature 2404 electrically coupled via a network of traces 2406 that are specifically configured to traverse various geometrical portions of the foot base 2400. As shown in FIG. 27, in some non-limiting aspects, an array of electrical features may be placed throughout the mat/sheet 2400. FIG. 28 illustrates an enlarged view of the portion A of the foot base 2400 of FIG. 27. In some non-limiting aspects, the foot base 2400 may further include an IMU and a coupling circuit 2410. According to some non-limiting aspects, any one of the foot base 2400, the traces 2406, the electrical features 2404, and/or the coupling circuit 2410 can be formed from a flexible and/or stretchable material. According to some non-limiting aspects, the traces 2406 can be deposited onto the substrate 2412, or layups, of the foot base 2400 via the devices, systems, and methods disclosed in U.S. Provisional Patent Application No. 63/154,665, titled HIGHLY SUSTAINABLE CIRCUITS AND METHODS FOR MAKING THEM, filed Feb. 26, 2021, and/or International Patent Application No. PCT/US2019/047731 titled STRUCTURES WITH DEFORMABLE CONDUCTORS, filed Aug. 22, 2019, the disclosures of which are hereby incorporated by reference in its entirety.

According to some non-limiting aspects of FIG. 27, the electrical features 2404 include a deformable conductor that is arranged to form a pattern of traces. The pattern of traces may be at least one trace having some particular configurations or shapes. In some non-limiting aspects, each electrical feature 2404/trace pattern may serve as a strain sensor, pressure sensor (e.g., inductive coil sensor), and/or fluid-fillable circuit. In some non-limiting aspects, each electrical feature 2404 and the associated trace 2406 (e.g., deformable conductor) connected to the respective electrical feature 2404 may form a flexible circuit. The coupling circuit 2410 can be provided to couple the electrical feature 2404 and the associated trace 2406 of the mat/sheet 2400 to a processing circuit via a plurality of vias 2414. In some non-limiting aspects, the coupling circuit 2410 may be disposed in a bottom portion of the foot base 2400. In other non-limiting aspects, the coupling circuit 2410 can be placed in any other suitable portion of the foot base 2400 (e.g., top, left, right, or center portion of the foot base 2400). Although there are 54 electrical features 2404 illustrated in FIG. 27, there could be more or less electrical features 2404 placed on or in the substrate 2412 of the foot base 2400. Also, although an array of electrical features aligned vertically and horizontally is shown in FIG. 27, any other suitable arrangements of the electrical features 2404 are possible (e.g., zig-zag, circles, etc.).

As a user moves on the mat/sheet 2400, some of the flexible circuits/electrical features 2404 (e.g., the ones pressed by the user's foot or other portions of the body) may detect the pressure/strain/movements. The electrical signals (e.g., an inductance, a resistance, a voltage drop, a capacitance, and an electromagnetic field, etc.) generated from these flexible circuits/electrical features may be transmitted to a processor via the coupling circuit, and the processor may alter the virtual representation of the user (in a virtual environment) based on these signals. Based on the locations of the electrical features 2404 that are pressed by the user, the processor may be able to determine the location of the user's body on the mat/sheet 2400. Other configurations/features/characteristics of the foot base 2400 (e.g., materials, functions, structures, components of the flexible circuits/electrical features, IMU, coupling circuit, substrate, etc.) may be similar to and/or same as the ones described above with respect to the foot base 1800 of FIG. 18, and, thus, duplicate description may be omitted.

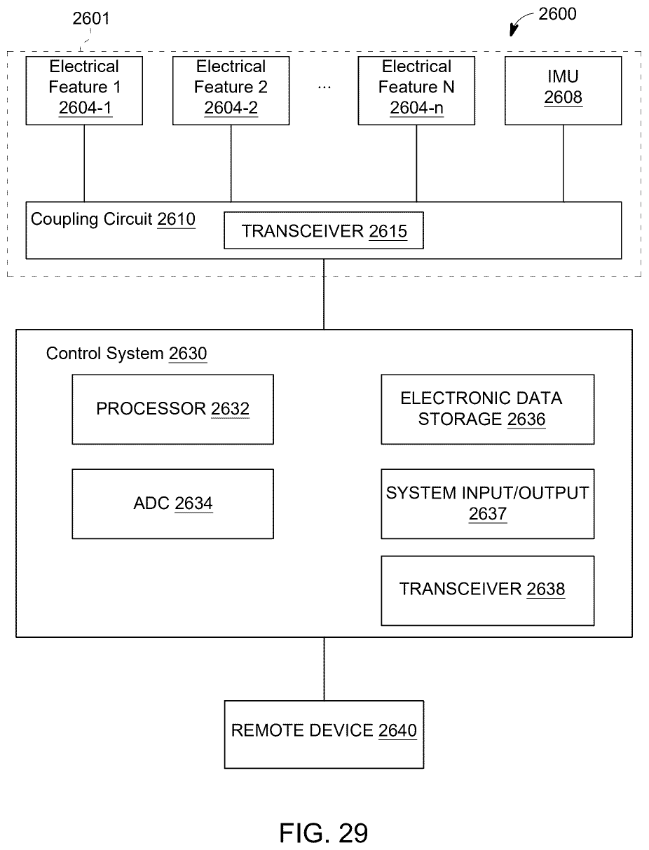

FIG. 29 is a block diagram of a foot base 2601 (e.g., the foot base 1800 or 2400) incorporated into a system 2600, in an example embodiment. It is to be recognized and understood that the system 2600 is provided for illustrative purposes and that no one component of the system is necessarily essential. Moreover, the incorporation of certain components in the system 2600 may be variable with respect to certain subsystems of the system 2600, and it is to be recognized and understood that certain components 2600 may be incorporated as part of any subsystem or omitted altogether.

The system 2600 may include a foot base 2601 (e.g., foot base 1800, 2400) having one or more flexible circuits/electrical features 2604-1-2604-n (e.g., electrical features 1804, 2404 and associated traces 1806, 2406) in a manner suitable for use in the system 2600 (e.g., physical dimensions, electrical characteristics). The foot base 2601 of the system 2600 may also include one or more IMUs 2608 (e.g., IMU 1808, 2408) and a coupling circuit 2610 (e.g., coupling circuit 1810, 2410). The coupling circuit 2610 can be provided to couple the flexible circuits/electrical features 2604-1-2604-n of the foot base 2601 to a control system 2630 of the system 2600. The coupling circuit 2610 may include a transceiver 2615 for transmitting/receiving signals to/from the control system 2630. The transceiver 2615 may be configured to be in communication with a transceiver 2638 of the control system 2630. The transceiver 2638 may be provided for receiving signals from the coupling circuit 2610.

The control system 2630 may further include a processor 2632, an analog to digital converter (ADC) 2634, an electronic data storage 2636, and a system input/output 2637. The processor 2632 may be a conventional processor, microprocessor, controller, microcontroller, or any suitable processing or controlling device. The processor 2632 may receive the output (e.g., an inductance, a resistance, a voltage drop, a capacitance, and an electromagnetic field, etc.) from the foot base 2601. The electronic data storage 2636 may be any one or more of a volatile or nonvolatile electronic data storage, such as memory, hard drive, cache, or the like. The processor 2632 can be communicably coupled to the electronic data storage 2636 configured to store instructions that, when executed by the processor 2632, cause the processor 2632 to characterize the user's motion while wearing or using the foot base 2601. The ADC 2634 may convert analog signals (e.g., signals from the foot base 2601) to digital signals for interpretation by the processor 2632. The system input/output 2637 may be provided to communicate outside of the system 2600. In some non-limiting aspects, some of the components of the control system 2630 may be included in the foot base 2601 (e.g., in the coupling circuit 2610).

The system 2600 may further include a remote device 2640 that may be controlled by the control system 2630 based at least in part on the output from the foot base 2601. The remote device 2640 may be any device or system that may utilize information obtained on the basis of output from the foot base 2601. In some non-limiting aspects, the remote device 2640 may be a data output device, such as a display screen that provides a virtual representation of the user's motion, for example, while the user is wearing/using the foot base 2601. The above examples of the remote device 2640 are provided by way of example, and it is to be recognized and understood that the remote device 2640 may be any device or system that may utilize the output of the foot base 2601. In some non-limiting aspects, the display may be placed on/in the foot base 2601 (e.g., on a top surface of a mat). The display may display any instructions or data from the processor/cloud.

The control system 2630 may include any additional components as desired to support the operation of the foot base 2601 and the system 2600 in general. The control system 2630 may include or be a part of a discrete computing device, such as a personal computer, smart phone, tablet computer, or the like and as such may include components of such a discrete computing device. In such an example, the foot base 2601 may include a wired or wireless connection to the discrete computing device. Additionally or alternatively, the control system 2630 may be, may include, or may access cloud computing resources or other remote computing resources. Moreover, the remote device 2640 may be or may include the discrete computing device or cloud computing resources. The various components of the system 2600 may be operatively coupled with respect to one another by wired or wireless technologies.

In some non-limiting aspects, the processor 2632 may receive a signal from a flexible circuit/electrical feature 2604, determine an electrical parameter based on the received signal, correlate the determined electrical parameter to a physical parameter of the flexible circuit/electrical feature 2604, and alter the virtual representation of the user (e.g., displays in the remote device 2640) based on the correlation.

In some non-limiting aspects, the system 2600 may include a plurality of flexible circuits, for example, a first flexible circuit and a second flexible circuit. The first flexible circuit may include a first electrical feature 2604-1 and a first deformable conductor (e.g., a first trace associated with the first electrical feature 2604-1) electrically coupled to the first electrical feature 2604-1. The first electrical feature 2604-1 may be positioned in a first location of interest on the foot base 2601. The second flexible circuit may include a second electrical feature 2604-2 and a second deformable conductor (e.g., a second trace associated with the second electrical feature 2604-2) electrically coupled to the second electrical feature 2604-2. The second electrical feature 2604-2 may be positioned in a second location of interest on the foot base 2601. In some non-limiting aspects, the processor 2632 may receive first and second signals from the first and second flexible circuits, respectively, determine first and second electrical parameters based on the first and second signals, respectively, and correlate the first and second parameters to first and second physical parameters, respectively. The first and second physical parameters may be associated with the first and second locations of interest, respectively. Then, the processor 2632 may compare the first and second physical parameters with each other and alter the virtual representation of the user based on the comparison of the first and second physical parameters associated with the first and second locations of interest. For example, when the foot base 2601 is the foot base 1800 of FIG. 18, the first and second locations of interest may be one of front, back, left, and right portions of the sole portion 1802 of the foot base 1800. The front, back, left, and right portions of the sole portion 1802 are configured to be associated with front, back, left, and right portions of a sole of the user, respectively.

In some non-limiting aspects, the system 2600 may use more than two flexible circuits to generate/alter the representation of the user in a virtual environment. For example, the foot base 2601 may further include a third flexible circuit and/or a fourth flexible circuit. The third flexible circuit may include a third electrical feature and a third deformable conductor electrically coupled to the third electrical feature. The fourth flexible circuit may include a fourth electrical feature and a fourth deformable conductor electrically coupled to the third electrical feature. The third and fourth electrical features may be positioned in third and fourth locations of interest on the foot base 2601. For example, when the foot base 2601 is the foot base 1800, the third and fourth locations of interest may be one of front, back, left, and right portions of the sole portion 1802 of the foot base 1800. In some non-limiting aspects, the first, second, third, and fourth locations of interest may be the front, back, left, and right portions of the sole portion 1802 of the foot base. The processor 2632 may receive first, second, third, and fourth signals from the first, second, third, and fourth flexible circuits, respectively; determine first, second, third, and fourth electrical parameters based on the first, second, third, and fourth signals, respectively; correlate the first, second, third, and fourth electrical parameters to first, second, third, and fourth physical parameters associated with the first, second, third, and fourth locations of interest; compare the first, second, third, and fourth physical parameters to each other; and alter the virtual representation of the user based on the comparison of the first, second, third, and fourth physical parameters.

In some non-limiting aspects, one of the electrical features may be the IMU 2608 or replaced with the IMU 2608. In this case, the location of interest (e.g., location of the IMU 2608) can be a center portion of the sole portion 1802. In other examples, the IMU 2608 can be located in any other portions of the foot base 2601. In some non-limiting aspects, the electrical features 2604-1-2604-n may be one of or part of the strain sensor, the inductive coil sensor, and the inflatable circuit discussed above.

In some non-limiting aspects, the user input may be configured to alter physical parameters of the flexible circuit(s). In some non-limiting aspects, the user input may include a pressure applied to the foot base 2601 via a foot of the user and/or a weight transfer of the user over time. The term “weight transfer” may refer to a differential value between a total pressure distribution difference between a default value (e.g., 50/50 left to right and/or front to back) to a second value (e.g., 70/30 left to right and/or front to back) at a given time. The second value may be a dynamic measurement that varies with time. In some non-limiting aspects, the term “weight transfer” may refer to a differential value between a total pressure distribution difference between a first value (e.g., 30/70 left to right and/or front to back) at a first time to a second value (e.g., 70/30 left to right and/or front to back) at a second time later than the first time. Altering the virtual representation of the user may include altering a position or an orientation of the virtual representation of the user relative to the virtual environment. In some non-limiting aspects, altering the virtual representation of the user may also include altering a speed, an acceleration, or a prescribed sequence (e.g., walking, jumping, crouching) of the virtual representation of the user within the virtual environment over time.

The flexible circuits of the foot base 2601 may be able to detect the pressure applied to the flexible circuits of the foot base and weight transfer of the user over time and alter the virtual representation of the user accordingly. By detecting the applied pressure at a given time and the weight transfer over time, the system 2600 (processor 2632) can detect whether the user is leaning toward a particular direction, walking, jumping, etc. In some non-limiting aspects, the leaning direction of the user may indicate the travel direction of the user. For example, if the user is leaning toward a front direction (e.g., a total pressure distribution difference between the electrical feature 1804ₐ in the front portion and the electrical feature 1804_{d} in the back portion of the sole portion 1802 is around 80/20, 90/10, 95/5, or 100/0), this may indicate that the user is moving toward the front direction.

A virtual character in a virtual environment may change a direction of a travel depending on the detected weight transfer of the user over time. For example, if the total pressure distribution is changed from a first value (e.g., 70/30 front to back and 50/50 left to right) at a first time (0.1 seconds) to a second value (e.g., 70/30 front to back and 30/70 left to right) at a second time (0.2 seconds) later than the first time, the system 2600 may change the travel direction of the virtual character from the front direction to the front-right direction.

In some non-limiting aspects, by detecting the frequency of the applied pressure (e.g., number of the times a particular flexible circuit is pressed within a given time), the system 2600 (processor 2632) can detect the speed and acceleration of the user's motion. For example, if the electrical feature 1804ₐ in the front portion of the sole portion 1802 is pressed 10 times for a first period of time (e.g., 0 to 10 seconds), and 20 times for a second period of time (10 to 20 seconds), the system 2600 may detect this acceleration and determine that the motion/travel speed of the user is increased (from 1 step/second to 2 step/second) and alter the virtual representation of the user accordingly. Based on the amount of pressure applied to the electrical features, the system 2600 can also determine the amount of the distance moved by the user in each step. For example, if the amount of the pressure applied to the electrical features 2604 is increased while the user is moving/running in one direction, the system 2600 may determine that the distance moved by the user in each step is also increased.

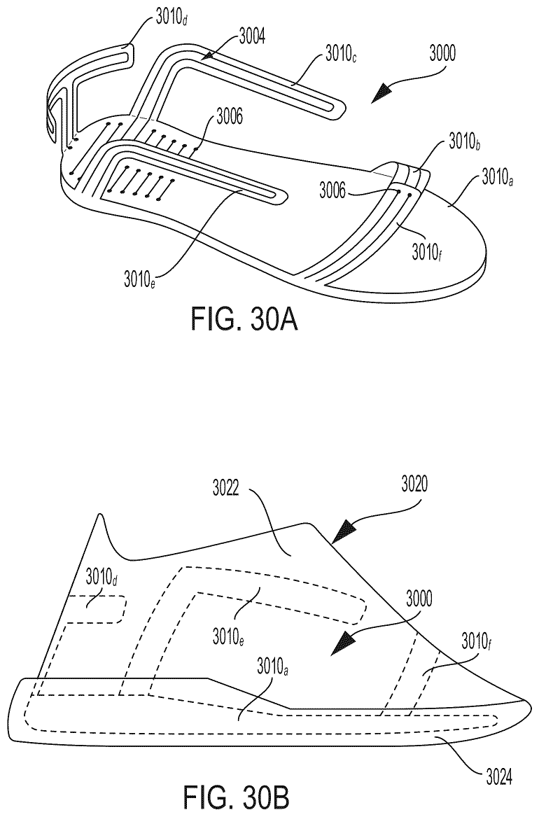

In some non-limiting aspects, the foot base 1800 and/or the upper portion 1850 may be configured/instrumented in a manner similar to the one described in International Patent Application No. PCT/US2022/07882 titled DEVICES, SYSTEMS, AND METHODS FOR MAKING AND USING A PARTIALLY FLUID-FILLABLE CIRCUIT, which was filed on Oct. 27, 2022, the disclosure of which is hereby incorporated by reference in its entirety. For example, referring now to FIGS. 30A and 30B, one implementation of another partially fluid-fillable circuit 3000 is depicted in accordance with at least one non-limiting aspect of the present disclosure. According to the non-limiting aspect of FIGS. 30A and 30B, the partially fluid-fillable circuit 3000 can be constructed similar to any of the partially fluid-fillable circuits disclosed herein. For example, the partially fluid-fillable circuit 3000 can include a plurality of traces 3004 formed from a deformable conductor and various portions of traces 3004 of various portions 3010a-f of the partially fluid-fillable circuit 3000 can be electrically coupled via one or more electrical vias 3006. The partially fluid-fillable circuit 3000 can be constructed from layups configured similar to the layup 1002 of FIG. 10. Likewise, the deformable conductor of the traces 3004 of FIG. 30 and electrical vias 3006 can be configured similar to the deformable conductor of the traces 1004 and vias 1006 of FIG. 10, respectively.

Specifically, the partially fluid-fillable circuit 3000 can include a fluid-fillable portion 3010a and a plurality of non-fluid fillable portions 3010b-f arranged such that the partially fluid-fillable circuit 3000 can be properly installed within a housing 3020. According to the non-limiting aspect, of FIG. 30B, the housing 3020 can be a shoe. For example, the fluid-fillable portion 3010a of the partially fluid-fillable circuit 3000 can form at least a portion of the midsole of a shoe, for example by being over-molded with foam to yield a midsole 3024 of the shoe-shaped housing 3020 and the non-fluid-fillable portions 3010b-f of the partially fluid-fillable circuit 3000 can be laminated and/or otherwise adhered to various portions of an upper portion 3022 of the shoe-shaped housing 3020. Alternatively, the fluid-fillable portion 3010a can form at least a portion of a midsole without being contained within foam, as is known in various commercially available shoe models. In such an alternative and non-limiting example, both the outsole and upper may be directly coupled to the fluid-fillable portion. According to other non-limiting aspects, the number and/or shape of the fluid-fillable portions 3010a and non-fluid fillable portions 3010b-f can be varied in accordance with user preference and/or intended application. For example, the number and/or shape of the fluid-fillable portions 3010a and non-fluid fillable portions 3010b-f can be varied to promote comfort and/or accommodate for space constraints, accordingly.

For example, according to some non-limiting aspects, the non-fluid fillable portions 3010b-f can be used to measure strains in certain portions of the circuit, analyze fit, and/or adjust or suggest adjustments to the tension of a lacing system (e.g., auto-lacing, etc.) For example, some non-limiting aspects can include an auto-lacing application such as those disclosed in U.S. Pat. No. 10,743,620, titled AUTOMATED TENSIONING SYSTEM FOR AN ARTICLE OF FOOTWEAR, and granted Aug. 18, 2020, the disclosure of which is hereby incorporated by reference in its entirety. According to other non-limiting aspects, the lacing system can be passive, or otherwise a traditional lacing system. According to still other non-limiting aspects, the non-fluid fillable portions 3010b-f can host electronic components on certain portions, such as processors, LED arrays or accelerometers, etc., or can be alternately configured to function as buses to a power source or to transmit signals from one portion hosting a sensor for some purpose, as provided above or other functions, to another portion hosting a processor or some other feedback device (e.g., haptic, displays, etc.).

According to some non-limiting aspects, the partially fluid-fillable circuit 3000 can include a LED bank or other electronic components (e.g., a power source, a microprocessor, a logic-based controller, a transceiver, an electrode, a LED bank, a pump etc.) configured to serve any functional and/or decorative purpose. For example, the partially fluid-fillable circuit 3000 can include a microprocessor and/or sensors configured to determine a pressure applied to the fluid-fillable portion 3010a of the partially fluid-fillable circuit 3000. A user, via a pump or other means of inflating the fluid-fillable portion 3010a could subsequently adjust the amount of cushioning provided by the fluid-fillable portion 3010a, based on the determined pressure. According to other non-limiting aspects, the LED bank can provide a visual indication of how much is pressure presently applied to the fluid-fillable portion 3010a.

According to still other non-limiting aspects, a transmitter or transmitter circuit of the partially fluid-fillable circuit 3000 can transmit an indication of the amount of pressure presently applied to the fluid-fillable portion 3010a to a computing device (e.g., a smart phone, a tablet, a laptop computer, a desktop computer, etc.) of the user. According to still other non-limiting aspect, the user can transmit a command to the partially fluid-fillable circuit 3000 via a computing device to either inflate and/or deflate the fluid-fillable portion 3010a of the partially fluid-fillable circuit 3000 thereby, controlling the amount of pressure applied to the fluid-fillable portion 3010a in accordance with the user's preference. For example, according to some non-limiting aspects, the partially fluid-fillable circuit 3000 can be implemented as an electronically controlled bladder system, such as those disclosed in U.S. Pat. No. 9,066,558, titled ELECTRONICALLY CONTROLLED BLADDER ASSEMBLY, and granted Jun. 30, 2015, the disclosure of which is hereby incorporated by reference in its entirety.

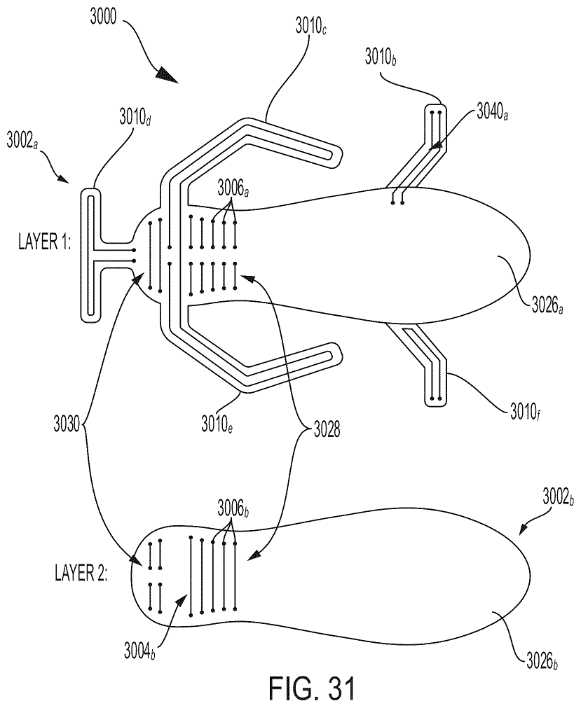

Referring now to FIG. 31, an assembly of the of partially fluid-fillable circuit 3000 of FIG. 30 is depicted in accordance with at least one non-limiting aspect of the present disclosure. According to the non-limiting aspect of FIG. 31, the partially fluid-fillable circuit 3000 can include a first layup 3002a and a second layup 3002b. As depicted in FIG. 31, the first layup 3002a has a base layer 3026a having a geometry that is substantially similar and corresponds with a base layer 3026b of the second layup 3002b. For example, both the base layers 3026a, 3026b of the first layup 3002a and the second layup 3002b appear to have an insole configuration or “foot-shape” when viewed from above. However, as depicted in FIG. 31, portions of the first layup 3002a that will form the non-fluid-fillable portions 3010b-f of the partially fluid-fillable circuit 3000 extend away from the insole-shaped base portion 3026a of the first layup 3002a. Accordingly, the base layer 3026a of the first layup 3002a can be positioned over the base layer 3026b of the second layup 3002b, and the first and second layups 3002a, 3002b can be unitized about a flanged seal (not shown) configured to traverse a perimeter of the base portions 3026a, 3026b, thereby defining a cavity between inner surfaces of the base layers 3026a, 3026b. In other words, upon unitization, the base layer 3026a of the first layup 3002a and the base layer 3026b of the second layup 3002b can form the fluid-fillable portion 3010a of the partially fluid-fillable circuit 3000. The fluid-fillable portion 3010a can be selectively inflated and/or deflated using any of the techniques previously disclosed.

According to the non-limiting aspect of FIG. 31, both the first and second layups 3002a, 3002b can include a number of electrical structures, such as traces 3004a, 3004b formed from deformable conductors. As depicted in FIG. 31, the traces 3004a, 3004b can traverse portions of the base layers 3026a, 3026b and the plurality of non-fluid-fillable portions 3010b-f of the first and second layups 3002a, 3002b. For example, portions of the traces 3004a, 3004b can be configured as a strain sensor, a pressure sensor, an antenna circuit, and/or a proximity sensor of the partially fluid-fillable circuit 3000. According to some non-limiting aspects, traces 3004a, 3004b on the first and second layups 3002a, 3002b can be electrically coupled to various electronic components (e.g., a power source, a microprocessor, a logic-based controller, a transceiver, an electrode, a LED bank, a pump etc.).

Moreover, the traces 3004a on the first layup 3002a of FIG. 31 can be electrically coupled to traces 3004b on the second layup 3002b via a plurality of electrical vias 3006a, 3006b on the first and second layup 3002a, 3002b, thereby establishing an electrical communication between the first layup 3002a and the second layup 3002b. The electrical vias 3006a, 3006b can be configured to establish different channels of electrical communication between various layers of the layups 3002a, 3002b and aggregated and/or arranged on the first and second layup 3002a, 3002b accordingly. For example, according to the non-limiting aspect of FIG. 31, the electrical vias 3006a, 3006b are arranged according to whether or not they establish electrical communication between a first layer 3028 of the layups 3002a, 3002b and a second layer 3030 of the layups 3002a, 3002b, thereby creating different channels of electrical communication throughout the partially fluid-fillable circuit 3000.

In other words, the electrical vias 3006a, 3006b of FIG. 31 can be configured to serve as contacts for electrically coupling various traces 3004a, 3004b of the first and second layups 3002a, 3002b. According to the non-limiting aspect of FIG. 31, this can include electrically coupling traces 3004a positioned on the plurality of non-fluid-fillable portions 3010b-f to traces 3004b positioned on the second layup 3002b. However, according to other non-limiting aspects, the traces 3004a positioned on the plurality of non-fluid-fillable portions 3010b-f can extend to the traces 3004a positioned on the base portion 3026a of the first layup 3026, which can then be to electrically coupled to traces 3004b positioned on the second layup 3002b via electrical vias 3006a positioned on a heel portion of the base portion 3026a of the first layup 3026.

According to some non-limiting aspects, traces 3004b of the second layup 3002b of FIG. 31 can be configured as a bus circuit for transporting electrical power to the traces 3004a and/or any electronic components on the first layup 3002a. Accordingly, the second layup 3002b can be configured to transport electrical power and current throughout the partially fluid-fillable circuit 3000 via the traces 3004b on the second layup 3002b, through the electrical vias 3006a, 3006b, and through the traces 3004a on the first layup 3002a. Accordingly, the partially fluid-fillable circuit 3000 of FIG. 31 can perform any of the functions described in reference to any of the aforementioned circuits as the fluid-fillable portion 3010a is selectively inflated and deflated.

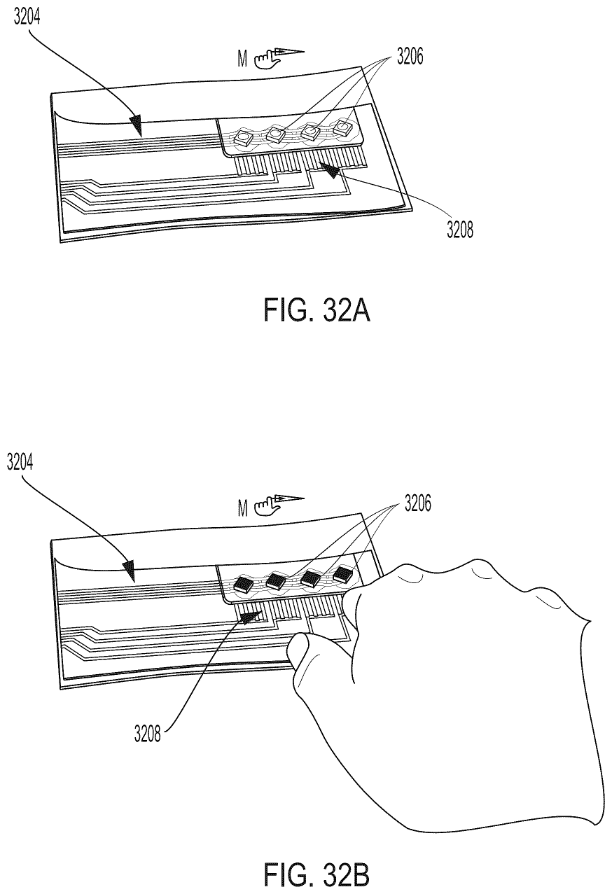

According to some non-limiting aspects, the one or more of the traces described in the present disclosure can be implemented as part of a capacitive touch interface. For example, referring now to FIGS. 32A and 32B, a capacitive touch interface 3200 configured for use with any of the aforementioned partially-fluid-fillable circuits is depicted in accordance with at least one non-limiting aspect of the present disclosure. According to the non-limiting aspect of FIGS. 32A and 32B, the capacitive touch interface 3200 can include a flexible circuit formed from traces 3204 deformable conductors. The traces 3204 can be coupled to an array of LEDs 3206, which can also be electrically coupled to another arrangement of deformable traces 3208 configured to function a capacitive sensor, for example, comprising coils. The traces 3208 can be embedded within a layup and/or a fabric from which the circuit or interface 3200 is constructed. As such, if a user were to interact with one or more of the traces 3208 configured to function as a capacitive sensor, one or more LEDs 3206 of the array can be illuminated, as depicted in FIGS. 32A and 32B. In FIG. 32A, the user performs motion M by sliding their finger across the traces 3708 configured to function as a capacitive sensor and thus, all of the LEDs 3206 of the array have illuminated, as depicted in FIG. 32B. Alternately and/or additionally, the use can press their finger lightly to activate one or more LEDs 3206, selectively.

Still referring to FIGS. 32A and 32B, it shall be appreciated that a capacitive touch interface 3200 can be implemented to receive a user input. For example, by pressing the traces 3208 configured to function a capacitive sensor, the user can provide signals to and from an electronic component communicably coupled to the capacitive touch interface 3200. For example, according to some non-limiting aspects, the electronic component can include a microprocessor configured to selectively inflate and/or deflate a fluid-fillable portion of the partially-fluid-fillable circuit in response to user inputs provided via the various traces 3208 of the capacitive touch interface 3200. Additionally, the array of LEDs 3206 can provide a visual indicia as to the degree of inflation of a fluid-fillable portion of the partially-fluid-fillable circuit, as various LEDs 3206 can be illuminated in accordance with various user inputs. Of course, according to other non-limiting aspects, pressing the traces 3208 of the capacitive touch interface 3200 can be configured to transmit any other command to any other electronic component communicably coupled to the partially-fluid-fillable circuit.

Various aspects of the subject matter described herein are set out in the following numbered clauses:

Clause 1: A system configured to control a virtual representation of a user within a virtual environment, the system including: a foot base configured to receive a user input, wherein the foot base includes a flexible circuit including an encapsulated deformable conductor; a processor communicably coupled to the flexible circuit; and a memory configured to store instructions that, when executed by the processor, cause the processor to: receive a signal from the flexible circuit; determine an electrical parameter based on the received signal; correlate the determined electrical parameter to a physical parameter of the flexible circuit; and alter the virtual representation of the user based on the correlation.

Clause 2: The system of clause 1, wherein the user input is configured to alter physical parameters of the flexible circuit.

Clause 3: The system of clauses 1-2, wherein the user input includes at least one of a pressure applied to the foot base via a foot of the user and a weight transfer of the user over time.

Clause 4: The system of clauses 1-3, wherein altering the virtual representation of the user includes altering a position or an orientation of the virtual representation of the user relative to the virtual environment.

Clause 5: The system of clauses 1-4, wherein altering the virtual representation of the user includes altering a speed, an acceleration, or a prescribed sequence of the virtual representation of the user within the virtual environment over time.

Clause 6: The system of clauses 1-5, wherein the encapsulated deformable conductor includes a pattern of traces.

Clause 7: The system of clause 6, wherein the pattern of traces is configured to serve as a strain sensor.

Clause 8: The system of clause 6, wherein the pattern of traces is configured to serve as an inductive coil sensor.

Clause 9: The system of clause 6, wherein the encapsulated deformable conductor and/or the pattern of traces is configured to change its viscosity in response to a strain applied, by the user input, to the flexible circuit.

Clause 10: The system of clauses 1-9, wherein the flexible circuit is operatively coupled to a cavity that is fillable with a fluid.

Clause 11: The system of clauses 1-10, wherein the flexible circuit includes a plurality of flexible circuits having a first flexible circuit and a second flexible circuit, wherein the first flexible circuit includes: a first encapsulated deformable conductor of the encapsulated deformable conductor; and a first electrical feature electrically coupled to the first encapsulated deformable conductor, wherein the first electrical feature is positioned in a first location of interest on the foot base, and wherein the second flexible circuit includes: a second encapsulated deformable conductor of the encapsulated deformable conductor; and a second electrical feature electrically coupled to the second encapsulated deformable conductor, wherein the second electrical feature is positioned in a second location of interest on the foot base.

Clause 12: The system of clause 11, wherein the instructions, when executed by the processor, further cause the processor to: receive first and second signals from the first and second flexible circuits, respectively; determine first and second electrical parameters based on the first and second signals, respectively; correlate the first and second parameters to first and second physical parameters, respectively, wherein the first and second physical parameters are associated with the first and second locations of interest, respectively; compare the first and second physical parameters with each other; and alter the virtual representation of the user based on the comparison of the first and second physical parameters associated with the first and second locations of interest.

Clause 13: The system of clause 12, wherein the foot base includes a sole portion, and the first and second locations of interest are one of front, back, left, and right portions of the sole portion of the foot base, wherein the front, back, left, and right portions of the sole portion are configured to be associated with front, back, left, and right portions of a sole of the user, respectively.

Clause 14: The system of clause 11, wherein the foot base further includes a third flexible circuit including: a third encapsulated deformable conductor of the encapsulated deformable conductor; and a third electrical feature electrically coupled to the third encapsulated deformable conductor, wherein the third electrical feature is positioned in a third location of interest on the foot base, and wherein the instructions, when executed by the processor, further cause the processor to: receive a third signal from the third flexible circuit; determine a third electrical parameter based on the third signal; correlate the third electrical parameter to a third physical parameter associated with the third location of interest; compare the third physical parameter associated with the third location of interest to the first and second physical parameters; and wherein the alteration of the virtual representation of the user is further based on the comparison of the third physical parameter to the first and second physical parameters.

Clause 15: The system of clause 14, wherein the third electrical feature includes an inertial measurement unit (“IMU”).

Clause 16: The system of clauses 14-15, wherein the third location of interest is a center portion of a sole portion of the foot base.

Clause 17: The system of clause 14, wherein the foot base includes an upper portion configured to surround an instep and/or an ankle of the user.

Clause 18: The system of clause 17, wherein the third location of interest is an instep portion of the upper portion of the foot base, wherein the instep portion is configured to be associated with the instep of the user.

Clause 19: The system of clause 17, wherein the third location of interest is an ankle portion of the upper portion of the foot base, wherein the ankle portion is configured to be associated with the ankle of the user, wherein the third flexible circuit is configured to detect dorsiflexion and plantar flexion of a foot of the user.

Clause 20: The system of clauses 14-19, wherein the first, second, and third electrical features include at least one of a strain sensor, an inductive coil sensor, and an inflatable circuit.

Clause 21: The system of clauses 1-20, wherein the foot base includes at least one of a midsole, an insole, and an outsole of a shoe.

Clause 22: The system of clauses 1-21, wherein the foot base includes a sock, a mat, or a sheet.

Clause 23: The system of clauses 1-22, wherein the encapsulated deformable conductor comprises: a substrate layer; an encapsulation layer; and a deformable conductor between the substrate layer and the encapsulation layer, the deformable conductor configured to generate varying signals in response to the received user input.

Clause 24: The system of clauses 1-23, wherein the foot base further comprises a foot base substrate and the encapsulated deformable conductor is disposed in or on the foot base substrate, wherein the foot base substrate is configured to deform in response to a strain applied, by the user input, to the substrate.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The invention is claimed as follows:

1. A system configured to control a virtual representation of a user within a virtual environment, the system comprising:
a foot base configured to receive a user input, wherein the foot base comprises a flexible circuit comprising an encapsulated deformable conductor;
a processor communicably coupled to the flexible circuit; and
a memory configured to store instructions that, when executed by the processor, cause the processor to:
receive a signal from the flexible circuit;
determine an electrical parameter based on the received signal;
correlate the determined electrical parameter to a physical parameter of the flexible circuit; and
alter the virtual representation of the user based on the correlation.

2. The system of claim 1, wherein the user input is configured to alter physical parameters of the flexible circuit.

3. The system of claim 2, wherein the user input comprises at least one of a pressure applied to the foot base via a foot of the user and a weight transfer of the user over time.

4. The system of claim 1, wherein altering the virtual representation of the user comprises altering a position or an orientation of the virtual representation of the user relative to the virtual environment.

5. The system of claim 1, wherein altering the virtual representation of the user comprises altering a speed, an acceleration, or a prescribed sequence of the virtual representation of the user within the virtual environment over time.

6. The system of claim 1, wherein the encapsulated deformable conductor comprises a pattern of traces.

7. The system of claim 6, wherein the pattern of traces is configured to serve as a strain sensor.

8. The system of claim 6, wherein the pattern of traces is configured to serve as an inductive coil sensor.

9. The system of claim 6, wherein the encapsulated deformable conductor and/or the pattern of traces is configured to change its viscosity in response to a strain applied, by the user input, to the flexible circuit.

10. The system of claim 1, wherein the flexible circuit is operatively coupled to a cavity that is fillable with a fluid.

11. The system of claim 1, wherein the flexible circuit comprises a plurality of flexible circuits having a first flexible circuit and a second flexible circuit,
wherein the first flexible circuit comprises:
a first encapsulated deformable conductor of the encapsulated deformable conductor; and
a first electrical feature electrically coupled to the first encapsulated deformable conductor, wherein the first electrical feature is positioned in a first location of interest on the foot base, and
wherein the second flexible circuit comprises:
a second encapsulated deformable conductor of the encapsulated deformable conductor; and
a second electrical feature electrically coupled to the second encapsulated deformable conductor, wherein the second electrical feature is positioned in a second location of interest on the foot base.

12. The system of claim 11, wherein the instructions, when executed by the processor, further cause the processor to:
receive first and second signals from the first and second flexible circuits, respectively;
determine first and second electrical parameters based on the first and second signals, respectively;
correlate the first and second parameters to first and second physical parameters, respectively, wherein the first and second physical parameters are associated with the first and second locations of interest, respectively;
compare the first and second physical parameters with each other; and
alter the virtual representation of the user based on the comparison of the first and second physical parameters associated with the first and second locations of interest.

13. The system of claim 12, wherein the foot base comprises a sole portion, and the first and second locations of interest are one of front, back, left, and right portions of the sole portion of the foot base, wherein the front, back, left, and right portions of the sole portion are configured to be associated with front, back, left, and right portions of a sole of the user, respectively.

14. The system of claim 11, wherein the foot base further comprises a third flexible circuit comprising:
a third encapsulated deformable conductor of the encapsulated deformable conductor; and
a third electrical feature electrically coupled to the third encapsulated deformable conductor, wherein the third electrical feature is positioned in a third location of interest on the foot base, and
wherein the instructions, when executed by the processor, further cause the processor to:
receive a third signal from the third flexible circuit;
determine a third electrical parameter based on the third signal;
correlate the third electrical parameter to a third physical parameter associated with the third location of interest;
compare the third physical parameter associated with the third location of interest to the first and second physical parameters; and
wherein the alteration of the virtual representation of the user is further based on the comparison of the third physical parameter to the first and second physical parameters.

15. The system of claim 14, wherein the third electrical feature comprises an inertial measurement unit (“IMU”).

16. The system of claim 14, wherein the first, second, and third electrical features comprise at least one of a strain sensor, an inductive coil sensor, and an inflatable circuit.

17. The system of claim 1, wherein the foot base comprises at least one of a midsole, an insole, and an outsole of a shoe.

18. The system of claim 1, wherein the foot base comprises a sock, a mat, or a sheet.

19. The system of claim 1, wherein the encapsulated deformable conductor comprises:
a substrate layer;
an encapsulation layer; and
a deformable conductor between the substrate layer and the encapsulation layer, the deformable conductor configured to generate varying signals in response to the received user input.

20. The system of claim 1, wherein the foot base further comprises a foot base substrate and the encapsulated deformable conductor is disposed in or on the foot base substrate, wherein the foot base substrate is configured to deform in response to a strain applied, by the user input, to the substrate.
